SEQUENCE LISTING (43 sequences)

SEQ ID NO: 1 - DNA, 14 bp, Artificial Sequence
  Synthetic construct
tttttttttttttt

SEQ ID NO: 2 - DNA, 8 bp, Artificial Sequence
  Synthetic construct
ggaattcc

SEQ ID NO: 3 - DNA, 18 bp, Artificial Sequence
  Synthetic construct
ttttttttggtttttttt

SEQ ID NO: 4 - DNA, 20 bp, Artificial Sequence
  Synthetic construct
ggcattttaagttgctgtcg

SEQ ID NO: 5 - RNA, 20 nt, Artificial Sequence
  Synthetic construct
ggcauuuuaaguugcugucg

SEQ ID NO: 6 - RNA, 28 nt, Artificial Sequence
  Synthetic construct
cgacagcaacuuaaaaugccugggaaga

SEQ ID NO: 7 - DNA, 33 bp, Artificial Sequence
  Synthetic construct
cacaggagaaggaggaggtggaggaggagggct

SEQ ID NO: 8 - DNA, 33 bp, Artificial Sequence
  Synthetic construct
agccctcctcctccacctcctccttctcctgtg

SEQ ID NO: 9 - DNA, 28 bp, Artificial Sequence
  Synthetic construct
gggtggtggtggtggtggtggttgtggt

SEQ ID NO: 10 - DNA, 16 bp, Artificial Sequence
  Synthetic construct
ttcctctttggggtgt

SEQ ID NO: 11 - RNA, 26 nt, Artificial Sequence
  Synthetic construct
ccaugaacuuucugcugucuugggug

SEQ ID NO: 12 - DNA, 37 bp, Artificial Sequence
  Synthetic construct
nnagtgcacannngaaagacgacannntgtgcactnn

SEQ ID NO: 13 - RNA, 117 nt, Artificial Sequence
  Synthetic construct
guccuguccguaugacagagaagucaaccagagaaacacacguugugguauauuaccuggucaagaaguucccuguuccuuuuuuucccgagccuugucccuugaagagaacugacc

SEQ ID NO: 14 - RNA, 22 nt, Artificial Sequence
  Synthetic construct
guucucuucaagggacaaggcu

SEQ ID NO: 15 - RNA, 67 nt, Artificial Sequence
  Synthetic construct
ggucaagaaguucccuguuccuuuuuuucccgagccuugucccuugaagagaacagaccguccuguc

SEQ ID NO: 16 - RNA, 68 nt, Artificial Sequence
  Synthetic construct
acugguccauuauaugguguugcacacaaacagaccaacugaagagacaguaugccuguccugccagu

SEQ ID NO: 17 - DNA, 36 bp, Artificial Sequence
  Synthetic construct
acccggggatcctctagagttcgacctgcaggggcc

SEQ ID NO: 18 - DNA, 52 bp, Artificial Sequence
  Synthetic construct
cctaatacgactcactataggcaacgttctcttcaagggacaaggctgcatg

SEQ ID NO: 19 - DNA, 54 bp, Artificial Sequence
  Synthetic construct
catgcagccttgtttcccttgaagagaacgttgcctatagtgagtcgtattagg

SEQ ID NO: 20 - RNA, 25 nt, Artificial Sequence
  Synthetic construct
aggagggagaacagaaacuccagaa

SEQ ID NO: 21 - RNA, 121 nt, Artificial Sequence
  Synthetic construct
guccugugcguaugacagagaagucaaccagagaaacacacguugugguauauuaccuggucauucuggaguuucuguucucccuccucuuuguccucccucuugucuuuccuccuugacc

SEQ ID NO: 22 - RNA, 20 nt, Artificial Sequence
  Synthetic construct
gugccuaugucucagccucu

SEQ ID NO: 23 - RNA, 108 nt, Artificial Sequence
  Synthetic construct
guccugugcguaugacagagaagcugaaccagagaaacacacguugugguauauuaccuggucagaggcugagacauaggcacaacaacaacaacaacaacaucagcc

SEQ ID NO: 24 - RNA, 125 nt, Artificial Sequence
  Synthetic construct
guccugugcguaugacagagaagucaaccagagaaacacacguugugguauauuaccuggucauucuggaguuucuguucucccuccucuuuguccucccucuugucuuuccuccuccucugacc

SEQ ID NO: 25 - RNA, 28 nt, Artificial Sequence
  Synthetic construct
ucacgacagcaacuuaaaaugccuggga

SEQ ID NO: 26 - RNA, 113 nt, Artificial Sequence
  Synthetic construct
guccuguccguaugacagagaagucaaccagagaaacacacguugugguauauuaccuggucaccuccuccuccuccuccuccuccggcauuuuaaguugcugucgccugacc

SEQ ID NO: 27 - DNA, 69 bp, Artificial Sequence
  Synthetic construct
ttctcccgctcctcctcgggggtcgccgtgttccggcggtcgaggtaccagagtgacgccgaggccggg

SEQ ID NO: 28 - DNA, 30 bp, Artificial Sequence
  Synthetic construct
cgtgacanggctccngctcactntgtcacg

SEQ ID NO: 29 - DNA, 29 bp, Artificial Sequence
  Synthetic construct
cgtgacanacaaggnccagcgntgtcacg

SEQ ID NO: 30 - DNA, 9 bp, Artificial Sequence
  Synthetic construct
nacacgtga

SEQ ID NO: 31 - DNA, 8 bp, Artificial Sequence
  Synthetic construct
ngtgcact

SEQ ID NO: 32 - DNA, 40 bp, Artificial Sequence
  Synthetic construct
nnngtgcacnnnnnnntccngctcnnnnnnngtgcacnnn

SEQ ID NO: 33 - DNA, 41 bp, Artificial Sequence
  Synthetic construct
nnnagtgcacnnnnnnnaggnccannnnnnngtgcactnnn

SEQ ID NO: 34 - RNA, 62 nt, Artificial Sequence
  Synthetic construct
uacauagagacaauggcagcaauuucaccaguacuacaguuaaggccgccuguuggugggcg

SEQ ID NO: 35 - RNA, 271 nt, Artificial Sequence
  Synthetic construct
ggggacccccccggaagggggggacgaggugcgggcaccucguacgggaguucgaccgugacgcucuagcaggcggccuuauuugaccguccugucuuuugcugccauugcuagagcacgggcuagcgcuuucgcgcucucccaggugacgccucgugaagaggcgcgaccuucgugcguuucggugacgcacgagaaccgccacgcugcuucgcagcguggcuccuucgcgcagcccgcugcgcgaggugacccccgaagggggguuccc

SEQ ID NO: 36 - RNA, 45 nt, Artificial Sequence
  Synthetic construct
ggacagguaagagaucaggcugaacaucuuaagacagcaguacaa

SEQ ID NO: 37 - RNA, 60 nt, Artificial Sequence
  Synthetic construct
cuguccugccaguuuuuguacuggugucuuaagauguucagccugaucucuuaccugucc

SEQ ID NO: 38 - RNA, 24 nt, Artificial Sequence
  Synthetic construct
gucaaggaguaauagaaucuauga

SEQ ID NO: 39 - RNA, 30 nt, Artificial Sequence
  Synthetic construct
ucauagauucuannnnguauauuaccuggu

SEQ ID NO: 40 - RNA, 41 nt, Artificial Sequence
  Synthetic construct
gacagagaagucaacaagagaaacannnnuuacuccuugac

SEQ ID NO: 41 - RNA, 101 nt, Artificial Sequence
  Synthetic construct
ucauagauucuannnnguauauuaccugguuuuuuuuuuuuuugaccguccuguccguaugacagagaagucaacaagagaaacannnnuuacuccuugac

SEQ ID NO: 42 - RNA, 35 nt, Artificial Sequence
  Synthetic construct
nunnnnnnnnaaagnnnnagnagucnnnnnnnnnn

SEQ ID NO: 43 - RNA, 37 nt, Artificial Sequence
  Synthetic construct
nunnaaagnnnnnnnnnnnnagnagucnnnnnnnnnn

(19) United States
(12) Patent Application Publication
(10) Pub. No.: US 2006/0074041 A1
(43) Pub. Date: Apr. 6, 2006

(54) Antisense and antigene therapeutics with improved binding properties and methods for their use

(75) Inventors: Brian H. Johnston, Scotts Valley, CA (US); Sergei A. Kazakov, Los Gatos, CA (US); Kevin O. Kisich, Lafayette, CO (US)

Correspondence Address: MORRISON & FOERSTER LLP, 755 PAGE MILL RD, PALO ALTO, CA, 94304-1018, US

(73) Assignee: Somagenics, Inc., Santa Cruz, CA (US)

(21) Appl. No.: 11/228,705

(22) Filed: Sep. 16, 2005

Related U.S. Application Data
(63) Continuation of application No. 09/137,059, filed on Aug. 20, 1998, now abandoned.
(60) Provisional application No. 60/056,742, filed on Aug. 20, 1997.

(51) Int. Cl. A61K 48/00; C07H 21/02

(52) U.S. Cl. 514/44; 536/23.1

(57) ABSTRACT

The present invention is directed to novel nucleic acid molecules and methods for their use. More specifically, the novel nucleic acid molecules of the present invention are capable of tightly and specifically interacting with a target molecule of interest not only through standard Watson-Crick base pairing, but also through additional features which allow the antisense molecules to become topologically “locked” onto the target nucleic acid, thereby imparting improved transcription and translation inhibitory properties.

### FIELD OF THE INVENTION

The present invention relates generally to antisense and antigene oligonucleotides and their use as probes as well as diagnostic and therapeutic agents, and more particularly to antisense and antigene oligonucleotides which are capable of topologically linking to target nucleic acid molecules so as to impart tight binding characteristics and, in turn, improved translation and transcription inhibitory properties. The present invention also relates to novel methods for the platination of oligonucleotides to improve their antisense and triplex-forming properties and to allow those oligonucleotides to bind to double-stranded DNA through an antisense mechanism.

### BACKGROUND OF THE INVENTION

Antisense approaches to the therapeutic modulation of gene expression have been shown to be effective both in cultured cells and in whole animals (Crooke, Antisense Nucleic Acid Drug Dev. 8:115-122 (1998), Matteucci and Wagner, Nature 384:20-21 (1996) and Mesmaeker et al., Acc. Chem. Res. 28:366-374 (1995)). However, the standard antisense method, involving delivery of oligonucleotides modified for nuclease resistance, has several difficulties: Toxicity of phosphorothioate and other derivaties is a concern, cell-specific delivery is difficult, and levels with target cells can only be controlled by frequency of injection. In addition, automated synthetic methods for production of antisense oligonucleotides remain expensive, and may prevent the use of clinically successful antisense therapeutics for some indications. A stubborn problem with the introduction of exogenous oligonucleotides is that in many cells they are taken up by endocytosis and confined to endosomes rather than reaching the nucleus. This compartmentalization problem can be circumvented by transcription of antisense genes in situ to generate antisense RNAs or, often, by the delivery of oligonucleotides using cationic lipids. The in situ approach has the added advantages of avoiding unnatural, toxic DNA derivatives and permitting continuous exposure to an antisense molecule or a ribozyme if the introduced DNA is stably expressed. Moreover, with appropriate regulated promoters, delivery can be regulated by external signals. However, antisense RNA cannot take advantage of RNase H cleavage to achieve effective targeting of coding regions. Modifications at the ribose 2′ position which improve effectiveness also prevent RNase H activity. Without activity by RNase H, sense-antisense complexes within coding regions are disrupted by the passage of ribosomes during translation. Double-stranded RNA modification enzymes can also disrupt sense-antisense complexes. Thus, at present it is not possible to achieve blockage of translation in coding regions by hybridization with antisense RNAs. Most, if not all, antisense leads being developed for (or presently in) clinical trials bind mRNA in noncoding regions.

In light of the above, there is a significant need for novel antisense and antigene compositions and methods for using those compositions which effectively provide for the down regulation of protein expression both in vitro and in vivo without many of the limitations inherent in the standard antisense approaches discussed above. Specifically, there is a need for novel antisense and antigene therapeutics which are capable of resisting dissociation after binding to a target RNA molecule through various forms of topological linkage, thereby effectively inhibiting transcription and/or translation from the target.

### SUMMARY OF THE INVENTION

The present invention is directed to improved antisense and antigene oligonucleotide compositions and methods for down-regulating gene expression in cells using novel antisense and antigene oligonucleotides that are capable of topologically linking to target nucleic acid molecules, thereby imparting tight binding properties and the ability to resist dissociation from the target. More particularly, the present invention is directed to a new generation of antisense and antigene agents for the specific control of gene expression. These agents bind to RNA and/or DNA target molecules not merely by the strength of Watson-Crick pairing (as do standard antisense agents), but employ additional features that “lock” the antisense or antigene molecule onto the target nucleic acid, thereby making it highly resistant to dissociation promoted by helicases, ribosomes or modifying enzymes. We herein demonstrate success in achieving extremely tight binding and have identified mechanisms responsible for this tight binding. Moreover, we herein establish that this method is very effective in blocking ribosome scanning in cell-free translation systems as well as in intact cells. This approach of using nucleic acid structural considerations to topologically “padlock” the antisense or antigene and target molecules together is a significant advance, for antisense and antigene therapy in general, for gene function analysis and target validation and for gene therapy, as a means for the controlled and cell-specific delivery of antisense and antigene molecules, in particular. Methods employing these antisense oligonucleotides both in vitro and in vivo as well as kits comprising them are also provided. Methods for the platination of oligonucleotides, for example, to improve their antisense and triplex-forming properties are also provided. In addition, we provide methods for detecting and amplification of nucleic acids based on mechanistic features of some of these antisense constructs.

### BRIEF DESCRIPTION OF THE DRAWINGS

FIGS. 1A-H. Exemplary Schemes for Employing Antisense Oligonucleotides Which Become Topologically Linked to the Target Molecule. A. Presented is a general scheme showing a nucleic acid molecule topologically linked to a single-stranded target. The ball represents any of various ways for the ends of this molecule to interact following hybridization with the target with creation of at least one turn of helical interwinding. The third-strand interaction to form a triplex is optional. In one embodiment, the ball comprises a hairpin ribozyme moiety (see FIG. 2). B. Presented is a simple version of a padlock RNA, where linkage is achieved by hybridization of complementary sequences on the ends. Triplex formation is optional. C-E. Presented are variants of A in which the antisense sequence is split. This version is particularly suited to use of a separate clasp molecule (ball) to afford cell-specific target binding. Sense-antisense hybridization is unstable in the absence of the clasp molecule. FIGS. 1C and 1D show cases in which the target is a single-stranded RNA, with and without triplex formation, respectively. E shows a case in which target is a double-stranded nucleic acid molecule, with target binding through triplex formation. F. Presented is a variant of D in which closure is by simple base-pairing of the ends after topological linkage, showing helical interwinding. G and H show a variant of FIG. 1C in which linkage is achieved by a single helical turn, eliminating the need for any unpaired regions. G shows binding of a padlock DNA to the beginning of the coding region of human VEGF mRNA. The ends of the padlock DNA create a binding site for c-myc. Residues labeled n are to be determined by optimization experiments such that strong binding is dependent on the presence of a physiologically relevant concentration of c-myc (or other member of the myc family). H. Presented is a space-filling model of FIG. 1G, created by docking a B-form DNA duplex with one turn of an A-form RNA-DNA duplex, linking the backbone chains, and performing energy minimization.

FIGS. 2A-F. Schematic Representation of the Structures of Various ATR 1 Antisense RNA Species and Hairpin Ribozyme Structure. A. Presented is the sequence of the primary transcript pre-ATR 1 (R2) showing its functional domains including the proximal (P5 and P8) and distal (D8 and D9) substrate portions of the hairpin ribozyme (HPR) relative to the catalytic core thereof (E48) (where the numbers refer to the length of the segment in nucleotides), and the antisense (A) and triplex (T) forming regions. Also presented are the fragments generated as products of either R2 self-processing (R3a, R3b and R4) or transcription using a shorter template lacking the catalytic hairpin ribozyme domain (AT). The terminal groups are as shown: “ppp”, 5′-triphosphate; “OH”, 5′ or 3′ hydroxyl; and “>p”, 2′,3′-cyclic phosphate. B. Presented is the putative secondary structure of the complex formed between the murine TNFα mRNA target and the “folded” form of the ATR 1 antisense RNA (R1 or R4). C. Presented is the putative secondary structure of the complex formed between the TNFα mRNA target and the AT antisense RNA, containing only the antisense and triplex forming regions but lacking the hairpin ribozyme. D. Presented is the secondary structure and sequence of the essential core of HPR, showing domains referred to in the text. E. Autoradiograph of a 6% denaturing polyacrylamide gel showing RNA species produced by self-processing of primary HPR1 transcript. Lane 1, 5S RNA marker, lane 2, internally ³²P-labeled RNAs showing all species, lane 3, RNAs labeled at their 3′ termini by ligation to 5′-[³²P]ₚCp using T4 ligase. R2 and R3 are unprocessed and partially processed primary transcripts, whereas R1 is circular HPR and R4 is linear HPR. F. 6% denaturing polyacrylamide gel showing RNA species produced by self-processing of primary HPR1 transcript. Lane 1, gel purified circle, lane 2, gel purified linear, lanes 3 and 4, circle (R1) and linear HPR(R4), showing self-cleavage and self-ligation, respectively, after incubating with Mg²⁺-containing buffer for 1 hour at 37° C.

FIG. 3. Superiority of ATR 1 (Antisense-Triplex-Ribozyme) RNA over AT (Antisense-Triplex) RNA in Stability of the Complexes Formed with TNFα Target RNA. FIG. 3 shows an autoradiogram made after polyacrylamide gel electrophoresis of the gel purified linear ATR 1 RNA (“R4” form) in equilibrium with its circular form (“R1” form) (lanes 1-6) and AT RNA (lanes 7-12), after incubation alone (lanes 1, 4, 7 and 10) and either with 0.1 μg/μl (lanes 2, 5, 8 and 11) or 0.2 μg/μl (lanes 3, 6, 9, and 12) TNF1 RNA. All samples were mixed with equal volumes of 2×FLS (standard gel loading solution containing 90% formamide and 10 mM EDTA) and incubated either for 5 min. at 37° C. (lanes 1-6) or for 2 min. at 95° C. (lanes 7-12) before electrophoresis. “S” corresponds to the start location of the gel and “SC” refers to the location of the strongly interacting RNA complexes.

FIGS. 4A-B. TNFα-Luciferase Fusion Targets and Inhibition of Expression by ATR Antisense RNAs. A. Presented is a schematic diagram and the nucleotide sequence of a DNA template for the TNFα-luciferase fusion gene. B. Presented is the inhibition of luciferase expression detected by a luminescence assay as a result of the pre-hybridization of the luciferase-coding mRNA (PS or PTS) with either AT, ATR 1, or a control RNA lacking the antisense sequence folowed by translation using a rabbit reticulocyte system.

FIG. 5. Introduction of Hairpin Ribozyme Derivative/Cationic Lipid Complex to Macrophages In Vivo. FIG. 5 shows the number of molecules taken up by macrophages following intraperitoneal administration of a hairpin ribozyme derivative m101 in association with various cationic lipid delivery vehicles.

FIGS. 6A-D. Phase Contrast and Fluorescence Microscopy of Hairpin Ribozyme Minimonomer Constructs in Murine Macrophages Following Intraperitoneal Administration. A. Presented is the phase contrast image of responsive murine peritoneal macrophages 24 hours after administration of 10 μg of fluorescein-12-UTP m101 complexed at a 3:1 charge ratio with Lipofectamine in 1 ml of Hanks Balanced Salt Solution (HBSS). B. Present is the fluorescent image of A, showing fluorescein signal in the majority of macrophages, but not lymphocytes. C. Presented is the phase contrast image of responsive murine peritoneal macrophages 24 hours after administration of 10 μg of fluorescein-12-UTP m101 without Lipofectamine. D. Presented is the fluorescent image of C.

FIG. 7. ATR Antisense RNAs Targeted Against Regions of TNFα or VCAM and Expected Secondary Structures of Their Complexes. FIG. 7 presents the ATR antisense RNAs directed against regions of TNFα (ATR 16a, ATR 16b and ALR 229) or VCAM (VALR 1) and the expected secondary structures of their complexes with their specific targets.

FIG. 8. Padlock Complexes That Can Block HER-2 mRNA Around the Translation Start (HER-5′). Oligonucleotide HERMYC1 targets a sequence before the start site (bold); HERMYC2 targets a sequence within the coding region. The vertical sequences contain the c-myc/max heterodimer consensus binding site. “xxx” represents either (CUU)ₙ or ethylene glycol residues for linker regions.

FIG. 9. Scheme For Using SELEX to Select Antisense Padlock Complexes That Can Be Locked to a Target by a Clasp Molecule. Top: Presented is a scheme for selecting antisense complexes that can be locked to a target by a clasp molecule (grey ball). The target DNA, immobilized on a substrate, is mixed with a pool of DNA containing nucleotides randomized in the area shown in bold. Bottom: Example using c-myc as clasp and HER2 as target mRNA. n, sequences to be randomized. “xxx” represents CUU or ethylene glycol residues. In the triplex mode, x would be chosen to pair with the duplex formed by the other two strands, lending additional stability and permitting obstruction of translation. This scheme will adjust the lengths of the duplex and single-stranded linker regions so that there is poor binding without the protein clasp and good binding with it. In this process, the length of duplex required for binding by the truncated c-myc will automatically be selected.

FIG. 10. Inhibition of TNFα Secretion by ATR Constructs. FIG. 10 shows the inhibition of secretion of TNFα by RAW264.7 cells after treatment with control mRNA (m101; i.e., a padlock contruct with an irrelevant antisense region) or the various antisense constructs ATR 1, ATR 16a, ATR 16b or ALR 229. FIG. 11. Gel Shift Analysis on Denaturing Gels of ATR 1, ATR 16a, ATR 16b and ALR 229 Constructs. FIG. 11 shows the results of gel shift analysis on denaturing gels of ATR 1, ATR 16a, ATR 16b and ALR 229 constructs. ATRs were incubated with ³²P-labeled target TNFα RNA fragment (³²P-TT RNA). ATR 1 was used as a negative control because it possesses no antisense region corresponding to the target TNFα mRNA employed.

FIGS. 12A-D. Kinetics of Hybridization and Strong Complex Formation for ATR 1, ATR 16a, ATR 16b and ALR 229 Constructs with TNFα RNA. FIGS. 12A-D show the kinetics of hybridization and strong complex formation for the ATR 1, ATR 16a, ATR 16b and ALR 229 contructs with TNFα RNA target. cc, complementary complex; sc, strong complex.

FIG. 13. Dose Response Curves. FIG. 13 shows dose response curves for ATR 1, ATR 16a and ALR 229 directed against three different target sequences of TNFα RNA in cultured cells.

FIG. 14. Anti-TNFα effects of ALR 229 in Mice. FIG. 14 shows the anti-TNFα effects of ALR 229 in mice, wherein each point represents an average value for 3 mice ±SEM, with TNF assays done in triplicate for each mouse. Transfection reagents were prepared for the RAW264.7 cell assays described below except that the amounts were scaled up so that 10 μg RNA was used per mouse. 1 ml of the resulting liposome:RNA complexes was injected i.p. into mice that had previously been injected with thioglycollate to recruit responsive macrophages. Macrophages were harvested 3 hrs later by peritoneal lavage. The exudates were plated at 1×10⁶/well in 24-well plates, allowed to adhere and recover, then stimulated with LPS and assayed for secretion of TNFα.

FIG. 15. Structural formulas of the platinum reagents. FIG. 15 shows the structural formulas of various platinum reagents, proposed to be used as metal padlocks and for introduction of positive charges into oligonucleotides.

FIG. 16. Proposed mechanism of diethylenetriamine catalysis of chlorotetraplatinate(II) binding to oligonucleotides. FIG. 16 shows a mechanism of diethylenetriamine catalysis of chlorotetraplatinate(II) binding to oligonucleotides.

FIGS. 17A-C. Mechanisms of oligonucleotide platination and structure of platinum adducts. A, diethylenetriamine chelating a chloroplatinate group tethered to a phosphorothioate residue at the sulfur atom. B, diethylenetriamine chelating a chloroplatinate group tethered to the N7 atom of either one or two neighboring guanine residues. C, oligonucleotides labeled with diethylenetriamineplatinum(II) group at different sites.

FIGS. 18A-E. Platination patterns for oligonucleotides to stabilize complementary complexes with single-stranded nucleic acid targets. a, Cationic platinum groups introduced into internal regions of antisense oligonucleotides through the modification of either sulfur atoms of internucleotide phosphorothioates or N7-positions of purine bases. b-e, Cationic platinum groups attached to antisense oligonucleotide constructs through non-hybridizing sequences or linkers by modification of sulfur atoms of internucleotide (and/or terminal) phosphorothioates, or thio-pyrimidines, or thio-containing peptides or other organic oligomers. •—Represents platinum group attached to oligonucleotide prior to hybridization. Each pair of diagrams shows nucleic acid complexes made by both unmodified and platinated oligonucleotides.

FIGS. 19A-E. Platination patterns for oligonucleotides to stabilize triple-helical complexes with either DNA duplexes, or hairpin and single-stranded RNAs. a, “Classic” triplexes with either homopurine or homopyrimidine tfos. b, alternate strand triplexes with oligonucleotides containing both homopurine and homopyrimidine triplex-forming domains. c, triplexes formed by hybrid oligonucleotides containing two triplex-forming domains connected by a non-hybridizing linker. d, oligonucleotides forming both a duplex and a triplex with a target structure containing an internal loop. e, triplex “clamps” featuring hairpin-like structure. •—represents a platinum group attached to oligonucleotide prior to hybridization. Each pair of diagrams shows nucleic acid complexes made by both unmodified and platinated oligonucleotides.

FIG. 20. Cationic platinum derivatives of antisense oligonucleotide constructs designed to open and bind double-stranded regions of DNA and RNA by substituting for competing complementary sequences. An intramolecular self-complementary structures of the antisense oligonucleotides are destabilized by strong ionic repulsion and steric hindrance of the platinum groups. •—Represents platinum group attached to oligonucleotide prior to hybridization. Each pair of diagrams shows nucleic acid complexes made by both unmodified and platinated oligonucleotides.

FIG. 21. Diethylenetriamine catalyzes the platination of oligonucleotides. Autoradiogram, after electrophoresis on 20% denaturing polyacrylamide gel, of [5′-³²P]-labeled TT (lanes 1-11) and TST (lanes 12-22) oligonucleotides and of the products of their modification by the {30 μM K₂PtCl₄+3 mM dien}cocktail in 10×TAE buffer (10 mM Tris-OAc, 1 mM EDTA) for 1 h at 45° C. All samples contained 10 μM of the homologous non-radioactive oligonucleotides to adjust the platinum:oligonucleotide molar ratio to 3:1. Some samples additionally contained KI (lanes 4-7 and 15-18) or NaCl (lanes 8-11 and 19-22) at the indicated concentrations. No K₂PtCl₄ was added to samples 1 and 12. XC and BPB mark the positions in the gel of xylene cyanol and bromophenol blue tracking dyes, respectively.

FIG. 22. Prolonging the incubation time reveals different platination patterns of TT and TST oligonucleotides. Autoradiogram, after electrophoresis on a 20% denaturing polyacrylamide gel of [5′-³²P]-labeled TT (lanes 1-6) and TST (lanes 7-12) oligonucleotides and of the products of their modification by 30 μM K₂PtCl₄ or {K₂PtCl₄+dien}cocktails in 10×TAE buffer after incubation at 45° C. for either 2 h (lanes 3-4 and 9-10) or 4 h (all other lanes). All samples contained 10 μM of the homologous non-radioactive oligonucleotides to adjust the platinum:oligonucleotide molar ratio to 3:1. No K₂PtCl₄ or dien was added to samples 1 and 7.

FIG. 23. Effects of dien concentration and Pt/oligo ratio on the number of products of STT platination. Autoradiogram, after electrophoresis on a 20% denaturing polyacrylamide gel of [5′-³²P]-labeled STT oligonucleotide and of the products of its modification by 30 μM K₂PtCl₄ or (K₂PtCl₄+dien) cocktails in 10×TAE buffer for 2 h at 45° C. Samples contained either 10 μM (lanes 1-5) or 20 μM (lanes 1-5) of the non-radioactive oligonucleotide to adjust the platinum:oligonucleotide molar ratios to 3:1 or 1.5:1, respectively. The concentrations of dien were 1 mM (lanes 2-3) and 3 mM (lanes 4-5 and 9-10). No dien was added to samples 1 and 6. Several samples additionally contained 1 mM KI (lanes 3, 5, 8 and 10).

FIG. 24. Effect of Pt/oligo ratio on platination of STT. Autoradiogram, after electrophoresis on a 20% denaturing polyacrylamide gel of [5′-³²P]-labeled STT oligonucleotide and of the products of its modification by (30 μM K₂PtCl₄+1 mM dien) in 10×TAE buffer for 2 h at 45° C. Concentrations of added non-radioactive oligonucleotide and the corresponding platinum:oligonucleotide molar ratios were as shown. No K₂PtCl₄ was added to samples 1-2.

FIG. 25. Schematic Representation of Binary Recombinant RNA (replicase) Probe Hybridized to the HIV-1 pol RNA Target. The replication probe consists of approximately one-half of the MDV-1 (+) RNA (the template for Qβ replicase when whole) joined at the small arrows to a 12 nt sequence complementary to the target, then one-half of the hairpin ribozyme substrate sequence (7 to 10 nt), and terminating at the ligation site. These replication probes cannot be amplified unless they are hybridized to their target and ligated to the hairpin ribozyme catalytic core (not shown), which is itself folded into the active conformation only in the presence of target. Upon ligation (at the site shown by the arrowhead) the complete molecule is replicated by Qβ replicase in a process that detaches it from the target and ribozyme and may result in its folding into the structure shown on the right. The 40-nt inserted sequence containing the ligation site for the hairpin ribozyme is shown above the small arrows.

FIG. 26. Scheme for Ribozyme-Assisted RNA Amplification Using Qβ Replicase. Presented is a schematic representation of the overall scheme for amplifying nucleic acids using a ribozyme-assisted approach and Qβ replicase.

FIG. 27. Schematic Representation of the Recombinant RNA Capture Probe Bound to a Complementary Sequence of HIV-1 RNA Target. Presented is a schematic representation of the recombinant RNA capture probe (total 60 nt in length) bound to a complementary sequence of HIV-1 RNA target (nt 4577-4760). The 12 nt substrate sequence for the target-dependent hairpin ribozyme (cleavage site shown by arrows) is attached both to the 45-nt hybridization probe through the oligo-U bridge from its 3′-end and to the magnetic bead from the 5′-end. the extended U-bridge is to permit ease of docking with target-bound domain E.

FIG. 28. Scheme for Generating 5′-OH and 2′,3′-Cyclic Phosphate Ends Suitable For Ligation. Presented is a scheme for generating 5′-OH and 2′,3′-cyclic phosphate ends suitable for ligation.

FIG. 29. Sequence Details of the HPR Catalytic Region Stabilized in its Active Conformation by Hybridizing to the Target RNA. Presented is the sequence details of the HPR catalytic region stabilized in its active conformation by hybridizing to the Target RNA. The target sequence is nt 46684682 of the HIV-1 genome. The sequences NNNN connecting domain E with, the target-complementary sequences will be selected so that the catalytic activity of the ribozyme is strictly dependent upon its accurate pairing with the target. On the right is presented constructs employed for selection of the NNNN sequences.

FIGS. 30A-H. Exemplary Schemes for Stabilization of Topological Linkage by Various Means. In A-G, the Watson-Crick non-covalent base pairing is sufficiently weak that the binding of the padlocking or “clasp” molecule is required for stability. In FIGS. 1A-1C, the vertical stem can be determined by selection from random oligonucleotide libraries. A. Presented is a protein binding to a stem structure that may either be a perfect duplex (e.g., for binding of a transcription factor) or a mismatched duplex (e.g., in the case of RNA padlocks and RNA binding proteins such as tat). B. Presented is a small organic molecule binding to a stem structure, wherein the small organic molecule may be naturally occurring in the target cell or introduced therein (e.g., a drug). C. Presented is a metal ion binding to a stem structure, wherein the metal ion may be complexed to natural nucleic acid groups or synthetic features such as P═S groups on phosphorothiolate derivatives and/or sulfur derivatives of bases. D. Presented is a metal clasp without a stem structure, e.g., cis-Pt(NH3)2Cl2 bound to S-substituted termini on the oligo such as phosphorothiolate derivatives or thiolated termini. E. Presented is phosphorothiolate derivatives crosslinked by a Pt-containing agent to bases on a target mRNA (S—Pt-base bonds). F. Presented is crosslinking through S—Pt—S bond formation, where S comes from phosphorothiolate derivative of oligonucleotide or thiolated termini. G. Presented for comparison a covalently closed padlock lacking an external clasp, of which ATR1 is an example. H. Presented is the molecule shown in G before covalent closure.

FIG. 31. Scheme for Using Hammerhead Ribozymes to Detect any Molecule. FIG. 31 presents a scheme for using a hammerhead ribozyme to detect the presence of any molecule (filled oval), which upon encountering the probe, assembles an optomer from dangling ends to which it specifically binds, thereby stabilizing the active conformation of a ribozyme. Subsequent cleavage detected by any of several techniques such as release of biotin from a solid support.

FIG. 32. Scheme for Using Hammerhead Ribozymes to Detect any Nucleic Acid Sequence. FIG. 32 presents a scheme for using a hammerhead ribozyme to detect the presence of any nucleic acid sequence. Cleavage indicates a signaling event such as fluorescence or stimulation of an enzyme.

### DETAILED DESCRIPTION OF THE INVENTION

The present invention is directed to improved antisense and antigene oligonucleotide compositions and methods for down-regulating the expression of various proteins in cells using novel antisense and antigene oligonucleotides which are capable of resisting dissociation from target nucleic acid molecules. More particularly, the present invention is directed to novel antisense and antigene molecules and methods of their use, wherein the novel antisense and antigene molecules are capable of tightly binding to a target nucleic acid not only through standard Watson-Crick pairing (as do standard antisense agents), but also employ additional features that topologically “lock” the antisense or antigene molecule onto the target nucleic acid molecule, thereby making it highly resistant to dissociation promoted by helicases, ribosomes or modifying enzymes and, in turn, imparting improved translation inhibitory properties.

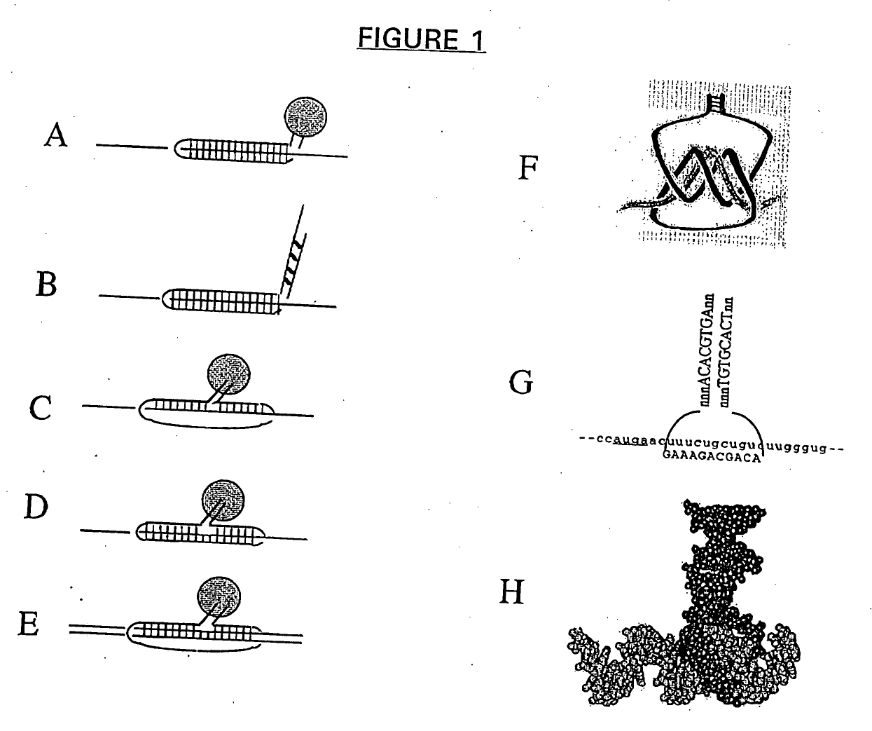

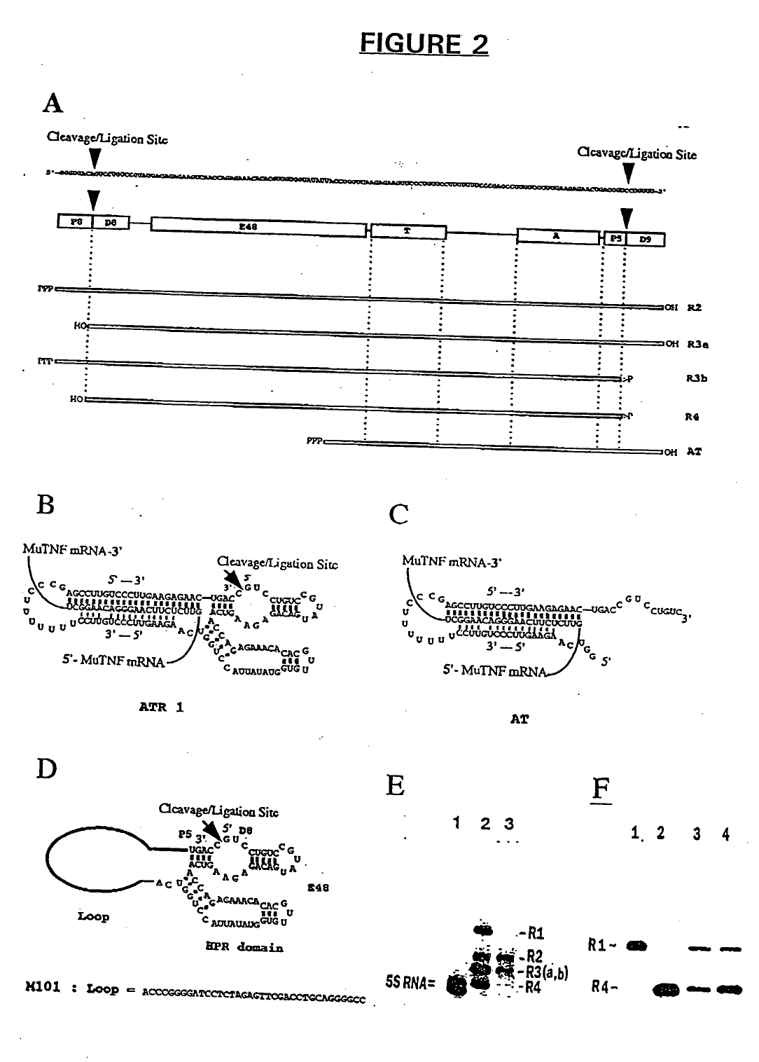

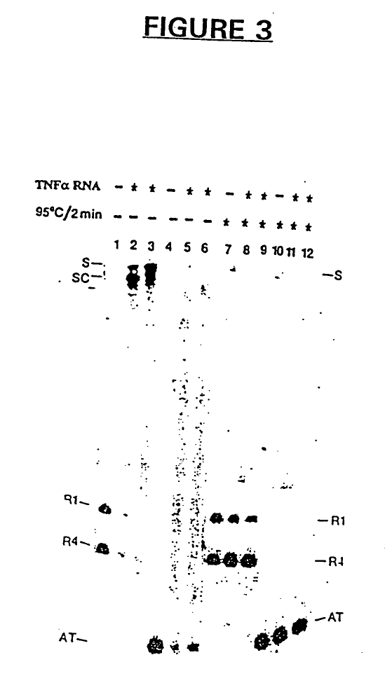

By “topologically linked” is meant that the antisense or antigene oligonucleotide circularizes around the target molecule. For example, if an initially linear antisense or antigene molecule binds to an mRNA target, wraps around it, and then circularizes, it would be very difficult to displace. Unless an endonucleolytic cleavage event occurs in the circular molecule, hydrogen bonds between the two molecules would have to be simultaneously broken, then the mRNA would have to thread its way out of the circle. Although this is theoretically possible, secondary structure in the mRNA would make it kinetically extremely slow. Such molecules are referred to herein as “topologically linked” to the RNA target. FIGS. 1 and 2 illustrate some examples of topological linkage to a target nucleic acid. We refer to molecules capable of such topological linkage as “padlocks”. Topologically linked molecules becomes “locked” or “clamped” onto the target by one or more of a variety of mechanisms described herein, regardless of the structure of the target nucleic acid. The target nucleic acid may be linear, circular or may take any other form that allows topological linkage of the antisense or antigene oligonucleotide thereto. Topologically linked oligonucleotides are not displaced from the target nucleic acid to which they are bound unless (1) the oligonucleotide backbone is broken or (2) by breakage of hydrogen bonds allowing the oligonucleotide to slip off the end of the target nucleic acid. The antisense and antigene molecules of the present invention have sequences that are “substantially complementary” to the target molecule, meaning that those sequences are sufficiently complementary to allow hybridization therebetween via normal base pair binding. Such sequences may be fully complementary or may have one or more mismatch(es). The molecules of the present invention arte either nucleic acids, including both DNA and RNA, as well as analogs thereof. By “analogs thereof” is contemplated nucleic acids containing one or more non-natural or synthetic bases, peptide nucleic acids (PNAs), nucleic acids comprising one or more internucleotide atoms such as sulfur, oxygen nitrogen, and the like.

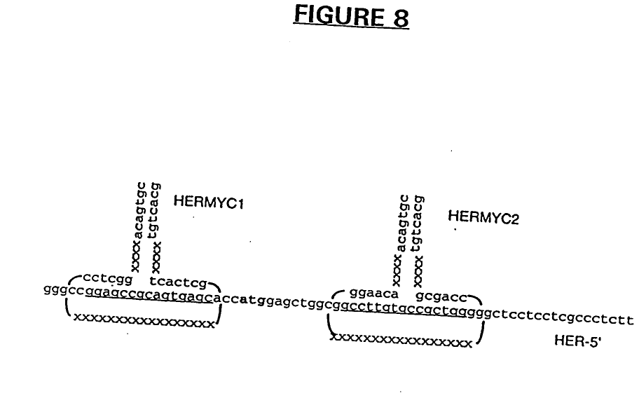

There are various mechanisms by which the novel antisense and antigene molecules of the present invention may become topologically linked to a target nucleic acid molecule. For example, one may employ an antisense RNA to which a catalytic RNA molecule is linked, either through a natural nucleic acid bond or a linking structure. The catalytic RNA molecule is capable of causing the 5′ and 3′ ends of the antisense oligonucleotide to covalently or non-covalently interact with one another, thereby effectively “topologically” linking the antisense molecule to the target nucleic acid. In one embodiment, the catalytic RNA which finds use in the antisense or antigene molecules of the present invention is the hairpin ribozyme which is derived from the minus strand of the satellite RNA associated with tobacco ringspot virus (Buzayan et al., Nature 323:349-353 (1986a), Feldstein et al., Gene 82:51-63 (1989) and Hampel and Tritz, Biochemistry 28:49294933 (1989)). The catalytic domain of the hairpin ribozyme has a compact and stable structure and is capable of autocatalytically cleaving and ligating at a specific site to interconvert between a covalently closed circle and a non-covalently closed form which possesses a 5′OH group and a 2′,3′-cyclophosphate terminus (FIG. 2). As such, when a standard antisense or antigene RNA molecule (i.e., one which generally binds to a target only through standard Watson-Crick base pairing) is modified to contain the catalytic domain of the hairpin ribozyme, the modified antisense RNA is not only capable of recognizing and binding to its target through standard Watson-Crick base pairing and other similar interactions, but also is capable of becoming “locked” onto the target molecule through the catalytic function of the ribozyme. The ribozyme may be catalytically active when the antisense molecule (or nucleic acid encoding it) is introduced into the cell, or may be inactive when introduced and may become activated upon subsequent events which will be described below. Various catalytic RNA molecules are known in the art and may be routinely employed for linkage to an antisense oligonucleotide to facilitate topological linkage to a target nucleic acid. Examples of antisense or antigene constructs that comprise a catalytic RNA are the ATR constructs described below (see FIGS. 2 and 7).

To further stabilize the antisense or antigene molecule/target complex, one can also introduce a triplex-forming region into the antisense or antigene molecule. A triplex-forming region is a nucleic acid sequence which is incorporated into the antisense or antigene molecule and which functions to form a triplex with the duplex that is created between the complementary sequences of the antisense or antigene molecule and its target. While more detail regarding triplex formation and the sequences required therefor is presented below, it is evident to those skilled in the art that the ability to employ triplex-forming sequences will depend upon the sequence of the target nucleic acid and the corresponding antisense or antigene molecule.

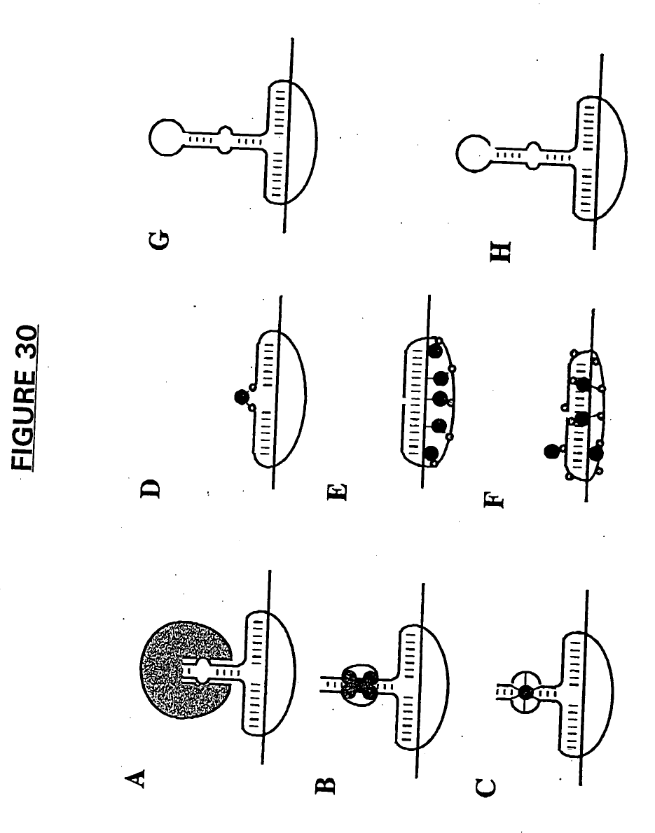

Another mechanism by which the novel antisense or antigene molecules of the present invention may be effectively topologically linked to the target molecule is by incorporating a sequence therein which forms a structure upon specifically binding to the target molecule, wherein the formed structure is subsequently bound by a “locking molecule” which essentially serves as a “clasp” that does not allow the antisense or antigene oligonucleotide to be easy displaced from the target molecule. The sequences which function to serve as the binding site for the “locking molecule” are generally placed at the ends of the antisense or antigene molecule, so as to allow them to interact when the molecule is bound to the target. When these ends interact to form the locking molecule binding site, they often form a structure which is sufficiently weak so as to be unstable. However, when this structure is actually bound by the locking molecule, the structure is stabilized and remains tightly bound to the target molecule. As described above, the locking molecule can be virtually anything which binds to a nucleic acid structure in a sequence- or structure-specific manner including, for example, proteins, nucleic acids, metal ions (either by themselves or complexed to other components such as nucleic acids, and the like), organic or inorganic molecules, drugs, and the like. FIGS. 30A-H provides a schematic illustration of some of these mechanisms. Additional detail for this method will be provided below.

The above described antisense and antigene molecules form a tightly bound complex with the target molecule due to their ability to be topologically linked or circularly fixed around the target. In this regard, there are many advantages for circularly linking an antisense or antigene polynucleotide to a target nucleic acid molecule. For example, circular RNA molecules are generally more stable in the cellular environment than are linear RNA molecules. Linear antisense RNAs, either as in situ transcripts from inserted genes or as ribozymes injected into the body, are particularly susceptible to degradation by nucleases in the cell as well as in extracellular fluids such as blood. However, circularization prevents damage from the most prominent nucleases, which are exonucleases. For example, in vitro studies have demonstrated that the half-life of covalently closed circular oligonucleotides in serum is at least 100 times higher than that for linear oligomers (Nilsson et al., Science 265:2085-2088 (1994)). Moreover, in nuclear and cytoplasmic extracts from HeLa cells, circular ribozyme molecules have better resistance to nuclease degradation than do linear forms of the ribozyme (Puttaraju et al., Nucl. Acids Res. 21:4253-4258 (1993)).

Another advantage to circularly linking an antisense or antigene oligonucleotide to a target nucleic acid molecule is improved strength and specificity of binding as compared to that obtained with linear antisense or antigene oligonucleotides. It has been shown that antisense circles as small as 30 to 40 nucleotides can form regular 15-18 bp duplexes with target sequences in mRNAs (Dolinnaya et al., Nucl. Acids Res. 21:5403-5407 (1993)). In the case of DNA, circular oligodeoxyribonucleotides have been shown to bind effectively to single-stranded homopurine or homopyrimidine nucleic acids by triplex formation (Kool, J. Amer. Chem. Soc. 113:6265-6266 (1991)). They show higher sequence selectivity and less tolerance of mismatches with target sequences than do ordinary linear oligomers (Kool (1991), supra, Prakash and Kool, J. Chem. Soc. Chem. Commun. 1161-1163 (1991), Prakash and Kool, J. Am. Chem. Soc. 114:3523-3527 (1992), Wang and Kool, Nucl. Acid. Res. 22:2326-2333 (1994)). A comparison of circular RNA and DNA oligonucleotides of the same sequence showed that RNA circles bind single-stranded RNAs with considerably higher affinity than do DNA circles, even without topological linkage of the oligonucleotides to the targets as described herein (Wang and Kool (1994), supra).

Small RNA molecules are also advantageous as antisense and antigene agents because they minimize possibilities for folding into alternate conformations that can interfere with target recognition (Forster and Symons, Cell 50:9-16 (1987), Hélène and Toulmé, Biochim. Biophys. Acta 1049:99-125 (19900 and Dolinnaya et al. (1993), supra). However, for in situ expression, additional sequences are needed for high-levels or cell-type-specific expression. If a circular RNA is to be the active antisense or antigene agent for the reasons described above, the best way to lessen conformational problems is to autocatalytically excise from the final circle any sequences that are irrelevant for target binding.

We consider herein that the hairpin ribozyme is appropriate for topologically linking an antisense or antigene oligonucleotide to a target nucleic acid because of the compact and stable structure of its catalytic domain (Feldstein and Bruening, Nucl. Acids Res. 21:1991-1998 (1993), Anderson et al., Nucl. Acids. Res. 22:1096-1100 (1994) and Butcher and Burke, J. Mol. Biol. 244:52-63 (1994)) and its high catalytic activity in experiments both in vitro and in vivo (Yu et al., Proc. Natl. Acad. Sci. USA 90:6340-6344 (1993), Chowrira et al., J. Biol. Chem. 268:25856-25864 (1994)). The hairpin ribozyme is derived from the minus strand of the satellite RNA associated with tobacco ringspot virus. The precursor RNA is synthesized in an infected cell as a multimer that self-cleaves to a monomeric unit. The monomeric form freely interconverts between a covalently closed circle and a noncovalently closed form containing a 5′-hydroxyl and a 2′,3′-cyclophosphate terminus. Autocatalytic cleavage and ligation occurs at a specific site (see FIG. 2B, below).

The hairpin ribozyme can be separated into catalytic, substrate, and substrate-binding moieties. Mutagenesis, deletion analysis, chemical structure mapping, and in vitro selection experiments have identified a minimal 48-nt sequence, herein termed E48 or the minimonomer, and secondary structure requirements essential for the hairpin ribozyme autocatalytic function (Hampel et al., Nucl. Acid Res. 18:299-304 (1990), Feldstein and Bruening (1993), supra, Anderson et al. (1994), supra and Butcher and Burke (1994), supra) (see FIG. 2B, below). The complex between ribozyme and substrate sequences is stabilized by several factors, including two helices that flank a symmetrical internal loop (loop LA; see FIG. 2D) near the cleavage/ligation site, as well as intraloop (within loop LA) and interloop (between loops LA and LB) specific noncanonical H-bonding (Berzal-Herranz et al., EMBO J. 12:2567-2574 (1992) and Butcher and Burke (1994), supra). The structure and the sequence of the junctions between the substrate and enzyme parts of the hairpin ribozyme can be used for substitutions or insertions.

Triplexes: The most common pairing motif for nucleic acid triplexes consists of a pyrimidine third strand pairing with a Watson-Crick duplex, where thymine recognizes A:T base pairs, protonated cytosines recognize G:C base pairs and the third strand is parallel to the purine strand of the duplex. We have recently demonstrated (Jayasena and Johnston, Biochemistry 31:320-327 (1992a), Jayasena and Johnston, Nucl. Acids Res. 20:5279-5288 (1992b) and Jayasena and Johnston, Biochemistry 32:2800-2807 (1993a)) that this motif can be combined with a second, purine third-strand motif to permit triplex formation at sequences other than pure homopurine-homopyrimidine blocks. This capability will permit the antisense- or antigene-triplex approach proposed herein to be applicable to a greater variety of target sequences than would otherwise have been possible.

Triplex formation may require protonation of cytosines in the third strand. Because the pK_{A} of cytosine is shifted substantially toward neutral in a triplex, the triplexes proposed are expected to be stable under physiological conditions. However, if triplex stability is limiting, constructs will be synthesized with 5-methyl cytosine in place of cytosine at the third strand positions, which shifts the pK to higher values.

Any gene in which at least a portion of the coding and/or non-coding sequence is known or readily obtainable and which would benefit from a lower expression thereof will serve as targets for the novel antisense and antigene molecules of the present invention. As will be apparent from the present disclosure, the antisense and antigene oligonucleotides of the present invention can be routinely adapted to bind to and become topologically linked to virtually any target nucleic acid molecule of interest. As such, the presently described antisense molecules represent a major step forward in the field of antisense therapeutics.

Delivery into Cells: Previous studies have demonstrated that catalytic RNA can be delivered to cells utilizing cationic lipids, such as N[1-(2,3-dioleyloxy)propyl]-N,N,N-trimethylammonium chloride (DOTMA) (Sioud et al., J. Mol. Biol. 242:831-835 (1991)). Other investigators (Zhu et al., Science 261:209-211 (1993)) have used a 1:1 ratio of DOTMA with dioleoylphosphatidylethanolamine (DOPE) to achieve systemic expression of plasmid DNA following intravenous injection of plasmid DNA:DOTMA:DOPE complex into mice. Other cationic lipid formulations have become commercially available (e.g. DOSPA:DOPE, DOTAP, DMRIE:cholesterol, DDAB:DOPE, and others) and offer improved expression of DNA. A 1:1 mixture of dioctadecylamidoglycylspermine (DOGS):DOPE is effective to introduce ribozyme constructs into cells both in vivo and in vivo (see, e.g., Kisich and Erickson, J. Leukocyte Biol. Suppl. 2:70 (abstract) (1991a) and Kisich and Erickson, FASEB J. 4:1860 (abstract) (199 lb)). As presented below, 1:1 DOSPA:DOPE (Lipofectamine, Life Technologies, Inc.) is shown to be effective for introducing padlock RNAs into mouse macrophages in vitro and in vivo.

The antisense and antigene molecules of the present invention will find use for reducing or inhibiting expression of a target gene both in vitro and in vivo. For administration, the antisense and antigene molecules may be directly administered by various techniques which are known in the art including transfection, transformation, infection, and the like. Additionally, expression constructs may be employed to provide an expression template (inducible or not) that can provide a continuous source of the antisense or antigene molecule of interest to the cell(s). As such, gene therapy methods are encompassed within the present invention. Vehicles for introducing and inducing expression of introduced nucleic acids are well known in the art and may be readily employed herein.

We also herein describe novel methods for platination of antisense or antigene oligonucleotides and triplex forming oligonucleotides (TFO) through either selective modification of phosphorothioate (POS) linkages in homopyrimidine sequences or guanine residues in purine-rich oligonucleotides.

Effect of metals and polyamines on triplex formation. The fact that triple-stranded nucleic acid structures are usually less stable than related duplexes, particularly if the third strand has backbone modifications such as phosphorothioate substitutions, is a factor limiting the use of the TFOs as antigene agents (Wilson et al., 1993; Lacoste et al., 1997). The lower stability of nucleic acid triple-helical interactions is caused, at least in part, by the added electrostatic repulsion of the third chain relative to duplexes. Moreover, most triplexes employing pyrimidine-rich TFOs are not stable at physiological pH (due to need for cytosine protonation), and physiological concentrations of K⁺ may hamper triplex formation with purine-rich TFO's (Thuong and Helene, 1993). The negative effect of K⁺ on a purine-purine-pyrimidine triplex formation is presumably due to self-association of the oligonucleotides in competitive structures such as parallel duplexes (GA-rich TFOs) and/or tetraplexes (GGGG-containing TFOs) (Musso and Van Dyke, 1995; Olivas and Maher, 1995a; Lacoste et al., 1997). Some mono-, di- and multivalent metal cations (Malkov et al. 1993; Thuong and Helene, 1993; Kazakov, 1996; Ellouze et al., 1997), as well as cationic polyamines (Thomas and Thomas, 1993; Musso and Van Dyke, 1995; Pallan and Ganesh, 1996) favor triplex (both pyrimidine- and purine-rich) formation.

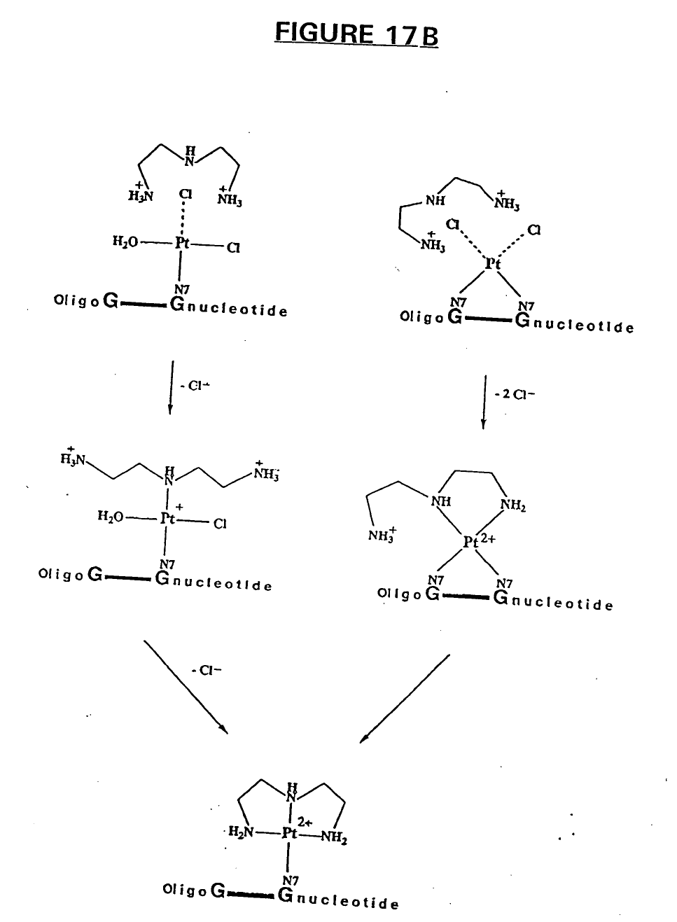

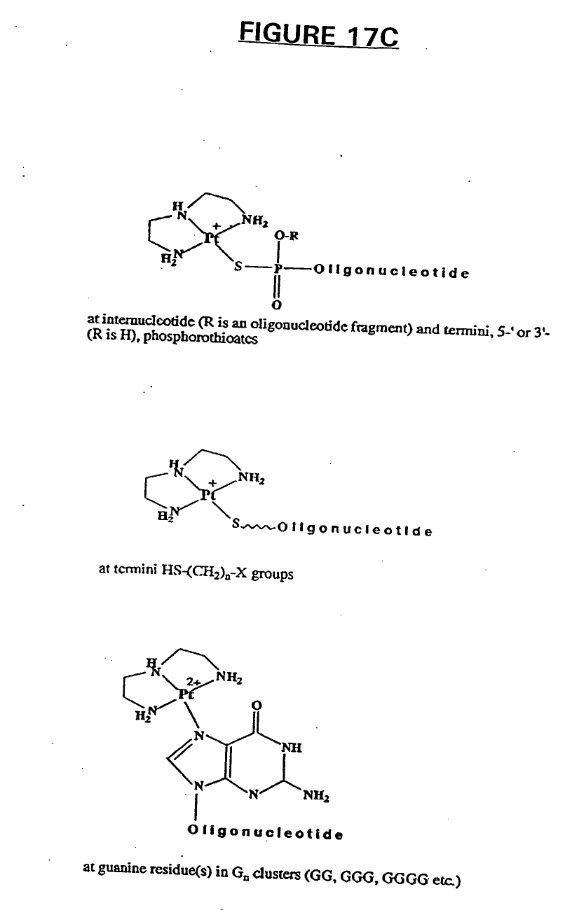

However, the inhibitory effect of K⁺ can be completely overcome or reversed by physiological concentrations of such favorable cofactors as Mg²⁺, spermine⁴⁺ or spermidine³⁺ (Musso and Van Dyke, 1995; Olivas and Maher, 1995a). Approaches to destabilizing aggregates of purine-rich TFOs under physiological conditions would aid their biological applications (Olivas and Maher, 1995a; Svinarchuk et al., 1996). A remarkable solution for this problem has been accomplished by using 6-thioguanine substituted for guanine, presumably because the increased radius and decreased H-bonding ability of sulfur in the C6-position destabilize potential guanine tetraplexes (Olivas and Maher, 1995b). We propose that platination of the guanine N7-position (see FIG. 17B), which also plays a key role in tetraplex formation through H-bonding, could result in a similar suppressing effect on guanine quartet-mediated aggregation of TFOs.

Attachment of functional groups to triplex-forming and antisense oligonucleotides. The enhancement of triplex stability by cations can be further exploited by their conjugation with the TFOs. For example, the attachment of different cationic peptides (Tung et al., 1996) and polyamines such as spermine (Tung et al., 1993) to the 5′-end of the homopyrimidine oligonucleotides boosts the stability of triplexes while having no affect on the stability of the underlying double helix.

Here, we propose the attachment of conjugates of platinum and polyamine cations to TFOs. Currently, there is no information available about the effect on triplex stability of the of platinum complexes tethered to TFOs. However, it is reasonable to expect that appropriate “platination” of oligonucleotides will not compromise their ability to form triplexes, and might even have positive effects. For example, some other metal complexes, [Fe²⁺-EDTA] and [Cu⁺-phenanthroline], attached to oligonucleotides at either their termini, bases or sugar moieties, have been successfully used as chemical probes of triplex structures (Moser and Dervan, 1987; Francois et al., 1989; Beal and Dervan, 1992; Jayasena and Johnston, 1992; Thuong and Helene, 1993; Shimizu et al., 1994; Tsukahara et al., 1996).

Covalent attachment of other functional groups to the oligonucleotides has been employed to alter their affinity for both double- and single-stranded complimentary nucleic acids as well as for introducing reporter groups for structure analysis, providing non-radioactive labels, or preparing synthetic “exonucleases” (see for review: Helene 1993; Thuong and Helene 1993; Plum and Pilch, 1995; O'Donnel and McLaughlin, 1996; Haner and Hall, 1997). Many chemical and biochemical approaches can be used for introducing additional functional groups into commercially available, deprotected oligonucleotides or their derivatives (see for review: Thuong and Helene, 1993; O'Donnel and McLaughlin, 1996). Among these, the functionalization of termini and internucleotide phosphorothioate groups offers a number of advantages over the modification of nucleoside residues (Chu and Orgel, 1994; Fidanza et al., 1994; O'Donnel and McLaughlin, 1996). One advantage is that the attachment of a functional group or label at such sites should not drastically alter the stability of nucleic acid complexes. The alkylation of sulfur in terminal phosphorothioates by substituted alkyl halides was extensively used for functionalization or/and labeling of both antisense and triplex-forming oligonucleotides (Thuong and Helene, 1993; Chu and Orgel, 1994; Shimizu et al., 1994; Tavitian et al., 1998).

The internucleotide phosphorothioate diesters are not as nucleophilic as terminal phosphorothioate esters or alkyl thiols (O'Donnel and McLaughlin, 1996). However, reactive groups such as haloacetamides, azirinylsulfonamides, γ-bromo-α,β-unsaturated carbonyl, and monobromobimane (O'Donnel and McLaughlin, 1996), as well as divalent mercury and platinum (see below) can be used to modify the thioester, forming covalent adducts which are stable under neutral and acidic conditions but can undergo hydrolysis at high pH. An important feature of this approach is the precise placement of the functional group according to the position of the individual phosphorothioate in the synthetic oligonucleotide (Ozaki and McLaughlin, 1992). Moreover, the modification of every internucleotide POS allows the incorporation of multiple groups; ideally one for each POS residue (Conway et al., 1989; Hodges et al., 1989; Conway and McLaughlin, 1991). The nucleophilicity of sulfur in phosphorothioate oligonucleotides has also been used to conjugate them with platinum reagents (see below).

Specific platinum labeling of phosphorothioate nucleic acids. Platinum (and mercury) compounds can specifically bind to sulfur atoms either occurring naturally (e.g., in tRNA molecules) or synthetically incorporated in nucleic acids (Pal et al., 1972; Jones et al., 1973; Scheit and Faerber, 1973; Strothkamp and Lippard, 1976; Strothkamp et al., 1978; Szalda et al., 1979; Chu and Orgel, 1989, 1990, 1991, 1992; Elmroth and Lippard, 1994; Slavin et al., 1994). The resulting modified nucleic acids are potentially useful for X-ray crystallography, electron microscopy or other applications requiring heavy metal labeling (Strothkamp and Lippard, 1976; Lippard, 1978; Strothkamp et al., 1978; Szalda et al., 1979), as well as antisense and antigene probes (Chu and Orgel, 1989, 1990, 1991, 1992).

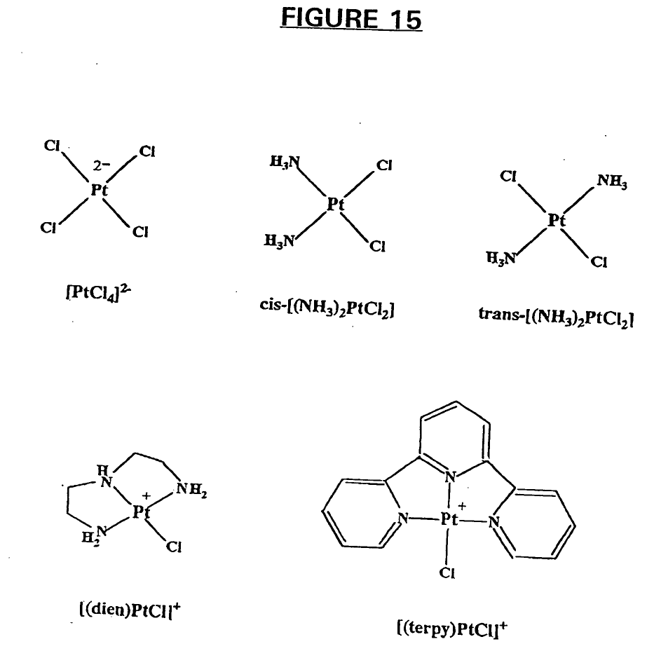

However, little quantitative information is available about the reactivity of phosphorothioates in nucleic acids toward platinum reagents, and only a few kinds of such reagents have been studied so far. One of them is [(terpy)Pt^{II}x]ₙ+(FIG. 15), which has demonstrated almost quantitative binding to the sulfur atoms in nucleoside monophosphorothioates (AMPS and UMPS) and double-stranded poly(_{S}A-U) (Strothkamp and Lippard, 1976) as well as in yeast tRNA^{Phe} containing a modified C_{S}-C_{S}-A at the 3′-end (Szalda et al., 1979). Reactions between 4-40 μM of this platinum reagent with r_{f} (platinum to nucleic acid molar ratio) in the range 0.005 to 5 were performed in [50 mM Tris-HCl (pH 7.5), 0.1 M NaCl] buffer at 25° C. for 10 min. Cation exchange column chromatography on AG50W-X8 (Bio-Rad) was successfully used to remove all noncovalently bound [terpy)PtCl]⁺. There was no evidence for loss of platinum from, or degradation of, phosphorothioate linkages in the purified platinated polyribonucleotides. The data showed that the platinum reagent binds selectively to the phosphorothioate groups in these polyribonucleotides even if the platinum reagent was in excess since no platinum binding to corresponding all-phosphodiester RNA was found under the same conditions (Strothkamp and Lippard, 1976). Binding to the C_{S}-C_{S}-A-modified tRNA molecule was complete with the attachment of two platinum complexes, one for each phosphorothioate group. Even extended incubation periods of up to 24 h resulted in no additional binding of [(terpy)Pt] moiety per the tRNA molecule at the 1>r_{f}>5 (Szalda et al., 1979).

Because of the polyelectrolyte effect (Gueron and Weibuch, 1981; Elmroth and Lippard, 1994), cations and H-bond donors are selectively attracted to the negatively charged surface of the oligo- and polynucleotides. Consequently, high local concentration in the vicinity of the polymer molecules, aggregation on the polymer surface and high mobility along the polyanion backbone may significantly enhance the reactivity of attracted cationic reagents. Conversely, the reactivity of anionic reagents towards polyanions should be significantly less in comparison to reactivity with monomers or neutrally charged polymers, and should be enhanced for positively charged polymers. Elmroth and Lippard (1994) showed that such polymer surface effects provide approximately 25-fold higher rate for formation of the Pt—S linkage with d(TTTTTTT_{S}TTTTTTT) in comparison to platination of the dinucleoside monophosphate d(T_{S}T) by cis-[Pt(NH₃)(NH₂C₆H₁₁)Cl(H₂O)]⁺ (an analog of the reactive form of the well known anticancer agent cis-[Pt(NH₃)₂Cl₂] (FIG. 15)). No difference between reactivity of this reagent toward single-stranded phosphorothioate-containing hexadecaoligonucleotide and the corresponding oligonucleotide duplex was observed.

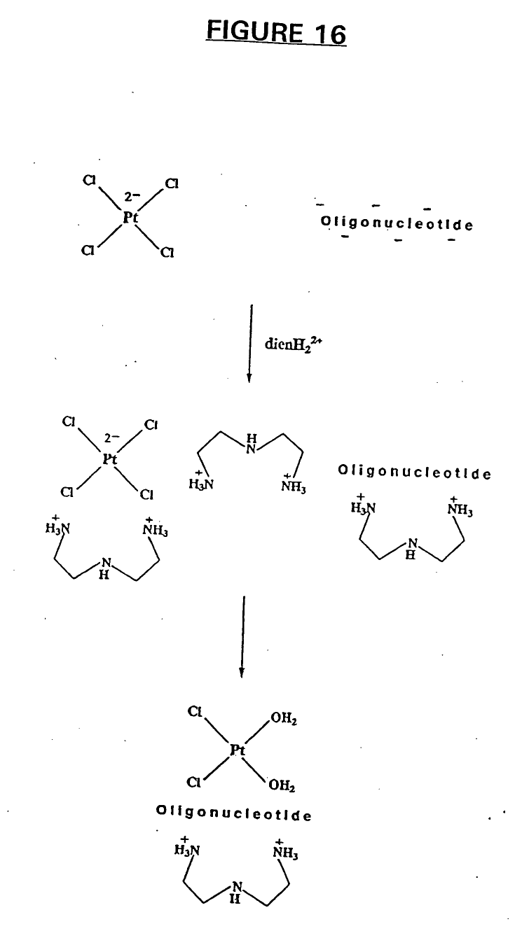

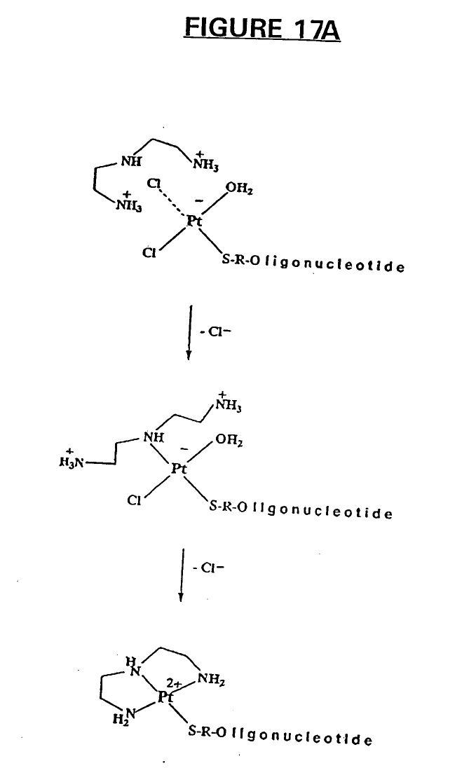

Orgel and co-workers used oligonucleotides containing phosphorothioate and cystamine groups for specific crosslinking of DNA/RNA duplexes (Chu and Orgel, 1989, 1990a; 1990b), DNA triplexes (Gruff and Orgel, 1991) and DNA-protein complexes (Chu and Orgel, 1992) by different platinum reagents. The crosslinking experiments were performed in buffer solutions containing 30-50 mM NaClO₄, 1-7 mM Na-phosphate (pH 7-7.4) and 0.025-0.1 mM EDTA at room temperature overnight in the presence of a large excess of platinum reagent (1-5 μM) over oligonucleotide derivatives (18-72 nM) (Chu and Orgel, 1989; 1990a). The authors could not determine the yield and nature of platinum complexes with POS oligonucleotides because these complexes did not give sharp bands on electrophoresis but they noticed that the crosslinking occurred much more efficiently with phosphorothioate oligonucleotides than with normal all-phosphodiester oligonucleotides (Chu and Orgel, 1989, 1992). However, they noticed higher reactivity of positively or neutrally charged cis-/trans-[(NH₃)₂PtCl₂] (and products of their hydrolysis) than negatively charged K₂[PtCl₄] (Chu and Orgel, 1990a). Chu and Orgel (1990a) have also discussed the possibility of intramolecular crosslinking into phosphorothioate oligonucleotides by K₂[PtCl₄] having four reactive Pt—Cl coordinates, noticing that such crosslinking products (if they were formed) were not stable and underwent further chemical transformation (Chu and Orgel, 1990a). Diethylenetriamine Catalyzes Platination of Oligonucleotides. In aqueous solutions, [PtCl₄]²⁻ (FIG. 15) is known to react very slowly with either polynucleotides alone (Wherland et al., 1973; Chu and Orgel, 1989; Kasianenko et al., 1995) or diethylenetriamine (dien) alone (FIG. 16) (Watt and Cude, 1968; Mahal and Van Eldik, 1987), forming complex mixtures of products in both cases. We found, that in the three-component mixtures, oligonucleotide platination in the presence of dien proceeds rapidly (<2 h at 45° C.) and with a high yield of homogeneous products even at low, micromolar platinum concentrations (10-30 μM). To prepare 50-100 pmoles of the platinum oligonucleotide derivatives, only 0.3 nmoles of the platinum reagent is required. Positively charged dienH₂₂+presumably counteracts the electrostatic repulsion between [PtCl₄]²⁻ and polyanionic phosphate backbone, bringing these two together in very close proximity and stimulating initial platinum binding to oligonucleotide (FIG. 16). Subsequently, the oligonucleotide accelerates chelation of the tethered platinum by dien due to preassociation of the cationic polyamine with the negatively charged nucleic acid surface. The final reaction products presumably consist of diethylenetriaminoplatinum(II), [dienPt]²⁺, forming chemically inert and thermodynamically stable adducts through sulfur of POS (FIG. 17A) or the N7 of guanine residues (FIG. 17B). These adducts are compact and carry a positive charge with potential to promote hybridization to other nucleic acids. (Lepre and Lippard, 1990). The additional positive charge of the platinum groups also allows easy separation of platinated oligonucleotides from reaction mixtures by either preparative electrophoresis (as used in this work) or HPLC. We also consider metallo-affinity chromatography as an alternative, one-step method of purification of the platinated phosphorothioate oligonucleotides.

The major advantages of this new synthetic method are related to the labeling of oligonucleotide probes by radioactive molecules. This is a one-tube reaction which can be performed in any biochemical lab. No special equipment or skills for multiple steps, radioactive synthesis of platinum compounds and product isolation procedures are required (see for comparison Hoeschele et al., 1980; Anand and Wolf, 1992; Azure et al., 1992). Because of the comparatively fast decay of the radioactive platinum isotopes ^{193m} Pt and ^{195m}Pt [t_{1/2} about 4 days (Stepanek et al., 1996)], the short overall time of the oligonucleotide labeling (a few hours, including purification) is very important. In contrast, long (from overnight to several days) multi-step synthetic procedures are required for preparation of alternative platinum reagents such as [(dien)PtCl]Cl (FIG. 15) (Mahal and Van Eldik, 1987) and [(terpy)PtCl]Cl (FIG. 15) [(Strothkamp and Lippard, 1976) which could be potentially useful for oligonucleotide labeling. Because of the high cost of radioactive platinum production, microscale and high-yield synthetic procedures are required for preparation of radioactive platinum reagents. Unfortunately, the inorganic synthesis of platinum reagents suitable for oligonucleotide labeling are not optimized for microscale procedures. Usually, the yields for microscale synthesis are not as high as reported for larger scale procedures (Azure et al., 1992). The exception is the multi-step, semi-automated synthesis of [^{195m}Pt]cis-[(NH₃)₂PtCl₂] which requires at least 8 hrs. (Anand and Wolf, 1992). But cis-[(NH₃)₂PtCl₂] (FIG. 15) is not suitable for specific oligonucleotide labeling because of its tendency to form a mixture of intramolecular crosslinking products which destabilize nucleic acid complexes (Sherman and Lippard, 1987; Lepre and Lippard, 1990). Phosphorothioate (POS) analogues of nucleic acids. Phosphorothioate (POS) analogues of nucleic acids have sulfur in place of non-bridging oxygens bonded to phosphorus in terminal or internucleotide phosphates (see for review Eckstein, 1983; and Zon and Stec, 1991). Phosphorothioate oligonucleotides can be constructed with the P—S residue(s) at selected positions or throughout the entire phosphate backbone. The backbone modification leads to unique physicochemical (see below), chemical (see below) and biochemical features for phosphorothioate oligonucleotides, including: chirality at the phosphorus atom, producing so-called Rp and Sp stereoisomers; greater nucleophilicity and affinity towards heavy metals (see below); resistance to enzymatic cleavage in vivo; and a convenient radiolabeling using ³⁵S isotope. ³⁵S-labeling (and also radioactive platinum labeling) allows control over phosphorothioate oligonucleotide concentration and distribution both in vitro and in vivo. These molecular features have been already utilized for diverse biomedical, biochemical and biophysical applications such as antisense and antigene technology, heavy atom labeling of nucleic acids for electron microscopy, metallo-affinity chromatography of nucleic acids, catalytic RNA (ribozymes), enzyme biochemistry, nucleic acid-protein interactions, and oligonucleotide-directed mutagenesis.

The fact that phosphorothioate oligonucleotides have already been subjected through extensive biological tests would make it easy to repeat similar studies using the platinated analogs.

Preparation and Purification Methods of Phosphorothioates. A terminal phosphorothioate can be easily attached to the 5′-end of both RNA and DNA of unmodified oligonucleotides by polynucleotide kinase and ATPyS, and the 3′-end of RNA (but not DNA) can be phosphorothioated by RNA ligase and dpCp(S) (Eckstein, 1985).

The non-bridging phosphorothioates can be incorporated into the backbone of nucleic acids by chemical or enzymatic methods (Zon and Stec, 1991). Effective analytical and preparative chromatography (reverse phase HPLC) methods for purification and analysis of phosphorothioate oligonucleotides were developed for their clinical evaluation as antisense agents (Zon and Geiser, 1991; Zon and Stec, 1991; Padmapriya et al., 1994; Gerstner et al., 1995). Due to the present unavailability of a chemical stereo-specific synthesis, synthetic oligonucleotides with n phosphorothioate residues are mixtures of 2ⁿ possible diastereisomers (Rₚ and Sₚ). Only the stereoisomers of oligonucleotides with a few phosphorothioate residues can be separated and purified by HPLC (Chu and Orgel, 1990). Enzymatic synthesis of phosphorothioate polynucleotides (RNA or DNA) using appropriate templates, polymerases, and thiotriphosphate nucleotides yields stereo-specifically the Rₚ isomers. The purification protocols of phosphorothioate polynucleotides longer than 50-mers are usually based on metal-affinity, chromatography, and electrophoresis (see below).

Phosphorothioate physico-chemical properties. Replacing an oxygen by sulfur in a phosphate reduces the charge on the remaining oxygens while increasing the negative charge on sulfur. Protonation of phosphorothioates occurs preferentially on oxygen rather than sulfur since phosphorothioates are stronger acids (have lower proton affinities) than phosphates (Frey and Sammons, 1985; Liang and Allen, 1987).

The charge polarization (O=P—S⁻) and diastereoisomer linkages in phosphorothioates could have implications for structures and stability of their complexes, both antisense and triplex (Frey and Sammons, 1985). Furthermore, the larger sulfur atom and longer P—S bond (in comparison to P—O) could lead to steric crowding, especially in triplexes (Latimer et al., 1989; Hacia et al., 1994). Also, since sulfur forms weaker hydrogen bonds than oxygen, the phosphorothioate modification could disturb or modify specific H-bonding interactions with the first water shell in the major groove of duplexes. (Hacia et al., 1994). Biophysical studies demonstrate that oligonucleotides exclusively containing all −Rₚ or all −Sₚ, or random diastereoisomer mixtures of POS linkages, have different affinities for complementary single- and double-stranded sequences (Kim et al., 1992; Hacia et al., 1994; Lacoste et al., 1997). DNA duplexes formed by phosphorothioate (POS) oligonucleotide derivatives are usually less stable than those made of unmodified oligonucleotides (Latimer et al., 1989; Kibler-Herzog et al., 1991; Jaroszewski et al., 1992; Kanehara et al., 1995; Hashem et al., 1998), depending on the number of POS linkages, and their stereochemistry and location in the DNA sequence. For example, in the duplex, [d(GGsAATTCC)]₂, the “inward” oriented (when S atom points into the major grove) Rₚ phosphorothioate isomer had a Tₘ only 1° C. below that of the all-phosphodiester duplex, while the “outward” oriented Sₚ isomers had almost no effect on Tₘ (Zon and Geiser, 1991). The lower binding affinity of short (<15 nt) all-POS antisense oligonucleotides can be offset simply by extending their complementary sequences by several nucleotides (Zon and Geiser, 1991).

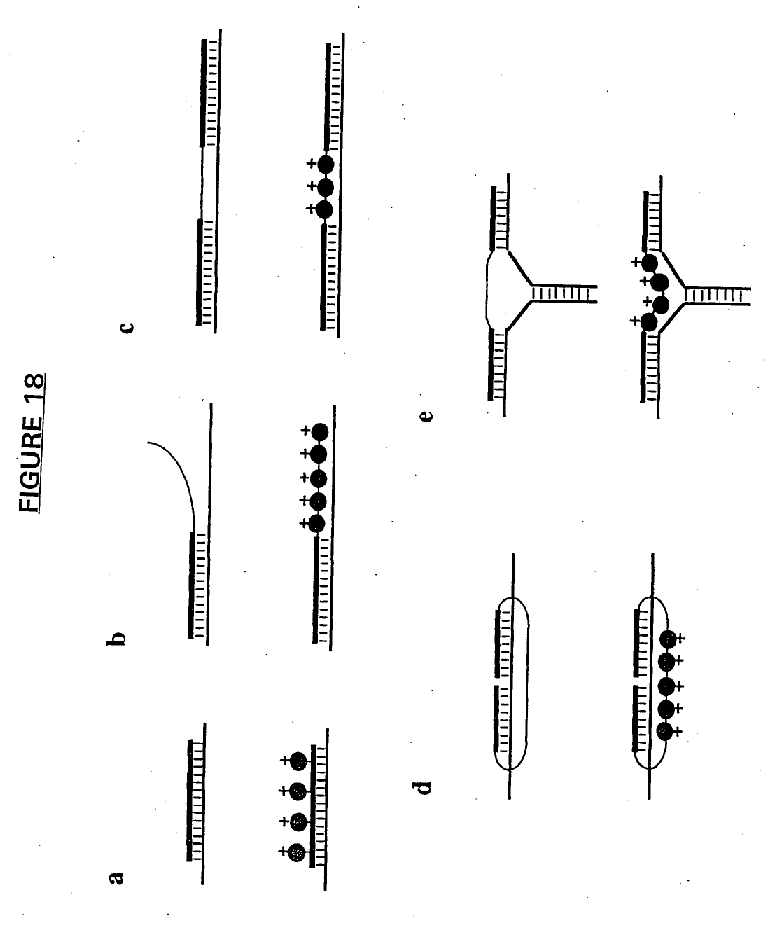

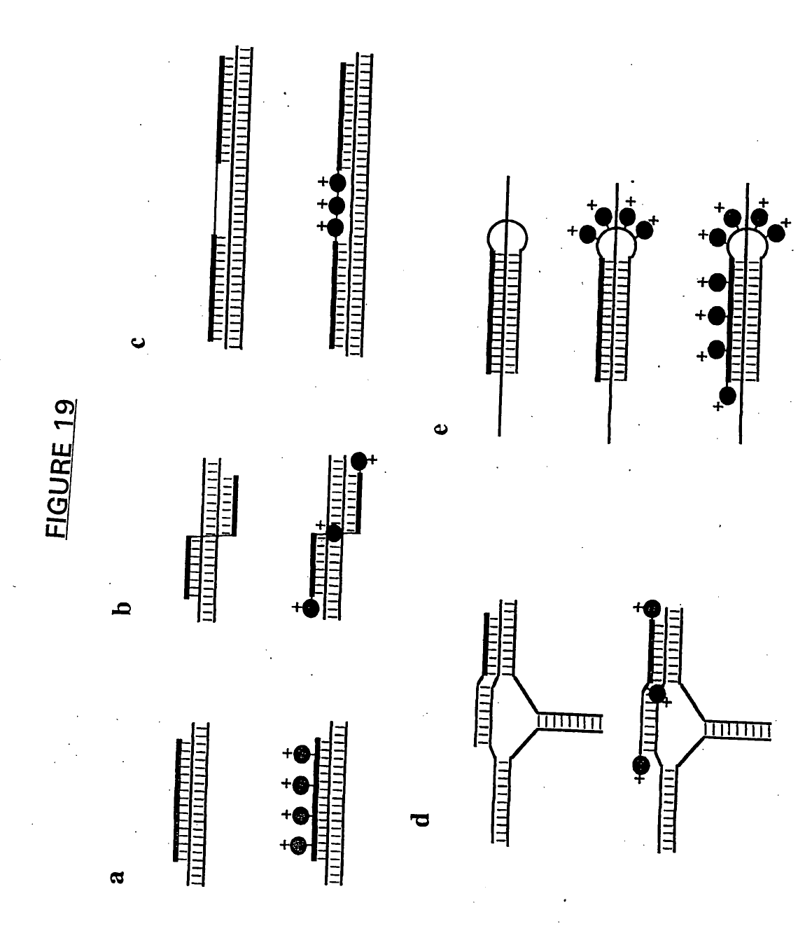

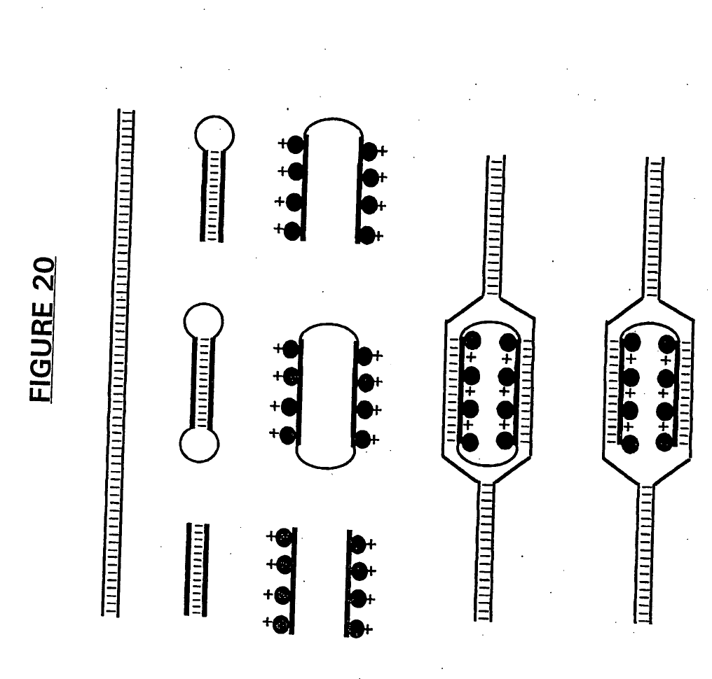

In triplex-forming oligonucleotides (TFO), a small number of POS linkages at or near the ends (see FIG. 19) do not significantly destabilize triple-helical complexes formed by either purine—(Lacoste et al., 1997) or pyrimidine-rich oligodeoxynucleotides (Kim et al., 1992; Alumni-Fabbroni et al., 1994; Xodo et al., 1994; Tsukahara et al., 1993, 1996, 1997). Also, homopurine (G and A-rich) oligonucleotides with all-POS linkages showed no significant reduction of the binding affinity to complementary duplexes (Latimer et al., 1989; Musso and Van Dyke, 1995; Joseph et al., 1997; Lacoste et al., 1997) or even provided a modest increase in the stability (Latimer et al., 1989; Hacia et al., 1994; Musso and Van Dyke, 1995) depending on their sequences. In contrast, all-POS homopyrimidine (Kim et al., 1992; Hacia et al., 1994) and GT-containing oligonucleotides (Lacoste et al., 1997) bind target duplex DNA with drastically reduced affinities in comparison with relative all-phosphodiester oligonucleotides (Kim et al., 1992; Hacia et al., 1994). In general, the binding energy of triplex formation for derivatives of pyrimidine TFOs decreased with the number of POS linkages (Lacoste et al., 1997). However, pyrimidine TFOs containing up to 20% POS linkages can repress a transcription with efficiency comparable to that of all-phosphodiester oligonucleotides (Alumni-Fabbroni et al., 1994).

In fact, because they have high resistance towards exonucleases, oligodeoxynucleotides with POS-capped ends should be superior to normal oligonucleotides for experiments in vivo (Alumni-Fabbroni et al., 1994).

Relatively new bifunctional oligonucleotide probes, combining antisense and triplex-forming domains (see FIGS. 19D and E), allow specific targeting of single-stranded and hairpin regions in mRNAs (Brosalina et al., 1993; Kandimalla et al., 1995; Francois and Helene, 1995; Moses and Schepartz, 1996). Such TFOs, as well as oligonucleotides recognizing DNA by alternate strand triple helix formation (Beal and Dervan, 1992; Jayasena and Johnston, 1992) and DNA's containing two TFO domains connected by a flexible linker, (Kessler et al., 1993) have convenient sites between these domains for introducing POS linkages or reactive, nonhybridizing nucleotide sequences (see FIGS. 19B and 19C). We believe these sites seem to be most appropriate for chemical post-modification (e.g., by platinum reagents) without damaging the ability of these oligonucleotides to form specific complexes with nucleic acids targets. Recently, it was shown that the phosphorothioate internucleotide linkages inside a loop of the parallel-stranded hairpin complexes (see FIG. 19E) do not affect the stability of the triple helix complexes (Tsukahara et al., 1997). However, no other information is currently available on this topic.

Reactivity of the phosphorothioates versus purines in nucleic acids. Although both the N7- and N1-positions of adenosine are known to bind platinum, Strothkamp and Lippard (1976) showed that in the reaction between [(terpy)PtX]ₙ+ and AMPS at r_{f}≦1, binding occurred exclusively at sulfur. In contrast to the experiments with polynucleotides, reaction at the base was indeed observed when the platinum was present in excess over AMPS (Strothkamp and Lippard, 1976). Further evidence of high affinity of a sulfur towards platinum came from the base pre-binding experiments: in which [(terpy)Pt(NO₃)]⁺ was allowed to react with base (presumably through N7/N1 atoms of adenosine) in the presence of 150-fold excess of both AMP and UMP for 45 min at 25° C. However, addition of 1 mole of UMPS per mole of platinum into this mixture resulted in the quantitative formation of [(terpy)Pt(UMPS)] within a few minutes suggesting that rearrangement from Pt-base binding into Pt—S binding occurred (Strothkamp and Lippard, 1976).

The selectivity of [(terpy)ptX]ⁿ⁺ towards compact phosphorothioate moieties over the bulky base is enhanced by the large terpyridine ligand coordinated to Pt²⁺. A sterically hindered system around platinum should result in a sharp decrease in reaction rate (Howe-Grant and Lippard, 1980) between [(terpy)Pt] and a base, especially in structured polynucleotides having base stacking interactions, involvement in H-bonding, or tertiary structure cages. Also, the strong intercalative binding of terpyridineplatinum(II) complexes to double helix decrease their reactivity towards bases participating in the stacking interaction (Lippard, 1980). The acidotriaminoplatinum structure of [(dien)PtCl]Cl (FIG. 15) and, therefore, its chemical properties as a monodentate reagent (with only one reactive coordinate, Pt—Cl) are similar to [(terpy)PtCl]Cl (FIG. 15). However, the diethylenetriamine ligand is much more compact than terpyridine and has no intercalation ability. Slavin et al. (1994) established that [(dien)Pt]²⁺ also exclusively binds to sulfur in adenosine phosphorothioates (AMPS, ADP-β-S, and ATP-γ-S), over the temperature range of 25 to 40 úC even though the purine nitrogens (N7 and N1) are available for coordination at pH 6.5. The complexes are formed mainly through the second order reaction with phosphorothioate nucleotides):
[(dien)PtCl]++AMPS->[(dien)Pt(Nu)].
   In contrast, the platination of unmodified GMP (through N7) and AMP (through N7 and N1) by the same platinum reagent has a mixed mechanism proceeding through both direct reaction (the minor pathway) and indirect reaction through formation of more reactive aquoplatinum complex before interaction with nucleotides (the major pathway):
[(dien)PtCl]++H₂O->[(dien)Pt(H₂O)]²⁺
[(dien)Pt(H₂O)]²⁺+NMP->[(dien)Pt(NMP)]
   The magnitude of the second-order rate constant for thionucleotides is about 20-fold greater than that for GMP and about 50-fold greater than for AMP (Slavin et al. 1994).

Since sulfur reacts with the [Pt-amine] compounds mainly through the directly substitution of the Cl⁻ ligand without prior aquotation, the reaction between [(dien)PtCl]⁺ and glutathione (HSDR) is nearly independent of the [Cl⁻] concentration whereas the reaction with GMP can be completely inhibited at high concentration of NaCl (Reedijk, 1991). In contrast, [(dien)Pt(H₂O)]²⁺ will almost selectively react with GMP (Reedijk, 1991). Elmroth and Lippard (1994) showed that the rate of platination of GG site (through N7) in d(TTTTTTTTGGTTTTTTT) by cis-[Pt(NH₃)(NH₂C₆H₁₁)Cl(H₂O)]⁺ is approximately 35-fold higher than that in the dinucleotide d(GG), and only about 3-fold less reactive than the phosphorothioate site in d(TTTTTTT_{S}TTTTTTT), irrespective of whether the oligonucleotide was single- or double-stranded oligonucleotide structure.

In the reaction with platinum reagents, Gₙ clusters (where n³2) are the most reactive sites in DNA (Bruhn et al., 1990; Lepre and Lippard, 1990; Gonnet et al., 1996). At GGG (and longer G tracts), the N7 of the central residue is the most nucleophilic site(s), and [(dien)PtCl]⁺ preferentially attacks this site (Yohannes et al., 1993).

Theoretically, if one [L₃Pt]²⁺ group (where L is an amino ligand) is already attached to a guanine, it is expected to repel a second one reacting with an adjacent guanine. However in model experiments using [(NH₃)₃PtCl]⁺ (FIG. 15), such repulsive effect of the first [(NH₃)₃Pt]²⁺ group tethered to a guanine was found to be very moderate and did not prevent the platination of the second guanine in d(CTGGCTCA) even under stoicheometric conditions (Reeder et al., 1996). This result opens the possibility of a multiple platination of adjacent guanines in Gₙ clusters.

Therefore, in contrast to homopyrimidine phosphorothioate oligonucleotides, where selective platination of POS residues eventually can be obtained, the platination of the oligonucleotides containing both phosphorothioate group(s) and Gₙ clusters will result in a mixture of P—S and Pt-N7(Gua) adducts.

Further details of the invention are illustrated in the following non-limiting examples.

### EXAMPLE 1

### Antisense-Mediated Down-Regulation of Tumor Necrosis Factor Alpha (TNFα)

Tumor necrosis factor alpha (TNFα) plays an important role in the immune response to infection. However, exaggerated production of this cytokine (also called cachetin) can lead to cytotoxicity, organ failure and death in the case of septic shock (see, e.g., Beutler and Grau, Crit. Care Med. 21:S423-435 (1993) and Dinarello, J. Infect. Dis. 163:1177-1184 (1991)). Moreover, TNFα, along with interleukin-1, has been shown to mediate the pathogenesis of chronic inflammatory joint diseases such as arthritis (Probert et al., Eur. J. Immunol. 25:1794-1797 (1995)) as well as cachexia, or wasting syndrome (Tracey and Cerami, Ann. N.Y. Acad. Sci. 569:211-218 (1989)). Antibodies directed against TNFα have been shown to protect against the lethal effects of septic shock and cachexia (Beutler and Grau (1993), supra and Tracey and Cerami (1989), supra), indicating that TNFα is a good candidate for antisense and antigene therapy.

As presented below, we have tested the effectiveness of the above described method for down-regulating TNFα expression in macrophages both in vitro and in vivo. Specifically, we have tested the ability of preformed antisense-TNFα mRNA complexes to block ribosome passage during translation in vitro and have compared our current constructs with the antisense sequence alone. We have also tested the effectiveness of our constructs for inhibiting TNFα production in macrophage-like cell lines and macrophages in mice using delivery procedures already proven for this system.

A. Construction and Structure of Antisense Molecules

Chimeric RNAs consisting of the minimal hairpin ribozyme sequence plus antisense and triplex-forming moieties (ATR 1) targeted to the intended target sequence of TNFα mRNA were designed as shown schematically in FIG. 2A. A 150-bp DNA fragment encoding the T7 promotor, a 21-nt sequence complementary to a pre-selected region of TNFα RNA (“A”), a potential triplex-forming sequence (“T”), and the sequence of the minimal hairpin ribozyme was assembled from four overlapping oligonucleotides using T4 DNA ligase, amplified by the polymerase chain reaction (PCR), and transcribed by T7 RNA polymerase to generate the precursor (pre-ATR 1) RNA. Control experiments used an RNA species designated “AT” which possessed the antisense and triplex forming sequences required for forming a complex with the TNFα RNA target but which lacks the catalytic hairpin ribozyme domain.

Self-processing at 37° C. of primary pre-ATR 1 RNA during transcription at the sites flanking the mature ATR 1 sequence (see “cleavage/ligation” sites in FIG. 2A) resulted in the production of a number of RNA species (designated R2, R3a, R3b and R4) (FIG. 2E). By comparison of the length and analyzing the end structures of these individual gel-purified RNA species and the ways by which they can interconvert, we have unequivocally identified them as unprocessed transcript (R2), semi-processed linear ATR 1 (R3a and R3b), fully processed linear ATR 1 RNA (R4), and the circular form of mature ATR 1 RNA (not shown). All these RNA processing events are the result of autocatalytic cleavage by the internal ribozyme moiety. The identification of these RNA species was supported by 5′- and 3′-end labeling experiments (data nor shown). We have found that the interconversion of the linear R4 and circular species by self-ligation and self-cleavage (FIG. 2F) occurs under a wide range of conditions, which is in agreement with data obtained for other hairpin “miniribozymes” (Buzayan et al. (1986), supra, Hampel and Tritz (1989), supra, Chowrira et al., Biochemistry 32:1088-1095 (1993) and Feldstein and Bruening (1993), supra).

FIGS. 2B and 2C show the putative secondary structures of the complexes formed between the TNFα RNA target and the ATR 1 and AT antisense RNA species. Specifically, in the design of the ATR 1 structure, we incorporated a triplex-forming element in order to bring the ends of the hairpin ribozyme domain into proximity so as to favor ligation into the covalently closed circular form (see FIG. 2B). After initial binding of the antisense sequence to the TNFα mRNA, covalent closure of the hairpin ribozyme domain may be facilitated by formation of the triple helix with the other end of the n-loop, which brings the P5 domain of the minimonomer near to the D8 end. The lengths of the duplex and triplex regions were chosen so that the two molecules will be intertwined and thus unable to separate.

The linear species (R4) can fully pair with the target upon binding, as long as the folded structure of the ribozyme can open. After pairing, the ends are again able to approach each other, perhaps aided by formation of a triplex region, as shown in FIG. 2B. Conditions allowing refolding of the ribozyme into its native conformation is necessary for strong binding, which should be maximized by creating a linkage of the ends around the target. A new spontaneous ligation event would result in covalent linkage of the antisense and target RNAs. Because the two ends are now held in proximity by the triplex as well as the P5 helix, the ligation rate should increase while the cleavage rate remains about the same, therefore the equilibrium should shift to the ligated, covalently linked state. The mRNA can be freed from this structure only by spontaneous cleavage followed by unwinding of the triplex and then unwinding of the duplex. The likelihood of all three of these events occurring is expected to be very small.

B. Improved Binding Characteristics of ATR 1 Antisense Oligonucleotides

An RNA molecule containing the first 709 nt of the TNFα mRNA (designated herein as “TNF1”) was transcribed using the pGEM-4 vector system and T7 RNA polymerase and was employed as a target for various antisense RNA molecules. Specifically, an autoradiogram was made after electrophoresis on 6% denaturing (8 M urea, 2 mM EDTA) polyacrylamide gel of the gel-purified linear ATR 1 RNA (“R4” form) in equilibrium with its circular “R1” form (lanes 1-6 of FIG. 3) and AT RNA (lanes 7-12 of FIG. 3), all internally labeled by [α³²P]CTP with T7 RNA polymerase, after incubation alone (lanes 1, 4, 7 and 10 of FIG. 3), or with either 0.1 μg/μl TNF1 RNA (lanes 2, 5, 8 and 11 of FIG. 3) or 0.2 μg/μl TNF1 (lanes 3, 6, 9, and 12 of FIG. 3) in 50 mM Tris-HCl (pH 8.0), 10 mM MgCl₂ for 60 min. at 37° C. All samples were mixed with equal volumes of 2×FLS (standard gel loading solution containing 90% formamide and 10 mM EDTA) and incubated either for 5 min. at 37° C. (lanes 1-6 of FIG. 3) or for 2 min. at 95° C. (lanes 7-12 of FIG. 3) before electrophoresis.

We found that ATR 1 antisense RNAs could form ultrastrong complexes with the target TNFα RNA molecule. These complexes appeared to be stable enough to be detected as several individual bands with close mobility when examined by denaturing electrophoresis in 6-8% denaturing polyacrylamide gels containing 8M urea, 2 mM EDTA at 45° C. (see FIG. 3, lanes 2-3). These findings agree with data reported on retardation of highly structured RNAs in denaturing polyacrylamide gels (Dante et al., Anal. Biochem. 225:348-351 (1995)). Moreover, the electrophoretic mobility of complexes between the [³²P]-labeled chimeric RNAs and nonradioactive TNF1 RNA was slightly retarded compared to that of [³²P]-labeled TNF1 alone.

In control experiments we used the AT RNA which, as described above, lacks the hairpin ribozyme domain but retains the sequences capable of forming the specific complex(es) with the TNF1 RNA (see FIG. 2C), as well as “m101”, a control RNA containing the minimal hairpin ribozyme domain plus an irrelevant sequence in place of the antisense and triplex forming sequences (see FIG. 2D). Complexes formed between [³²P]-labeled AT RNA and non-radioactive TNF1 under the optimum conditions dissociate during electrophoresis, producing a smear behind the principal band of AT RNA (FIG. 3, lanes 5-6). Similar smearing has been reported for the gel-electrophoresis analysis of complexes formed between the hairpin ribozyme and its substrate analogs that are additionally stabilized by a long intermolecular duplex (Feldstein et al., Proc. Natl. Acad. Sci. USA 87:2623-2627 (1990)). No specific retardation or smearing was detected during similar gel-electrophoretic analysis of mixtures of [³²P]-labeled m101 and 0.02-0.2 μg/μl TNF1 RNAs (data not shown).

When the RNA complexes were dissolved in 45% formamide and 5 mM EDTA, heated at 95° C. for 2 min. and subjected to polyacrylamide gel electrophoresis under denaturing conditions, only a small amount of the ultrastrong complexes between TNF1 and [³²P]-labeled ATR 1 RNA was detected, and all less stable complexes inducing the smearing were abolished under these conditions (FIG. 3, lanes 7-12). We found that the high temperature extraction of M²⁺ ions from this RNA complex by excess EDTA was the key factor resulting in irreversible dissociation of these complexes. Moreover, the ultrastrong complex between the TNF1 RNA and [³²P]-labeled R1/R4 ATR 1 RNAs could not be formed when magnesium ions were replaced by either Na-EDTA or manganese ions (data not shown), consistent with the fact that Mg²⁺ supports HPR folding into the active conformation whereas Mn²⁺ does not.

Kinetic and gel-shift analysis of the formation of complexes between TNF1 RNA and the antisense RNAs showed that initial binding (presumably through ordinary Watson-Crick sense-antisense base pairing) is rapid, relatively weak, and occurs with roughly the same binding constants and rates of formation for both AT and ATR 1 RNAs (data not shown). These primary complexes can be detected by gel electrophoresis under nondenaturing (but not denaturing) conditions.

C. Assessment of the Ability of ATR Antisense RNAs To Block Ribosome Scanning or Translation

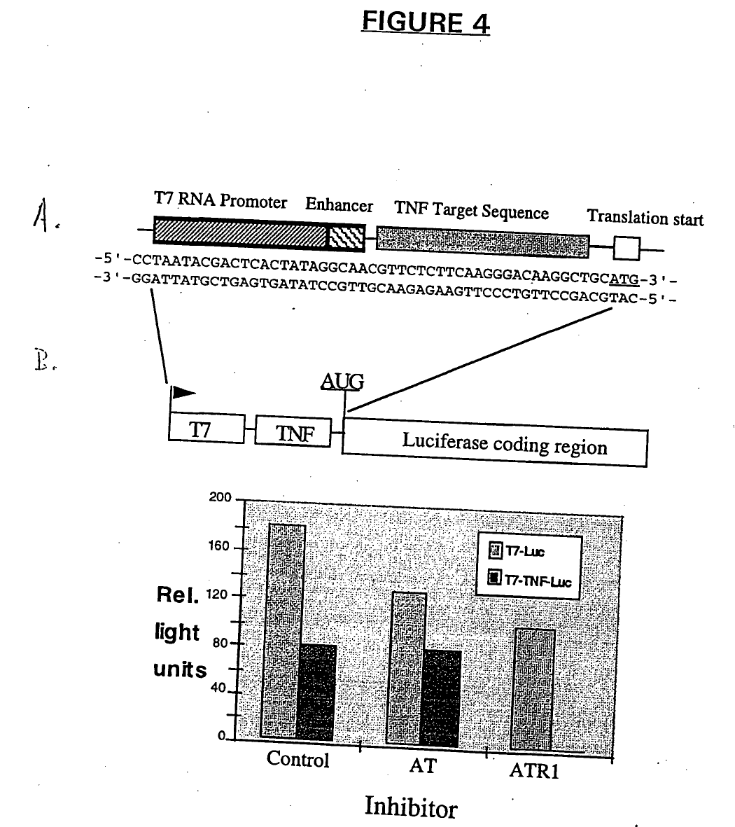

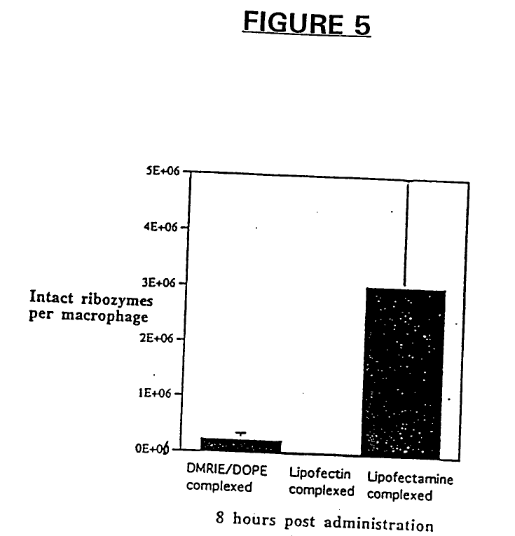

Since we have demonstrated greatly enhanced stability of binding between ATR 1 antisense RNA and a TNFα RNA target, we next tested how well this strong complex is able to block ribosome scanning of the 5′ untranslated region of an mRNA template or translation from that template. We, therefore, constructed a TNFα-luciferase fusion (designated herein as “PTS” for “Promoter-Target-Start Codon”) by inserting a TNFα target sequence flanked by a T7 promoter transcription enhancer and an AUG translation start codon into the NcoI site of pGL3 Control Vector (Promega, Madison, Wis.), a luciferase vector containing an SV40 translation promoter and enhancer (see FIG. 4A). By partly filling in the opened NcoI site before ligating with the insert, we eliminated the normal AUG and introduced a new AUG just after the TNFα sequence. This plasmid construct was linearized at an appropriate site, transcribed, and the transcripts were capped using the Ribomax in vitro transcription kit (Promega, Madison, Wis.).

The PTS fusion was pre-incubated in 50 mM Tris acetate (pH 7.5)/10 mM magnesium acetate, either alone or in the presence of a 10-to-40-fold molar excess of either ATR 1 or AT antisense RNA for 1 hr at 37° C. The resulting sense-antisense complexes were then added to a rabbit reticulocyte lysate (Promega, Madison, Wis.). Following in vitro translation of the complexes, luciferase production was measured. A T7 promoter-luciferase expression plasmid (designated herein as “PS”) lacking the TNFα target sequence was used as a further control.

The results of these experiments are presented in FIG. 4B. Specifically, the results in FIG. 4B demonstrate that ATR 1 antisense RNA is much more effective at inhibiting translation of PTS mRNA than is the AT antisense RNA. For an optimal [ATR1:target] molar ratio of 30:1, incubation of ATR 1 antisense RNA with PTS mRNA caused ≧95% inhibition of its translation, whereas hybridization with PS lacking the TNFα sequence resulted in almost no suppression of translation. We assume that the slight sequence non-specific inhibition of control PS mRNA is due to the chance occurrence of short regions of complementarity between these RNAs that could lead to the formation of complexes under the conditions of the translation reaction. Such observations are common for the RNA-RNA antisense approach (Eguchi et al., Ann. Rev. Biochem. 60:631-652 (1991)) and could be reduced by intracellular “proof-reading” protein cofactors.

D. Other Anti-TNF ATRs

The ATR 16a, ATR 16b and ALR 229 constructs (see FIG. 7) are similar to the ATR 1 construct except that they are directed to different regions of the TNFα target molecule. ATR 16a differs from ATR 1 in that is is targeted to a different homopurine sequence, which is located in the 5′ UTR of the TNF RNA, it has a shorter triplex-forming sequence (to ensure that there would be more turns of duplex rather than triplex in the complex) and the triplex forming sequence was proximal to the ribozyme ligation site, wherein in ATR 1 it is distal. ATR 16b is identical to ATR 16a except that the linker connecting the triplex-forming region and the helix adjacent to the ligation site is longer. ALR 229 contains no triplex-forming region, but instead an (AAC)₆ loop (a sequence chosen to provide some self-stacking but no self-pairing structure). Hence, ALR 229 is not restricted to targeting triplex-forming homopurine sequences but, like ATR 1, is targeted to a coding region. Each of our four ATR/ALRs contained a sequence feature intended to shift the cleavage-ligation equilibrium more toward ligation upon target binding to maximize chances of formation of stably linked structures. For ATR 1, ATR 16a and ATR 16b, formation of the triplex upon binding was intended to help stabilize the folded structure. For ALR 229, which lacks a triplex-forming sequence, we employed a different approach. Specifically, we changed the sequence adjacent to the ligation site to 5′-UCAGCC-3′ so that it would be complementary to a 5′-GGCUGA-3′ block within the antisense sequence. This change normally would not affect catalytic activity, however, pairing of these two sequences destabilized the normal ribozyme folding. As a result, when transcribed from its DNA template, ALR 229 undergoes only partial processing and does not generate either the mature linear or the circle in the absence of the target. Hence, it remains as a partially processed linear molecule able to wrap around the target without steric hindrance. Upon addition to the target RNA, processing and circle formation proceed efficiently, due presumably to disruption of this internal base pairing by the target RNA, allowing the rest of the ribozyme to fold into the catalytically active conformation and permitting ligation to occur. Although it was not guaranteed that subsequent recleavage would not also occur, the overall effect was expected to favor covalent linkage due to Le Chatelier's principle of mass action.

The ability of the new ATR 16a, ATR 16b and ALR 229 constructs to form strong complexes with TNFα RNA is shown in FIG. 11. Specifically, FIG. 11 shows gel-shift analyses on denaturing gels. The results in FIG. 11 demonstrate that each of the ATR and ALR constructs tested are capable of forming strong complexes with target TNFα RNA. The radiolabeled target RNA (“TT”) did not contain the binding site for ATR1, which was used as a negative control in this experiment and, as expected, did not show any retarded complex.

E. Kinetics of Strong Complex Formation

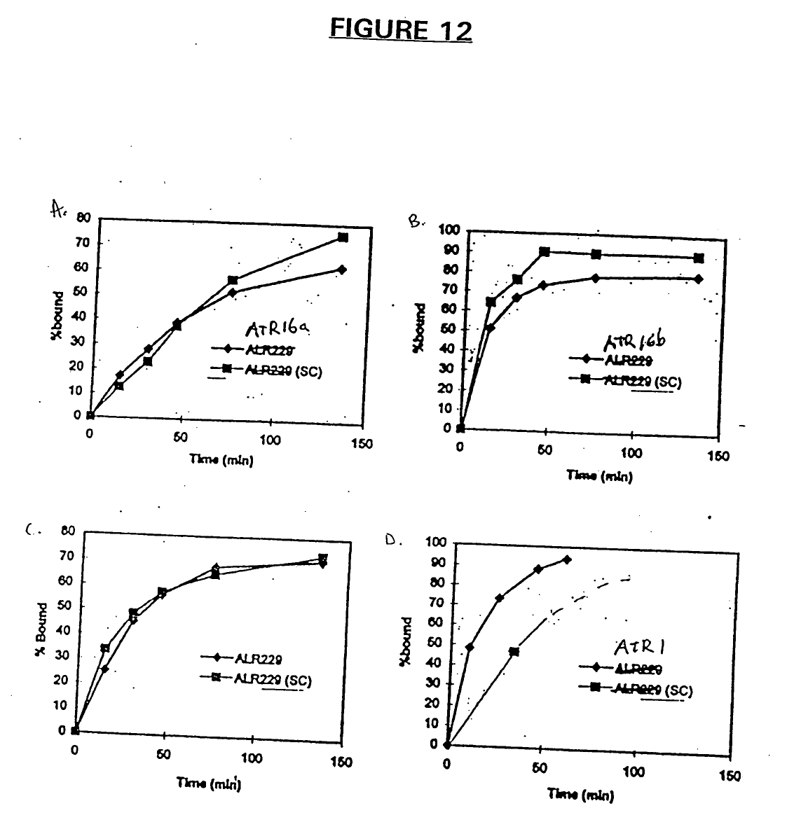

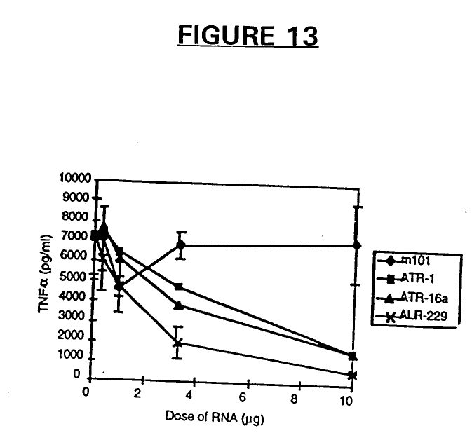

To understand relative rates of hybridization and strong complex formation, we removed aliquots of ATR-target complexes after different times of incubation together and electrophoresed one-half of each aliquot on a nondenaturing gel (to detect hybridized complexes, either strong or weak) and the other half on a denaturing gel. For ATR 16a, ATR 16b and ALR 229, the inital kinetics were almost identical for both complementary hybridization and strong complex formation (FIGS. 12A-C), showing that hybridization is the rate-limiting step. In contrast, the analysis of binding ATR 1 with appropriate TNF target (FIG. 12D) showed that initial binding through hybridization of sense and antisense sequences is relatively rapid and occurs with roughly the same binding constants and rates of formation for both AT and ATR 1 RNAs (t_{1/2}=10 minutes at 37° C., 10 mM MgCl₂ and 50 mM Tris-HCl, pH=8.0). The formation of strong complexes by ATR 1 RNA is a slower process (t_{1/2}=40 min.) than the initial binding. Moreover, radiolabeled ATR 1 RNA can be displaced from the initial complex with TNF1 RNA by the addition of an excess of unlabeled AT RNA, but no such substitution is detected after formation of the strong complex. Rapid strong complex formation in case ATR 16a, ATR 16b and ALR 229 makes these constructs superior over ATR 1.

F. Delivery of ATR 1 Antisense Constructs to Macrophages for Down-Regulating TNFα

Effective delivery of biologically active RNA (Malone et al., Proc. Natl. Acad. Sci. USA 86:6077-6081 (1989)) and ribozymes (Sioud et al., J. Mol. Biol. 223:831-0835 (1992)) has been demonstrated for cells of hematopoietic origin. Lipofectamine (DMRIE/DOPE; Life Technologies, Bethesda, Md.) has been used to introduce our constructs into murine peritoneal macrophages both in vivo and in vitro because previous studies had shown it to be superior to a variety of other lipid formulations, with low toxicity (Kisich and Erickson, J. Leukocyte Biol. Suppl. 2:70 (abstract) (1991a) and Kisich and Erickson, FASEB J. 4:1860 (abstract) (1991b)).

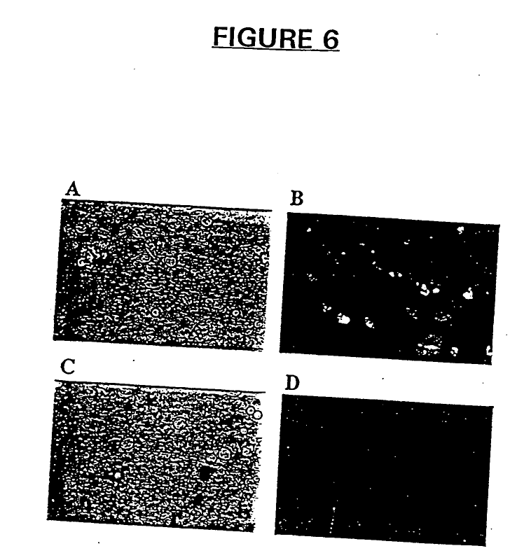

For example, 10 μg of ³²P-labeled hairpin ribozyme (HPR) was complexed with either Lipofectin (DOTMA/DOPE), DMRIE/DOPE, or Lipofectamine and administered i.p. to mice in a volume of 1 ml. Macrophages were harvested after 8 hours and lysed in 97% formamide 5 mM EDTA, 0.1% SDS. The lysates were analyzed by gel electrophoresis and phosphor-imaging to calculate the number of intact HPR molecules per macrophage. The results are presented in FIG. 5.

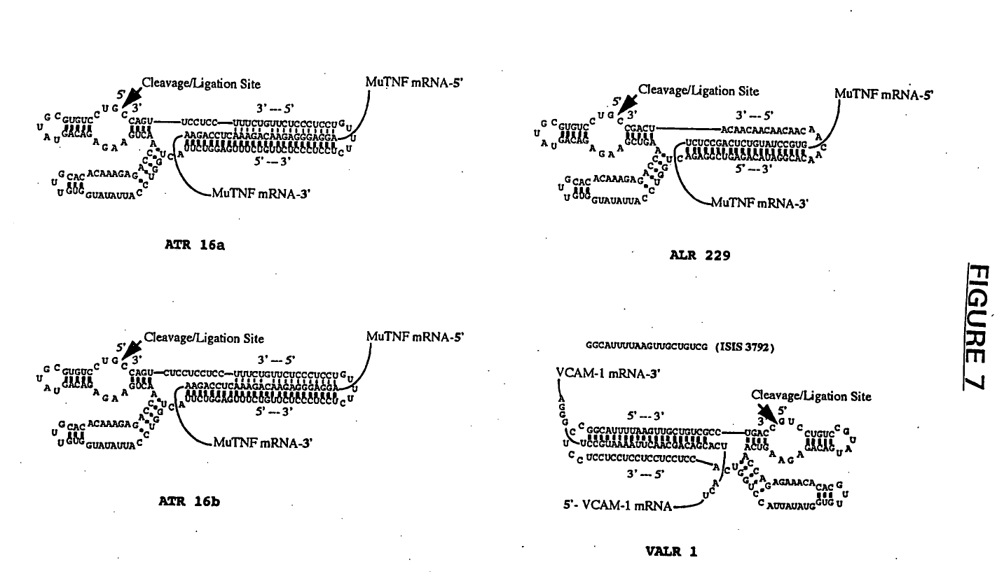

As shown in FIG. 5, primary macrophages isolated from mice that have been given a single intraperitoneal administration of 10 μg of HPR/Lipofectamine complex accumulate approximately 3×10⁶ molecules per cell, far more than with DMRIE/DOPE or Lipofectin. The molecules taken up persist for at least 24 hours (data not shown). Greater than 90% of the macrophages harvested after a similar delivery of fluorescein-conjugated HPR were fluorescein-positive. Moreover, the cellular distribution of the HPR was both nuclear and cytoplasmic (see FIGS. 6A and B). Delivery of constructs in this manner is fairly specific for macrophages, the primary source of TNFα, as lymphocytes have no detectable fluorescence. HPR administered without Lipofectamine accumulates poorly in peritoneal macrophages (FIGS. 6C and D). Based on these data, the use of Lipofectamine to deliver our constructs to macrophages both in vivo and in vitro was judged to be adequate for the desired effect.

To produce responsive macrophages, 1 ml of aged sterile fluid thioglycollate broth (Difco, Detroit, Mich.) was injected i.p. into 6-week-old female C57b1/6NCR mice 3 days before peritoneal lavage. The resulting peritoneal exudate cells (PEC) will be obtained by lavage using Hanks balanced salt solution (HBSS), plated at 1×10⁶/well in 24-well plates with Eagle's minimal essential medium (EMEM) with 10% heat-inactivated fetal bovine serum. After adhering for 2 hr, the wells were washed to remove nonadherent cells.

For in vitro transfection of macrophages or RAW264.7 cells (a macrophage-like cell line), Lipofectamine was diluted 1:4 with HBSS in polystyrene tubes, vortexed, and ribozyme construct were added at a 2.2-3.3:1 [DOSPA:RNA] phosphate charge ratio (Lipofectamine consists of DOSPA and DOPE in the w/w ratio of 3:1). After vortexing again, an amount of the mixture containing 1 μg of RNA were then added immediately to serum-free EMEM-rinsed cell cultures and allowed to incubate for 3 hr.

For in vivo transfection, the transfection reagents were prepared as described above except that the amounts were scaled up so that 10 μg RNA is used per mouse. After vortexing, 1 ml of the resulting [liposome:RNA] complexes were loaded into 1-cc syringes fitted with 30-gauge needles and then injected i.p. into mice that has previously been treated with thioglycollate to recruit responsive macrophages. Macrophages were harvested 3 hr later by peritoneal lavage with HBSS. The exudates were plated at 1×10⁶/well in 24-well plates and allowed to adhere for 2 hr, then washed with HBSS to remove nonadherent cells.

After transfection, macrophages isolated as described above, or RAW264.7 cells were stimulated with 100 ng/ml lipopolysaccharide (LPS) to induce TNFα production. Supernatants were sampled at 0, 2, 4, 8, and 24 hr post-LPS stimulation and stored at −70° C. until quantitation, which were done by a TNFα-specific ELISA (Biosource International).

G. Development of Vectors For Endogenous Delivery of ATR 1 Antisense RNAs

To demonstrate the feasibility of using ATR antisense RNAs in a gene therapy setting, we have incorporated the genes encoding these molecules into eukaryotic expression vectors. The strategy for construction of the mammalian high expression DNA vectors for ATR generation is as follows. Single copy or concatomer copies of the ATR DNA templates are generated by PCR reactions and cloned into the CMV promoter driven pS65T-GFP-C1 vector (Clontech Labs, Palo Alto, Calif.) downstream from the GFP (green fluorescent protein) expressing region. The T7 promoted vector used for our ATR in vitro synthesis was used as DNA template for generation of mammalian cloning sequences. Using a 5′ overhang primer, restriction cloning sites for EcoRI and BglII and a eukaryotic stop codon are introduced upstream of the ATR sequence, eliminating the T7 promoter. Single ATR copy expressing vectors are constructed by cloning of the ATR template into the EcoRI-BamHI site in the MCS of the GFP vector. Concatomer copy expressing vectors are generated by creating a head-to-tail multimeric ligation between compatible cohesive ends of BglII head and BamHI tail sites. Head-to-head and tail to tail ligations are inhibited by having BglII and BamHI enzymes present in the ligation mixture (at 100 mM NaCl to avoid “star” activity). Head-to-tail ligations will not be cleaved by these enzymes. The final ladder product is isolated from acrylamide gel electrophoresis and cloned into the BglII-BamHI site of the GFP vector. In-frame directional clones are selected by the characteristic of being cleaved by these two enzymes.

The expression constructs will be introduced into the murine macrophage-like cell line, RAW264.7, via electroporation (at 960 μFd and 230 V, which has been previously shown to be optimal for this cell line) or lipofection with Lipofectin complexes with the DNA at a 2:1 charge ratio. Expression levels of the ATR antisense RNAs will be assessed by Northern analysis or RT-PCR (Sambrook et al. (1989), supra). Efficacy of the plasmid expressed triple helix antisense ribozymes will be assessed following stimulation of the transfected cells with lipopolysaccharide, followed by ELISA for secreted TNFα in the supernatants. Constructs that prove effective in this transient expression assay may then be incorporated into adenoviral or adeno-associated viral vectors (Kozarsky and Wilson, Curr. Op. Gen. Dev. 3:499-503 (1993) and Xiao et al., Adv. Drug Del. Rev. 12:201-205 (1993)) for assessment of in vivo efficacy. Further optimization of the basic ATR cassette (ATR 1 without the antisense and triplex-forming sequences), if required, will be carried out by deletion analysis and isolation of improved variants from partially randomized sequence libraries. Finally, antigene applications, in which double-stranded DNA is targeted, will also be tested.

H. Conclusion

Our data revealing the superior stability of the padlock RNA-target complex suggest that padlock antisense RNAs can wind around the target RNA strand and topologically link themselves to the target RNA molecules through catenation, in a manner similar to a real padlock. The “lock” is evidently the hairpin ribozyme domain, since AT RNA cannot form such a strong complex with the TNF1 target. Circularized probe molecules should be restricted in one-dimensional diffusion along the target RNA in nondenaturing conditions because of the TNF1 target's secondary and tertiary structure. However, Nilsson et al. (1994), supra, showed that circular oligonucleotide probes are free to travel considerable distances along the target strand during denaturation. In agreement with these findings, we found that the ATR 1 antisense RNAs complexed with the 396-nt longer (in comparison with the 3′ end of TNF1) target “TNF2” RNA were more resistant to denaturing conditions than complexes with the shorter TNF1 RNA target (data not shown) (the sense sequence was 563 nt from the 5′ ends of both target RNAs).

We found that covalent circularization (self-ligation) of the hairpin ribozyme domain in ATR 1 antisense RNA is not strictly required for formation of the strong complex with the TNF1 target. Thus, as discussed above, we showed that linear ATR 1 RNA molecules having 5′-[³²P]-phosphate and 3′-OH ends (i.e, the “R4” form) failed to form the covalent circle R1 RNA. However, this “termini misphosphorylated” RNA molecule also could form strong complexes with the TNF1 target despite the fact that covalent cleavage should obviously result in the strongest padlock. More recent results suggest that a fraction of ligation-competent ribozyme-target complexes are significantly more stable, suggesting that these are covalently linked.

Since the structures of the hairpin ribozyme required for both self-cleavage and self-ligation were shown to be essentially identical (Chowrira et al. (1993), supra and Butcher and Burke (1994), supra) the covalent bond itself at the cleavage/ligation site presumably does not play a role in stabilization of the catalytically active ribozyme structure. A similar feature has been shown for circularly permuted tRNAs, which can form the same structure even when their nucleotide chains are interrupted by placing nicks in the middle of helices or in anticodon loops (Pan et al., Science 254:1361-1364 (1991)). Coaxial stacking across the cleavage sites is probably a factor in stabilizing these structures (Walter et al., Proc. Natl. Acad. Sci. USA 91:9218-9222 (1994)). Recent results (Butcher and Burke (1994), supra) indicate that the hairpin ribozyme adopts a stable, magnesium-dependent tertiary structure where the sequences adjacent to the cleavage/ligation site are likely involved in non-Watson-Crick base pairing interactions.

### EXAMPLE II

### Inhibition of TNFα Secretion by ATR Constructs

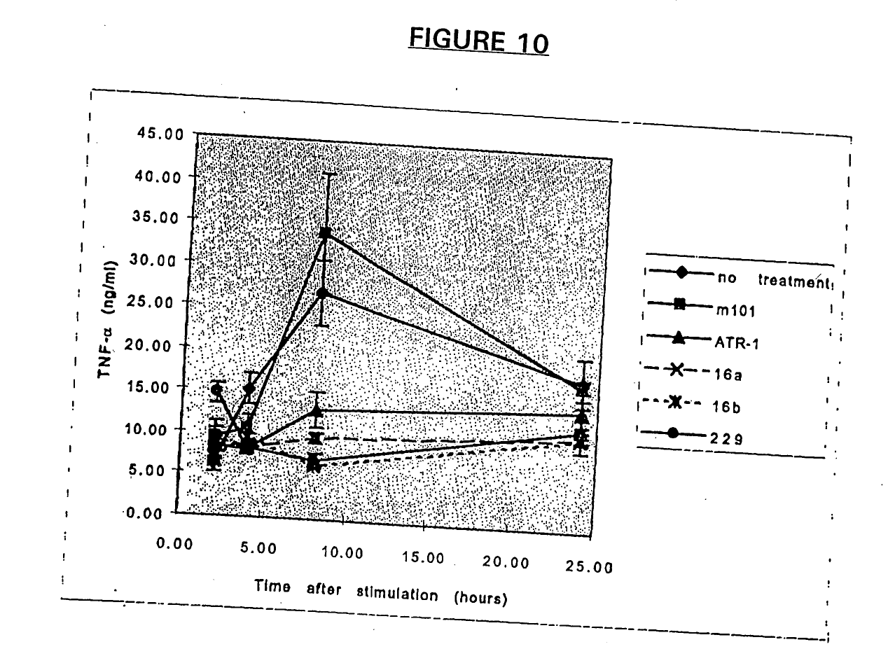

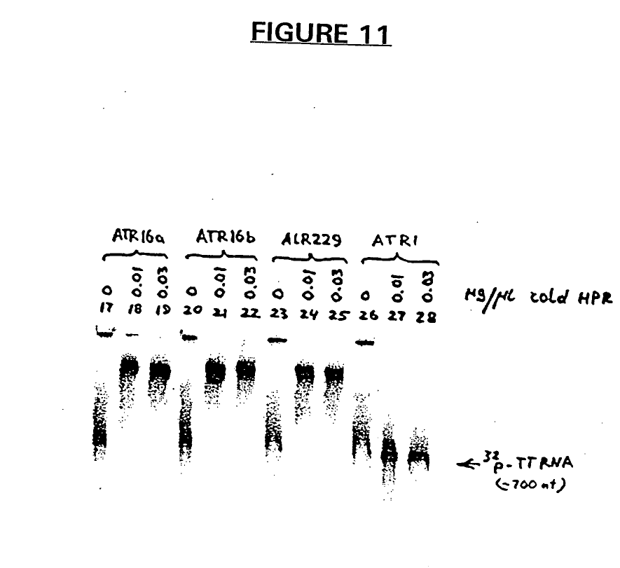

The ability of various anti-TNFα antisense constructs to inhibit the secretion of TNFα from RAW264.7 cells was determined. Specifically, 2×10⁵ RAW264.7 cells were treated with 4.5 μg of the antisense construct ATR 1, ATR 16a, ATR 16b or ALR 229 or control RNA (m101) as described in Section I-F above. The RNA was complexed with Lipofectamine at a 3:3:1 charge ratio for 2 hours in 1 ml DMEM. TNFα levels in supernatents were measured by specific ELISA at increasing intervals after stimulation with 100 ng/ml LPS. The results of these experiments are presented in FIG. 10.

As shown in FIG. 10, each of the padlock RNAs ATR 1, ATR 16a, ATR 16b and ALR 229, were able to significantly inhibit the secretion of the TNFα protein by RAW 264.7 cells grown in culture. In contrast, cells treated with control RNA (m101) or untreated cells still produced TNFα at significantly higher levels than the antisense treated cells. These results demonstrate that the constructs ATR 1, ATR 16a, ATR 16b and ALR 229 are capable of inhibiting the expression of the TNFα protein in cultured cells treated with these constructs.

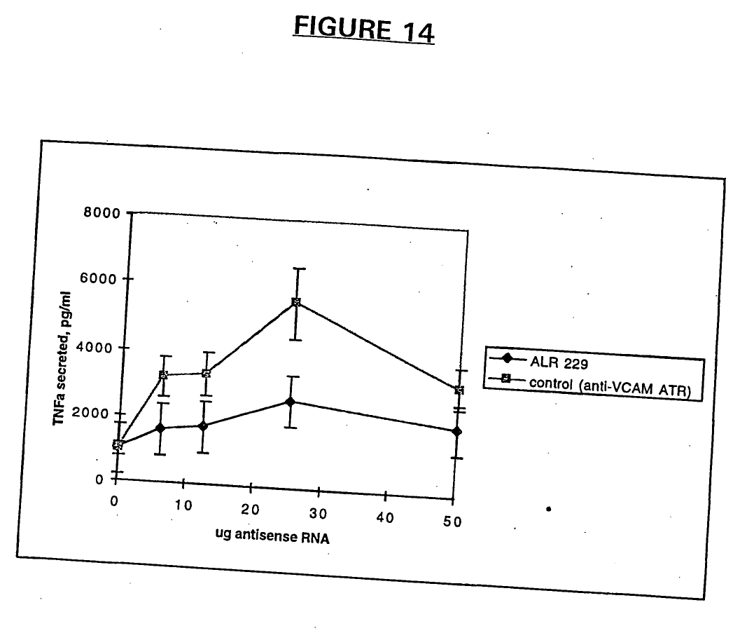

The above experiments were repeated identically except that the lipid:RNA charge ratio of the transfection complex was lowered from 3.3:1 to 2.2:1. This resulted in improved results. This improvement is evident in the dose response curves shown in FIG. 13, where TNFα secretion at the 8-h time point is plotted against RNA dose. Here ALR229 was revealed to be the most effective, inhibiting TNFα secretion by 90% at a level (10 μg per well of 2×10⁵ cells each) that caused no nonspecific toxicity, as indicated by the lack of fall-off in secretion with m101 at that level. ATR 16a turned out to be more effective than ATR 16b in this assay (not shown), demonstrating that the shorter linker provided apparently more favorable binding characteristics.

These data demonstrate the following points:

1. A Lipofectamine:RNA charge ratio of 2.2:1 is much less toxic than the 3.3:1 ratio previously used, for example in FIG. 10. This formulation permits more than double the dose used previously without reducing TNFα secretion in the control group (which received a construct (m101) lacking the antisense sequence).

2. Target selection is not limited to homopurine blocks but can be any sequence. For example, ALR 229, which contains no triplex-forming sequence and is targeted at a non-homopurine sequence, is the most potent antisense of these molecules, with an IC₅₀ of about 46 nM.

3. It is possible to efficiently target coding regions in cells using antisense molecules (ATR 1, ALR 229) that do not depend on cleavage of their target mRNA for their effectiveness (in contrast to hammerhead ribozymes, or antisense DNAs that rely on cleavage by RNase H). This has never before been achieved, to the best of our knowledge.

4. Our newer padlock RNA constructs are even better than our original ATR 1; hence the ATR approach can be further improved by design innovations. At the same time, the fact that three or four out of four constructs designed were effective in cell assays indicates that our basic design principles are quite general, rather than being limited to a few instances of accessible target sequences.

### EXAMPLE III

### Inhibition of TNFα in Mice

We chose our most effective padlock RNA in the cell culture assay to test for in vivo efficacy in mice (FIG. 14). As with the in vitro assays, ALR 229 was complexed with Lipofectamine and delivered i.p., after which macrophages were recovered and assayed in vitro for TNFα production following LPS stimulation as described in the brief description of FIG. 14. Although there was some nonspecific stimulation of TNFα secretion apparently due to the proinflammatory effects of Lipofectamine, mice that had received ALR 229 consistently showed half the level of TNFα production shown by mice that had received a control ATR that was directed at an irrelevant gene (human VCAM-1; the human VCAM-1 ATR is not expected to bind to the mouse VCAM gene or any other gene). We consider this strong evidence that ALR 229 has a significant antisense activity in vivo. It also suggests that Lipofectamine, and perhaps cationic lipids in general, may not be the best vehicle for in vivo delivery of anti-TNFα agents.

### EXAMPLE IV

### Antisense-Mediated Down-Regulation of VCAM

In response to injury or infection, leukocytes adhere to endothelial cells lining the walls of blood vessels in the area and proceed to emigrate through the wall and into the affected tissue. This process is mediated by the cytokine-induced expression of several adhesion molecules on the endothelial cell surface, including members of the selectin family (P-selectin, E-selectin) (Lawrence et al., Cell 65:859 (1991)) and members of the immunoglobulin family (ICAM-1, ICAM-2 and VCAM-1) (Oppenheimer-Marks et al., J. Immunol. 147:2913 (1991)). VCAM-1 is induced by IL-1, IL-4 and TNF, and reaches maximal levels 10-14 h after cytokine treatment, remaining elevated for up to 72 h (Rice and Bevilacqua, Science 246:1303 (1989) and Masinovsky et al., J. Immunol. 145:2886 (1990)).

The VCAM-1 gene, which is present in a single copy in the human genome, contains 9 exons spanning approximately 25 kilobases of DNA. Exons 2-8 contain C2 or H-type immunoglobulin domains. At least two different VCAM-1 precursors can be generated from the human gene as a result of alternative mRNA splicing events, which include or exclude exon 5 (Cybulsky et al., Proc. Natl. Acad. Sci. USA 88:7859 (1991)).

Sustained elevation of levels of VCAM-1 is associated with several pathologies, including atherosclerosis, retinosis, inflammatory bowel disease and asthma (Smith et al., Am. Rev. Respir. Dis. 148:S75-78 (1993), Gosset et al., Ann. N.Y. Acad. Sci. 725:163-172 (1994) and Ohkawara et al., Am. J. Respir. Cell. Mol. Biol. 12:4-12 (1995)). VCAM-1 is induced in rabbit aortic endothelium in vivo within 1 week after initiation of an atherogenic diet and is expressed in rabbit atherosclerotic lesions in vivo (Li et al., Am. J. Pathol. 143:1551-1559 (1993)). In hypercholesterolemic rabbits VCAM-1 may participate in initial monocyte recruitment to prelesional areas of arterial endothelium (Libby and Clinton, Nouv. Rev. Fr. Hematol. 34(supp):S47-53 (1992)). Therefore, the gene is an excellent candidate for antisense therapy as described herein.

The first step in applying this novel approach to the VCAM-1 gene was to synthesize DNA templates for an ALR antisense RNA (VALR1) targeted to a 20-nt site on VCAM-1 RNA, overlapping the AUG initiation codon (nt 636-655 of the genomic sequence [Cybulsky et al. (1991), supra]). This site was identical to the target site of a phosphorothioate oligodeoxyribonucleotide that was found to have significant activity in suppressing expression of VCAM-1 in HUVEC cells (Bennett et al., J. Immunol. 152:3530-3540 (1994)). This other oligonucleotide was “ISIS 3792”, directed against the AUG codon. When ISIS 3792 was synthesized as a 2′-O-methyl derivative, which does not support RNase H cleavage when hybridized to a target, antisense activity was lost, indicating that ISIS 3792 depends upon RNase H cleavage for its activity in cells (Bennett et al. (1994), supra). From these results we inferred that the site on VCAM mRNA complementary to ISIS 3792 was accessible to hybridization in vivo, and that it would be a good test of the ability of padlock RNA constructs to stall ribosome progression without depending on RNase H cleavage for their effectiveness. The antisense sequences of VALR1 and ISIS 3792 as well as the complementary sequence of VCAM mRNA (with start codon underlined) are as follows:

ISIS 3792
3′-GCTGTCGTTGAATTTTACGG-5′
VALR1 antisense sequence
3′-GCUGUCGUUGAAUUUUACGG-5′
VCAM-1 mRNA target sequence
5′-CGACAGCAACUUAAAAUGCCUGGGAAGA-3′

To the antisense sequence ribozyme and linker sequences were appended so that the expected structure of the resulting construct upon binding to its target would be as shown in FIG. 7. The VALR1-target complex contains approximately one full turn more of sense-antisense duplex than triplex. In this regard, if there is no excess duplex over triplex, there can be no linkage, as the turns of the third strand would unwind the turns of the duplex around the target.

Synthesis of the complete VALR1 ATR RNA, designated VALR1, was accomplished by T7 transcription of appropriate DNA templates as described above for the TNFα system. For a target site, we used VALRT 1, a 642-nt partial transcript of VCAM mRNA fused with a piece of pSP-luc+NF vector sequence. This RNA contained a 20-nt sequence around the AUG codon complementary to the antisense domain of the VALR 1 molecule (see figures), located 247 nucleotides (nt) downstream of the target 5′-end and 375 nt upstream of the 3′-end. VALR 1 was even more active than ATR 1 (TATR 1) in self-processing (self-cleavage) of its precursor RNAs but, in contrast to ATR 1, self-processed linear

VALR 1 RNA showed very little ability to circularize in the absence of a target. However, self-processing of the pre-VALR 1 RNAs in the presence VALRT 1 target resulted in formation of circular VALR 1 species, as detected by denaturing gel-electrophoresis. We also showed that VALR1 RNAs can form a strong complex with VALRT 1 RNA, stable after gel-electrophoresis in denaturing conditions (8 M urea, 2 mM EDTA, 45° C. for 2 hr.).

For testing the ability of VALR1 to inhibit VCAM-1 expression, control RNAs are synthesized that lack the catalytic hairpin ribozyme moiety or contain an inactive, mutated version of it, as well as ALRs that lack the target binding site. These controls allow an assessment of specificity of binding and the importance of strong binding and linkage for biological efficacy. Phosphodiester and phosphorothioate versions of ISIS 3792 are also used to permit head-to-head comparison of ALR constructs with the type of antisense molecules currently under development as pharmaceuticals.

VALR1 and other antisense constructs that show strong binding in cell-free assays, together with control RNAs, will be assayed for their ability to down-regulate VCAM-1 expression in human vascular endothelial cells (HUVEC) cells. If appropriate to understand HUVEC cell results, we will also perform in vitro translation of luciferase mRNAs that have been modified to carry the appropriate ALR target sequence in either the 5′ UTR or the coding region as described in Example I above. Inhibition of luciferase production in rabbit reticulocyte lysates from ALR target/luciferase fusion mRNAs which have been precomplexed with ALR RNA will indicate sufficient complex stability to inhibit translation. For testing blockage of translation inside the cell, we will transfect the above constructs as preformed plasmid-ALR complexes into HUVEC cells and monitor luciferase activity as with the cell-free procedure, comparing with activity using control RNAs or no antisense. In this case the fusion gene will be expressed from the SV40 promoter. In vivo testing will also be performed as described above for TNFα antisense molecules.

### EXAMPLE V

### Cell-Specific Antisense Therapeutics

Because cancer cells are in many respects like their normal counterparts, virtually all potent anticancer drugs lack the requisite specificity toward cancer cells alone and are, therefore, highly toxic. As such, we herein describe a novel method for achieving cancer cell specificity by providing an therapeutic antisense agent with two independent levels of recognition. For the antisense agent to be active against a cell, recognition at both levels must occur. One level consists of Watson-Crick pairing between a target mRNA and the antisense sequence within the agent. The agent is designed so that stable duplex formation requires binding of an additional molecule that is abundant only in cancer cells. This cell-specific entity is the second level of recognition. The antisense target gene can be any gene whose overexpression is associated with tumor progression or metastasis, and whose down-regulation is expected to normalize growth control. Although many such proteins will find use in the present method, we will herein use the HER-2 gene as the antisense target. Repression of HER-2 in mouse tumors leads to suppression of tumor growth and longer survival of the mice; hence it is an attractive target for antisense therapy. The structure and sequence of the binary agent will be optimized through biochemical assays for tightness and specificity of binding, and if necessary through selection from randomized sequences. Then its effectiveness at blocking translation will be tested, first in an in vitro translation system and then in cultured tumor cell lines.

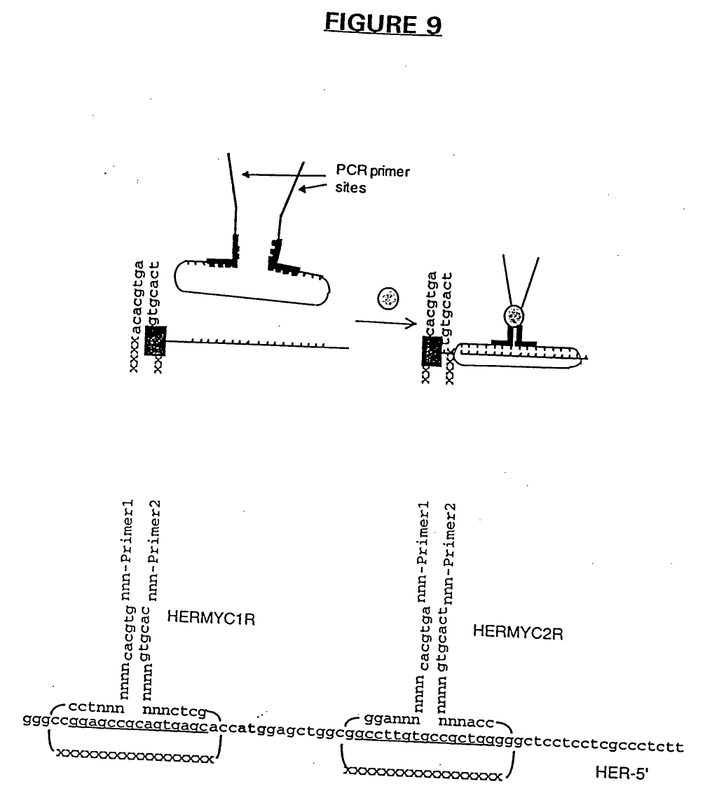

Clinical trials of antisense therapy for cancer are underway for at least two target genes relevant to cancer. However, a major limitation of this approach, and indeed of all drug therapy in current use for cancer, is the lack of ability to target the drug specifically to cancer cells. Thus, the goal of the presently described work is to employ a novel method for providing antisense therapy of breast cancer with this needed element of cell specificity. Treatment of breast cancer patients based on this approach will provide increased effectiveness and greatly decreased side effects. As described above, we have designed a ribozyme-based antisense agent intended to covalently link itself around its target mRNA after hybridizing to it. This linkage provided much greater strength of binding and potency in blocking translation. Here, however, we extend that approach to make the linkage dependent on the presence of c-myc, a nuclear protein present in elevated levels in breast cancer cells which binds to a specific DNA sequence. Thus, the antisense oligonucleotide binds to the mRNA target, wrapping around it due to the helical winding of the sense-antisense duplex. By bringing the ends closer together and reducing the conformational entropy of the molecule, this binding to the target permits the ends to pair with each other in an additional short, weak duplex which contains a binding site for c-myc (with its cofactor max) (see e.g., FIGS. 1C, D and H and FIG. 8 for a schematic illustration thereof). The binding energy of this sense-antisense complex by itself is too weak to suppress the target gene. However, if c-myc is present at elevated levels, it binds to the weak duplex, stabilizing it and “locking” the complex together by virtue of their helical interwinding. The antisense oligonucleotide can be either DNA or RNA, but DNA will be more stable against nuclease attack. The antisense oligonucleotide can bind to the target mRNA either by simple Watson-Crick pairing or by triplex formation. The scheme can be generalized to use theoretically any protein or other cell-specific molecule as the locking agent or “clasp,” and to target the mRNA of any desired gene. In the case of a clasp that does not normally bind sequence-specifically to a DNA or RNA, a two-part “aptomer” can be selected from randomized libraries of nucleic acid sequences that will have the appropriate binding properties, similar to the scheme shown in FIG. 9, see below). It can also be adapted to suppression of transcription by targeting the gene itself through triplex formation. Importantly, the clasp protein and the target gene need not be related, so two independent levels of selectivity for cancer are afforded.

The aggressively growing, invasive cancer cells which can become life-threatening in late-stage cancers are the end products of a series of genetic alterations that usually occur over many years. In the case of breast cancer, the genetic alterations that have been identified are mostly amplifications of a small number of oncogenes, among which are c-myc and HER-2 (c-erb-B2) (Van de Vijfer and Nusse, Biochim. Biophys. Acta 1072:33-50 (1991) and Kozbor and Croce, Cancer Res. 44:438-441 (1984)). Amplification of either of these genes is associated with aggressive breast cancer and poor prognosis (Wong et al., Am. J. Med. 92:539-548 (1992)). Transgenic mice containing the c-myc gene driven by strong promoters develop adenocarcinomas of the breast during pregnancy (Stewart et al., Cell 38:627-637 (1984) and Leder et al., Cell 45:485-495 (1986)). Similarly, overexpression of HER-2 has been shown to enhance malignancy and metastasis phenotypes (Hung et al., Gene 159:65-71 (1995)). Repression of HER-2 by delivery of certain viral genes (adenovirus-5 E1a and SV40 large T antigen) into tumor cells in mice leads to suppression of tumor growth and longer survival of the mice (Hung et al. (1995), supra). Thus HER-2 is a very attractive candidate for antisense therapy, and c-myc is a useful marker for the most dangerous cells. Cancer differs from infectious disease in that the “infectious agents” are in most respects like normal cells; hence the greatest challenge in cancer treatment is finding cytotoxic or cytostatic agents sufficiently specific for cancer cells that they have minimal toxicity at therapeutic levels. We present here a novel approach to achieving the needed specificity by having two levels of recognition, analogous to a binary weapon. Thus, instead of attacking all cells that, say, are actively dividing, or that possess a particular cell-surface marker, the proposed agents down-regulate an appropriate target gene only upon binding to some molecule that is present mainly or exclusively in the cancer cell. The great power of the method is that the triggering molecule and the target gene can be (but are not required to be) completely unrelated. The target gene can potentially be any gene, even a housekeeping gene, although the highest level of specificity is achieved by choosing a target gene that is active mainly or exclusively in cancer cells. Here, we choose as the triggering molecule the phosphoprotein product of the c-myc gene, and HER-2 as the target gene. The mechanism of triggering is based on the padlock idea but where topological linkage requires stabilization by binding of a separate agent, the “clasp.” We propose to use c-myc as the clasp.

A novel feature of this two-tiered approach is that its effectiveness should be proportional to the product of the concentrations of two elements that are more abundant in breast cancer cells: c-myc protein and HER-2 mRNA (or DNA, in the antigene version of the approach). Since the HER-2 gene is often amplified by as much as 10-fold, and the abundance of c-myc may be similarly elevated in some cancer cells, the therapeutic index of our approach is expected to be much larger than current therapies.

This approach lends itself well as an adjunct to other, standard therapies. Because c-myc overexpression is associated with poor prognosis of breast cancer, it might be expected that the resistance of tumor cells to standard chemotherapeutic agents may correlate with c-myc expression. This was found to be the case with cis-platin: Treatment of cis-platin-resistant cells in culture with c-myc antisense oligonucleotides reversed the resistance, possibly by increasing uptake of cis-platin; and even in nonresistant cells that express c-myc, there was a synergistic cytotoxic effect (Mizutani et al., Cancer 74:2546-2554 (1994)). Synergistic effects have also been seen for combination c-myc-P53 antigene therapy (Janicek et al., Gynecol. Oncol. 59:87-92 (1995)).

Like other members of the myc family, c-myc functions in cell proliferation and differentiation (Vastrik et al: Crit. Rev. Oncog. 5:59-68 (1994) and Penn et al., Semin. Cancer Biol. 1:69 (1990)). It has been shown to be both necessary and sufficient for normal resting cells to enter the cell cycle. In some circumstances, it also mediates apoptosis (Cherney et al., Proc. Natl. Acad. Sci USA 91:12967-12971 (1994), Saito and Ogawa, Oncogene 11:1013-1018 (1995) and Shi et al., Circulation 88:1190-1195 (1993)). A phosphoprotein, c-myc is localized to the nucleus and is involved in the transcriptional regulation of several genes, including ornithine decarboxylase, p53, prothymosin α and ECA39. It forms a heterodimer with a related protein, max, through their homologous helix-loop-helix and leucine zipper domains; and the dimer binds to DNA much more sequence-specifically than does either protein alone (Blackwood and Eisenman, Science 251:1211-1217 (1991)). The heterodimer transactivates target genes through binding to the sequence CACGTG. Transactivation is relatively insensitive to orientation or position of this sequence relative to the gene being activated (Packham et al., Cell Mol. Biol. Res. 40:699-706 (1994)).

A number of studies have documented effects of treating cells with antisense oligonucleotides directed against c-myc. For example, treatment of T-cell hybridomas interferes with activation-induced apoptosis (Shi et al. (1993), supra). Treatment of colonic carcinoma cells inhibits their colony-forming capacity (Collins et al., J. Clin. Invest. 89:1523-1527 (1992)). Treatment of estrogen-dependent (MCF-7) breast cancer cells with 10 μM c-myc antisense oligonucleotide resulted in 95% inhibition of c-myc protein expression, a 75% reduction in estrogen-stimulated cell growth, and a cytostatic effect also on estrogen-independent MDA-MB-231 cells (Watson et al., Cancer Res. 51:3996-4000 (1991)). Similar reduction in c-myc levels and growth inhibition was achieved at lower oligonucleotide concentrations (<1 μM) by conjugation with poly(L-lysine) (Degols et al., Nucleic Acids Res. 19:945-948 (1991)). Antigene, triplex-forming oligonucleotides complexed with polyamines have been shown to suppress c-myc expression in breast cancer cells (Thomas et al., Nucleic Acids Res. 23:3594-3599 (1991)).

An oligonucleotide designed to form a triplex with a G-rich sequence in the promoter region of HER-2 has been shown to inhibit transcription factor binding (Noonberg et al., Nucleic Acids Res. 22:2830-2836 (1994)) and transcription in vitro (Ebbinghaus et al., J. Clin. Invest. 92:2433-2539 (1993)).

Full-length c-myc binds sequence-specifically to its target site only as a heterodimer with the related protein max (Blackwood and Eisenman (1991), supra). To avoid having to use both c-myc and max in our cell-free assays, we will take advantage of the fact that a truncated version of c-myc consisting of the basic, helix-loop-helix, and leucine zipper domains binds to the same site as the c-myc/max dimer.

Candidate antisense constructs will be tested for c-myc-dependent binding and translation inhibition first in a cell-free system and then tested for biological effects on cultured breast cancer cells. The goal of these prototype experiments will be a general procedure for down-regulating any gene in the presence of any given protein.

To accomplish the above, the following will be performed. First, the sequences HER-5′, HERMYC1, and HERMYC2 as shown in FIG. 8 will be synthesized in both phosphodiester and phosphorothioate forms. If finding the right conditions for making the padlock highly sensitive to the presence of the c-myc clasp turns out to be difficult, we will synthesize an analogous oligonucleotide containing the E. coli lac operator sequence in place of the c-myc-max binding site. This will permit optimization to be done with a single, easily available protein trigger, the lac repressor. If necessary, the lengths of linker and helical regions will be optimized by in vitro selection. The optimal lengths of helical segments determined for the lac repressor are likely to be also optimal for c-myc used as the clasp.

Next, the binding characteristics and kinetics by polyacrylamide gel-shift assay using 5′-³²P-labeled padlock oligonucleotides will be verified. The DNAs will be annealed by heating and slowly cooling to 2° C., then electrophoresed at different temperatures ranging from 4° C. to 37° C. in the presence of MgCl₂ to mimic intracellular concentrations of available Mg²⁺.

Next, the stability of the complexes will be verified by measuring the melting temperatures of padlock oligonucleotide HERMYC1 with target HER-5′ and HERMYC1 with HER-5′ by UV absorbance, ramping the temperature from 2° to 45° and back down at 0.5°/min in 25 mM Tris HCl (pH 7.4), 100 mM KCl, 1 mM EDTA. We will repeat in the presence of truncated c-myc protein (2, 10, and 100-fold stoichiometric excess). If the Tₘ is not above 37° in the presence of c-myc and below 20° in its absence, we will carry out an in vitro selection on the partially randomized HERMYC1R and HERMYC2R as shown in FIG. 8, to optimize the length of the helical segments in order to achieve this differential. If necessary, we will adjust the lengths of the cytosine linkers and/or replace with oligo(ethylene glycol) linkers (after in vitro selection).

Next, a T7 promoter fused to HER-5′ will be inserted into the NcoI site of pGL3 Control Vector (Promega, Madison, Wis.), a luciferase vector containing an SV40 promoter and enhancer. We will perform in vitro translation and 5′ capping using T7 RNA polymerase and capping reagent (Ribomax kit, Promega). We will then test the ability of HERMYC1 and HERMYC2 to block in vitro translation of the resulting RNA (as monitored by luciferase-dependent light production) using a rabbit reticulocyte lysate system, in the presence and absence of truncated c-myc. We expect truncated c-myc-dependent blockage in both cases.

Because several parameters may need optimizing for the padlock/target complex to have the best balance of low stability in the absence of the clasp and high stability in its presence, it may prove advantageous to select the best sequences from a randomized library of RNA sequences. Therefore, the selection may follow the scheme shown in FIG. 9. The target strand is single-stranded DNA (for stability) corresponding to the sequence of the target region of HER-2 mRNA. The DNA will be synthesized with a 5′-biotin tag and immobilized on a column of streptavidin agarose beads (or other matrix if higher heat stability is needed). Polymerase chain reaction (PCR) primer binding sequences are the standard forward and reverse sequencing primer sequences for plasmid pUC18 (New England Biolabs). The randomized region will consist of the region in bold in FIG. 9 (top) and marked n in the sequence (FIG. 9, bottom): 7 nucleotides on either side of the stem and 7 more in the stem, except that the ends of the sequence shown in bold will be fixed to provide an initial impetus to fold in the manner shown. This pool of partly randomized DNA will be incubated with the immobilized DNA target in the presence of a truncated c-myc protein. The unbound portion of the pool will be washed off the column, and the bound DNA will be recovered by mild heating (if necessary in the presence of 0.1% SDS). DNAs that bind in the absence of c-myc (thus bypassing the clasp) will be eliminated by passing this selected pool through the column once again in the absence of c-myc and collecting the flow-through. This depleted pool will be amplified by PCR and then subjected to further rounds of selection. The concentration of c-myc will be reduced gradually to increase the selection pressure for ligands for which c-myc has a strong “clasping effect.” The pool will be sequenced en masse to see whether the search is narrowing, and when specific sequence patterns emerge the pool will be cloned, individual colonies sequenced, and consensus sequences derived. These will be individually synthesized and tested for their ability to function as switches.

In vitro translation assays for ribozyme obstruction will be performed as described in the above example, using a fusion construct between HER-2 mRNA and luciferase. Controls will include oligos having scrambled and sense sequences in place of the antisense sequence, and clasp sequences that cannot bind c-myc. Before addition of an rabbit reticulocyte lysate translation mixture, oligos will be incubated in 50 mM Tris acetate (pH 7.5)/10 mM magnesium acetate, either alone or in the presence of a 10- to 40-fold molar excess of in vitro-transcribed (T7) HER-2-luciferase fusion constructs. Luciferase will be assayed according to the instructions from the translation kit manufacturer (Promega). A T7 promoter-luciferase expression plasmid lacking the target sequence will be used as a further control.

For cell testing, we will use the phosphorothioate versions of the successful oligonucleotides from the previous experiments. We will then expose estrogen-dependent MCF-7 breast cancer cells or other appropriate c-myc-overexpressing cells to this oligonucleotide or control oligonucleotides with and without estrogen, and assay for HER-2 mRNA, protein production, and cell proliferation. If appropriate, we will compare with estrogen-independent MDA-MB-231 cells, which constitutively express high levels of c-myc. We will use a cationic lipid vehicle: lipofectamine or lipofectin (Life Technologies), or dioleoylphosphatidylethanolamine (DOPE) together with a cationic cholesterol derivative (DC cholesterol) previously shown to be less toxic for down-regulation of HER-2 expression through a gene therapy approach (Hung et al. (1995), supra). We will also try the unmodified DNA oligonucleotides, since in some cases they survive intact long enough to be effective for treating cells in vitro.

Our clasp-dependent translation blockage can also be applied to blocking transcription; i.e., as an antigene agent. We will test the antigene capabilities of our locking system by targeting the HER-2 promoter region between the CAAT box and the TATA box, at the following sequence:

CACAGGAGAAGGAGGAGGTGGAGGAGGAGGGCT
GTGTCCTCTTCCTCCTCCACCTCCTCCTCCCGA
3′-TGGTGTTGGTGGTGGTGGTGGTGGTGGG-5′

which has been shown to inhibit transcription factor binding (Noonberg et al., Nucl. Acids. Res. 22:2830-2836 (1994)) and transcription in vitro (Ebbinghaus et al., J. Clin. Invest. 92:2433-2539 (1993)). Delivery and assays will be the same as for the antisense oligos. Attractive alternative target genes include H-ras and VEGF. A proposed padlock for the latter is shown in FIG. 1G, with a single helical turn providing linkage and a c-myc binding site. VEGF inhibition is likely to be helpful for growth control of a wide variety of tumors. An important advantage of this approach is that the padlock will act as a decoy “soaking” up large amounts of c-myc, which itself will reduce cell proliferation.

### EXAMPLE VI

### Target-Activated RNA Catalysis for Nucleic Acid Detection

With the rapid increase in available DNA sequence information, nucleic acid-based diagnostics is a subject of intense interest. Improvements in the efficiency of sample preparation, nucleic acid amplification, and detection would permit greatly increased use of such methods for routine diagnostic purposes. This example describes ways in which target binding can activate RNA catalysis, leading to improved methods of detection or amplification of target molecules. The most widely used method for amplifying DNA prior to detection is the polymerase chain reaction (PCR). Alternative isothermal methods involve a sequence of enzymatic steps, with the attendant complexity, costs of the protein enzymes, and risk of contamination during multiple tube openings. Here we propose innovations in isothermal amplification based on the use of RNA as an amplifiable probe and the ability of RNA to catalyze its own cleavage and rejoining reactions. By taking advantage of RNA catalysis, we can eliminate requirements for all protein factors except an RNA polymerase and reduce the number of tube, openings. Additional innovations in hybrid capture and wash procedures further increase the speed and ease of automation. The procedure lends itself to closed-tube, multiplex fluorescent detection during amplification, permitting rapid screening of many different targets while minimizing risks of exposure to pathogens or laboratory contamination by amplified targets.

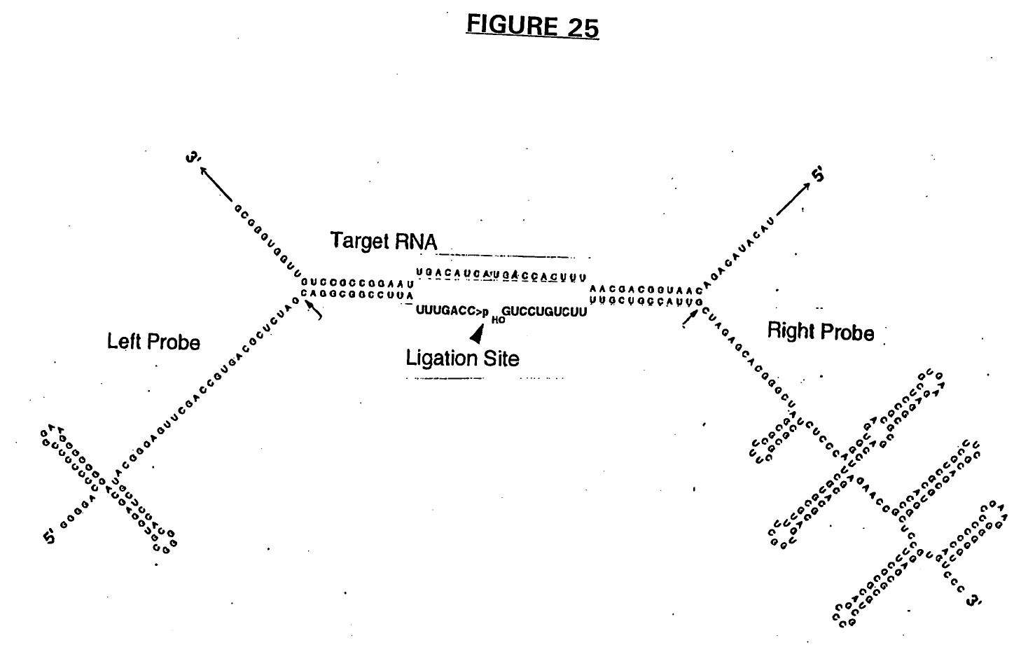

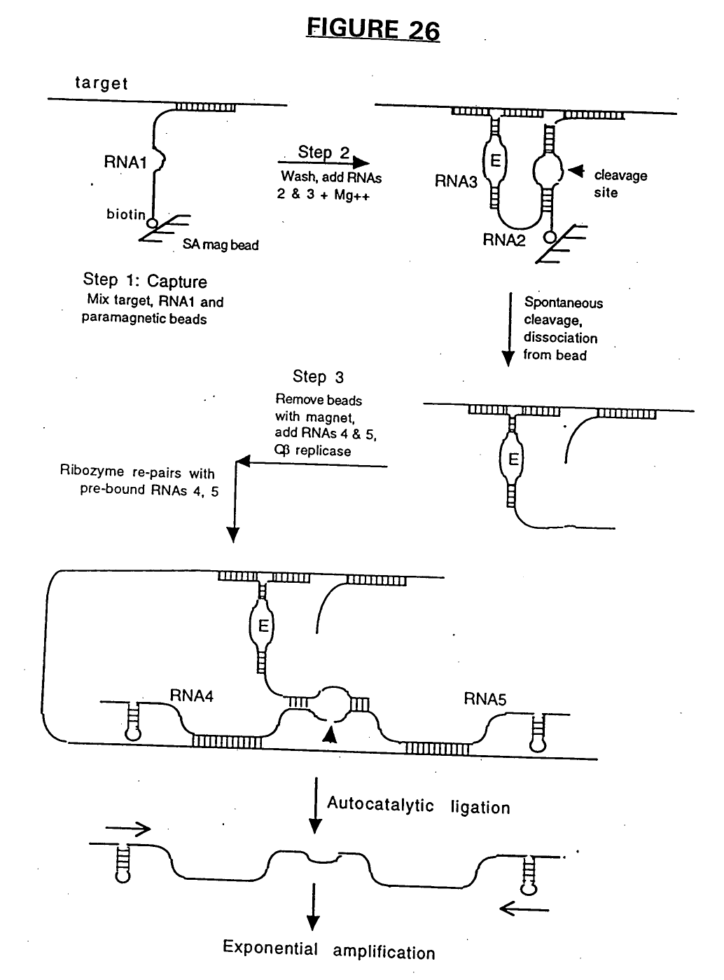

The ultimate goal is to design a scheme for nucleic acid-based diagnostics that can be used to detect either DNA or RNA, is sensitive, rapid, requires no thermal cycling, and readily lends itself to automation. Our proposed scheme is based on the use of RNA catalysis and Qβ-replicase for amplification. This RNA-dependent RNA polymerase will catalyze exponential replication of an RNA molecule possessing appropriate end sequences, without the need for primers or thermal cycling. Following the work of Tyagi et al., Proc. Natl. Acad. Sci. USA 93:5395-5400 (1996), we separate the end sequences by dividing the substrate RNA into two halves, called replication probes; hence amplification can proceed only if the two replication probes are ligated together (FIG. 25). They are designed to hybridize to adjacent sites on the target RNA, so that ligation (and therefore amplification) are dependent on the presence of the target. Background is minimized by capturing the target on a solid substrate through hybridization with capture probes, followed by washing away non-hybridizing RNAs and then release from the substrate. The purified target is hybridized to the replication probes, which are then ligated and amplified. In one embodiment, this procedure requires three enzymes: ribonuclease H(RNase H) to release from the solid support, DNA ligase (which acts on double helical RNA) to ligate the replication probes, and Qβ-replicase. In our proposal we utilize target-dependent RNA catalysis to substitute for both the nuclease and ligase proteins (FIG. 26). By incorporating their catalytic functions into the RNA probe molecules, we eliminate the need for any protein enzymes other than the replicase, and increase the specificity of target recognition by using shorter hybridization probes. We use the hairpin ribozyme for this purpose because it efficiently catalyzes both cleavage and ligation.

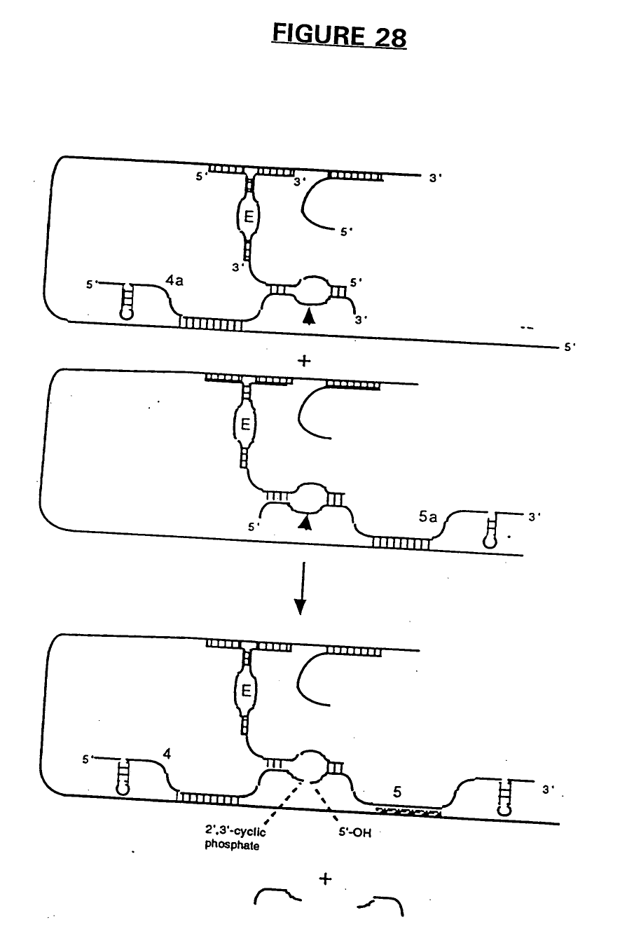

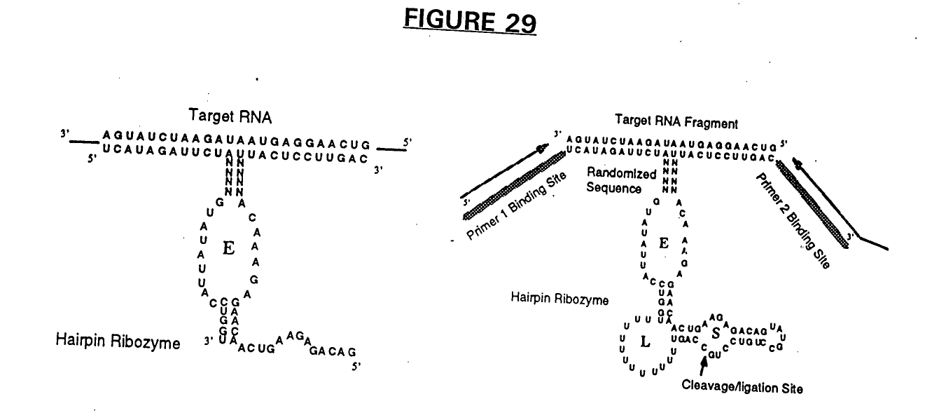

In light of the above, we will design and construct, using randomization and selection procedures, a latent hairpin ribozyme derivative becoming catalytically active only after binding to a target sequence (FIG. 29), demonstrate its ability to cleave a separate RNA (in trans) if the two RNAs are hybridized to adjacent sequences on a target RNA, demonstrate its ability to ligate a separate RNA if the two are hybridized to nearby sites, and demonstrate amplification of replication probes by Qβ-replicase following ligation by tethered ribozyme (FIG. 26). We will also use 5′-biotinylated RNA capture probes and demonstrate capture of the target molecule on streptavidin-coated paramagnetic bead, demonstrate release of captured target upon hybridization of ribozyme RNAs to adjacent site (FIG. 28) and demonstrate ability of target-hybridized ribozyme to ligate replication probes (FIG. 26). The lengths and/or AT/GC composition of helical stems surrounding cleavage/ligation sites will be adjusted, if needed. Together with other innovations in sample handling and detection, this approach will result in a significantly simpler, cheaper and faster procedure for nucleic acid detection.

Nucleic Acid-Based Diagnostics. The detection and quantitation of DNA and RNA are increasingly important techniques. They are used for diagnosing infectious diseases caused by viruses and microorganisms, detecting and characterizing genetic abnormalities, and identifying genetic changes associated with cancer or various types of treatment. Further uses are in detecting pathogenic organisms in the medical supply (such as blood banks), food and environment samples. As research tools these techniques are used in genetics, virology and microbiology, as well as in forensic sciences, anthropology and archeology. With their increasing importance comes a need for their simplification and improvement in order to be used routinely in many varied applications.

A common method for detecting genomic material is to quantitate specific nucleic acid sequences through hybridization with nucleic acid probes. These probes can carry radioactive or other types of labels, including ligands, which can interact with detecting moieties, e.g. streptavidin or digoxigenin. However, the sensitivity of nucleic acid hybridization is limited by the specific activity of the probe. Even under optimal conditions, direct hybridization methods can detect only down to 106 nucleic acid molecules (Horn and Urdea, Nucl. Acids Res. 17:6959-6967 (1989), which is not sufficient for many desired applications. Moreover, the most sensitive hybridization assays usually lack features required for routine applications—safety, economy, convenience and speed.

One solution of this limited sensitivity is exponential amplification of the target sequence. This can be carried out by either temperature-cycle assays such as the polymerase chain reaction (PCR) and ligation chain reaction, or isothermal procedures such as transcription-mediated amplifications and the restriction nuclease/DNA polymerase method. Alternatively, hybridization can alter another component of the reaction so as to make it amplifiable; examples include linear amplification methods such as induction of an enzyme reaction to produce a fluorescent product, or exponential amplification of reporter RNA through the use of Qβ-replicase (Chu et al., Nucl. Acids Res. 14:5591-5603 (1986)).

In practice, PCR, although providing very high sensitivity, has several limitations: (1) the occurrence of false positives generated by hybridization of primers to homologous sites in non-target DNA, (2) the presence of PCR inhibitors in specimens, and (3) its inability to directly amplify RNA due to its thermal lability. These drawbacks, together with market considerations of license fees for the use of patented PCR technology and the significant cost of thermal cyclers, have stimulated a search for alternatives. Prominent among the newer techniques is the isothermal, exponential amplification of recombinant RNA probes by Qβ-replicase. Although the PCR and the Qβ-replicase assays have similar sensitivity (Lizardi et al., Biotechnology 6:1197-1202 (1988)), the Qβ-replicase assay is simpler, faster and less expensive than PCR. The substrate for Qβ-replicase is RNA rather than DNA, and RNA combines the dual functions of hybridization probe and amplifiable reporter. In this proposal we make use of a unique capability of RNA, autocatalysis, to eliminate the need for two protein enzymes used in a promising current amplification scheme (Tyagi et al. (1996), supra) employing Qβ-replicase.

Qβ-replicase is an RNA-dependent RNA polymerase from the coliphage QB. It is capable of replicating the single-stranded Qβ RNA genome in infected cells while ignoring the huge excess of bacterial RNA, and similar specificity has been observed in in vitro assays (Haruna and Spiegelman, Science 150:884-886 (1965)). As little as one molecule of template RNA can in principle initiate its exponential amplification by Qβ-replicase without any need for primers. The first step is the template-directed synthesis of the complementary strand, after which the two complementary strands spontaneously separate, permitting each to be the template for another round of complementary strand synthesis. As long as there is a stoichiometric excess of the enzyme, the number of replicated RNA molecules increases exponentially. After the number of RNA replicas equals the number of enzyme molecules, the RNA amplification continues linearly. The final amount (more than a billion copies) of the synthesized RNA (typically, 200 ng in 50 μl in 15 min at 37° C.) is so large that it can be easily detected by simple colorimetric techniques (Lizardi et al. (1988), supra.

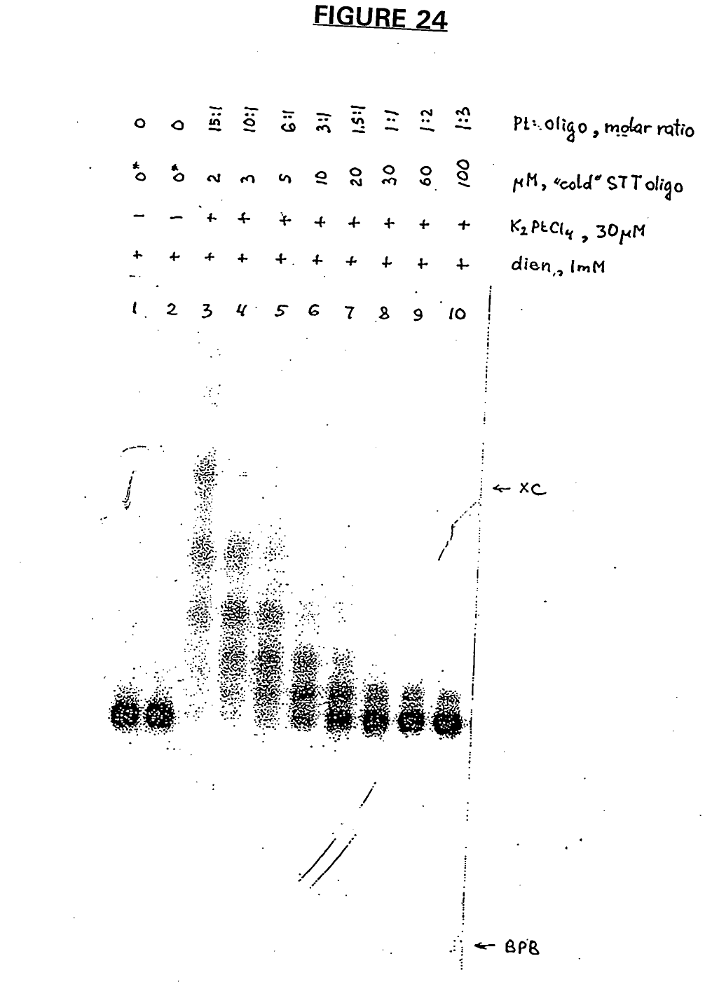

Other than the natural Qβ genome, MDV-1 RNA, only a few RNAs have been found to exhibit template activity for Qβ-replicase (Munishkin et al., J. Mol. Biol. 221:463-472 (1991)). Although Qβ-replicase has been studied for more than 30 years, the precise RNA structure requirements for replication remain obscure. The only obvious common element is extensive secondary structure within the single strands, which probably serves to prevent or inhibit duplex formation after replication (see FIG. 24); RNA double-stranded complexes cannot serve as templates (Brown and Gold, Biochemistry 34:14775-14782 (1995)). In vitro, in the absence of appropriate templates, Qβ-replicase can spontaneously synthesize short RNA species (30-45 nt in length) having random sequences (Biebricher et al., J. Mol. Biol. 231:175-179 (1993)). However, since this template-free synthesis starts after long lag times and proceeds much slower than the template-directed RNA amplification (Biebricher et al. (1993) supra), this secondary process does not produce a complication for the Qβ-replicase amplification assay.

Two developments led to the possibility of using Qβ-replicase, despite its highly specialized template requirements, for amplification of arbitrary RNA sequences. First is the discovery that an oligoribonucleotide fragment (up to 58 nt in length) can be inserted within the sequence of 221-nt MDV-1 (+) RNA (a naturally occurring template for Qβ-replicase) between the nucleotides at positions 63 and 66 without interfering with its replicability (Lizardi et al. (1988), supra). This capability permits the incorporation of functions in addition to being an amplifiable reporter. The second development is the design of latent RNA probes which cannot be amplified unless they hybridize to their target (EP-A-707 076 and Tyagi et al. (1996), supra), so-called “smart” probes. These permit very low background in hybridization assays, since signal generation is strictly dependent on the presence of target RNA.

One smart probe approach is to divide the amplifiable reporter RNA into two separate molecules neither of which can be amplified by itself, because neither contains all the elements of sequence and structure that are required for replication. The division site is located in the middle of the embedded probe sequence. When these “binary probes” are hybridized to adjacent positions on their target, they can be joined to each other by incubation with an appropriate ligase, generating an amplifiable reporter RNA. Nonhybridized RNA probes on the other hand, because they are not aligned on a target, have a very low probability of being ligated.

In another approach (EP-A-707 076), latent Qβ-replicable template has been created by extending on the 5′ end of MDV-1 RNA resulting in inhibition of its replication by Qβ-replicase. A ternary hybrid formed between this latent substrate, a second RNA probe, and a target RNA produces an autocatalytic RNA structure (hammerhead ribozyme) which cleaves the 5′ extension from the latent template in the presence of divalent cations, thereby converting it to an efficiently replicating form and effecting its release from the support. This approach is interesting as a first attempt to use the catalytic potential of RNA molecules in nucleic acid-based diagnostics. However, it has two disadvantages: It is limited to target sequences that contain the element GAAA (required to generate an active hammerhead ribozyme) and unintended spontaneous RNA cleavage (and, therefore, activation of replication) can readily occur through transesterification, catalyzed by the same divalent metal ions that are required as catalytic cofactors for the ribozyme.

In this approach, we combine the best features of these two approaches by employing a hairpin ribozyme construct for the target-dependent-ligation of two halves of a split substrate for Qβ-replication. Our approach imposes no sequence restrictions on the target RNA, and because spontaneous ligation can be made much rarer than spontaneous RNA cleavage, the background of false positives should be very low. Moreover, because the hairpin ribozyme can catalyze both ligation and cleavage (see below), it can play dual roles of target-dependent ligase and specific endonuclease. We use the endonuclease activity for release from a solid support after hybrid capture, replacing a ribonuclease H(RNase H) used in the Tyagi et al. procedure. (As mentioned above, capture permits a purification step prior to amplification, which reduces background.) We further suppress background by requiring that each step of the procedure be dependent on recognition of the target; this is accomplished through hybridization with five separate RNAs. Actually, this approach works for both RNA and ssDNA targets.

The HPR has distinct domains for substrate binding and catalysis that interact through specific tertiary contacts (see above). These domains can be on the same RNA molecule or on separate molecules (Feldstein et al., Gene 82:53-61 (1989)). The release of cleavage products permits binding to another substrate for further cleavage or ligation. Both HPR and Qβ-replicase require magnesium ions for activity in dilute solution.

Ability of HPR to cleave and ligate adjacent, distant, and unattached substrates. We have demonstrated the ability of two HPR constructs, HPR1 and HPR2, to cleave and ligate adjacent or distant substrate sequences on the same RNA strand (reaction in cis), as well as to ligate substrates on separate molecules (reaction in trans) through formation of specific RNA-RNA complexes. A 150-nt DNA template encoding the T7 promoter, a spacer sequence, and the pre-processed sequence of the minimonomer hairpin ribozyme was transcribed to generate the pre-HPR1 RNA (161-nt). This template was transcribed by T7 RNA polymerase in the presence of the non-radioactive nucleoside triphosphates and/or [α]³²P] CTP. Denaturing polyacrylamide gel electrophoresis of transcription products revealed the presence of several RNA species (FIG. 2E). These species were identified as unprocessed linear RNA, semi-processed linear RNA, fully processed linear RNA, fully processed circular HPR1, and linear HPR1 dimer (supra). All processing events were the result of autocatalytic cleavage reactions of the single ribozyme moiety. These experiments highlighted the ability of a single catalytic domain to cause cleavage at both an adjacent site and a site tens of nucleotides distant. Other experiments showed that ligation resulted in the presence of multimers, indicating that a catalytic domain from one HPR molecule catalyzes its joining to a separate molecule. This ability of a single catalytic domain to catalyze both cleavage and ligation of nonadjacent substrates is utilized in our scheme as diagramed on FIG. 25 and described below.

Catalysis by freezing and ethanol. Extensive characterization of individual forms generated by self-processing of HPR T7 transcripts revealed that either freezing alone or exposure to >40% ethanol alone results in ligation of linear forms in the absence of divalent cations (Kazakov et al., submitted)). Actual freezing was required, since ligation did not occur in supercooled but unfrozen solutions at −21° C.

Both freezing and ethanol treatment were able to produce yields of ligated product (up to 85%) that exceed the yield (˜50%) in normal aqueous solution. Since the key step in our scheme for amplification by Qβ replicase is ligation of the replication probes (see below), we have the option of incorporating a freezing step if needed for maximal sensitivity.

We describe here procedures for analysis of sample of cells to be analyzed for pathogens through detection of mRNA; with an initial denaturation step, a similar protocol would work for DNA targets, suitable, for example, for detection of genetic variants. The general scheme is outlined in FIG. 26. The first step involves capture of the target on a solid substrate using a probe that is complementary to the desired target, so-called hybrid capture. The procedure for hybrid capture is analogous to that used by Tyagi et al. (1996), supra, except for the composition of buffer solutions and the structure of the capture probe. The capture probe consists of a sequence complementary to the target RNA, a short linker, the substrate sequence for the HPR, another linker, and a terminal biotinylated nucleotide (FIG. 26). It needs to be composed of RNA residues in the region of the cleavage site; the rest can be DNA or a nuclease-resistant analog. In our first version, it will be all RNA for simplicity.

A sample of cells to be analyzed for pathogens is dissolved by incubation in 5 M guanidine thiocyanate for 60 min at 37° C. This treatment lyses cells, inactivates enzymes, frees DNA and RNA from intracellular structures, and weakens RNA secondary structures (Pelligrino et al., Biotechniques 5:452460 (1987)). Lysates are adjusted to reduced guanidine thiocyanate concentration (Buffer A: 2M guanidine thiocyanate, 400 mM Tris-HCl (pH 7.5), 0.5% sodium N-lauroylsarcosine, 0.5% BSA); this solution continues to block nuclease activity and promotes RNA-RNA hybrid formation without interference from cell debris (Tyagi et al. (1996), supra). Capture probe RNA1 is added and allowed to hybridize for 60 min. The target RNAs complementary to the capture probes are then captured by adding 20 μl of a suspension of streptavidin-coated paramagnetic particles (Promega) and incubating at 37° for 10 min (FIG. 26, Step 1). The presence of 2 M guanidine thiocyanate does not interfere with binding of the biotin group to streptavidin (Tyagi et al. (1996), supra).

The noncomplementary nucleic acid molecules, excess capture probes, and cellular debris are removed by thorough rinsing, first with Buffer A (4 times), then with Buffer B (5 mM MgCl2, 66 mM Tris-HCl (pH 7.5), 0.5% Nonidet P-40 (Sigma)) to remove guanidine thiocyanate (4 times). For each wash cycle, the mixture is vortexed, and beads are gathered to the side of the tube with a magnet, and the solution is removed and replaced with a fresh wash.

After the last wash step, a final aliquot of Buffer B is added along with RNAs 2 and 3 (FIG. 26, Step 2). These RNAs comprise most of the ribozyme's catalytic domain E (plus its substrate binding sequence) but, in place of part of an essential helical stem, they contain sequences complementary to adjacent regions of the target RNA. Sargueil et al., Biochemistry 21:7739-7748 (1995) showed that when this stem is too short, the ribozyme is unstable and catalytically inactive, but its activity could be increased to a level even greater than that of the native ribozyme by lengthening the stem. In our system, the short stem is stabilized upon hybridizing of its ends to the target, thus creating the active conformation of domain E. The substrate-binding domain of the HPR then pairs with the HPR substrate sequence on the capture probe, leading to rapid cleavage of the latter with at least 50% efficiency. Because the off-rate for binding of half-substrate sequences is rapid, the complex will be dissociated from the bead, and domain E will be available for a new substrate in Step 3.

To remove from further reaction any excess capture probes and uncleaved complexes, the beads are drawn aside with a magnet and the solution is transferred to another tube containing RNAs 4 and 5, buffer C (15 mM MgCl₂, 45 mM Tris-HCl (pH 8), 100 μM ATP, 600 μM CTP, 600 μM UTP, and 600 μM GTP) and Qβ-replicase (6 μg, Vysis).

From this point forward, no further manipulations are required; the remaining steps proceed in sequence automatically. RNAs 4 and 5 hybridize to a pair of sites located 500 nt from the binding site of the HPR domain E, and the 2′-3′-cyclic phosphate end of RNA4 and the 5′-OH end of RNA5 make up a substrate pair that can bind and be ligated by the HPR. They can form a complex through looping of the target RNA as shown in FIG. 26. This complex mimics the structure of the native HPR in its cleaved form, and leads to ligation with an efficiency of approximately 50%.

Synthesis of RNAs with appropriate ends. The replication probes must have 5′-OH and 2′,3′-cyclic phosphate ends in order to be ligated by HPR domain E. The simplest way to achieve this is the automatic scheme shown in FIG. 28. Two RNAs, each containing the full HPR substrate domain but only one Qβ-recognition sequence, are provided to the target-hybridized domain E. Whenever one of these RNAs hybridizes to the target and occupies the substrate binding site it will be cleaved (FIG. 28). Due to the high off-rate for the cleaved products, these products will dissociate from domain E, permitting another uncleaved RNA to bind and be cleaved. After some time, enough RNAs will be cleaved that with reasonable probability a left-hand and a right-hand replication probe will bind to the same Domain E and since they will now have the appropriate ends, they will be ligated. Qβwill then rapidly amplify these molecules. The process is aided by the fact that the large cleavage products will remain near Domain E due to hybridization to the target, whereas the small fragments will diffuse away.

The above described scheme will be tested using as target a sequence from the pol gene of HIV-1. Domain E will bind to nt 4668-4682 of the HIV genome (Tyagi et al. (1996), supra), the capture probe will bind to nt 4716-4760, the left replication probe to nt 4577-4588, and the right to nt 4607-4618. We will first test for the efficiency of the individual steps by using ³²P-labeled target or probes and following the recovery at each step.

We must also design a derivative of HPR that has catalytic activity dependent on target binding. In the normal HPR, the catalytic domain (E) is stabilized by a short Watson-Crick helix capped by a loop. The loop can be replaced by three additional base pairs and full activity is maintained. However, other alterations of the stability of this helix can strongly affect both thermostability and catalytic activity of hairpin ribozyme. To make catalytic activity dependent on target binding, we substitute a Y-branch for the closed loop and select a sequence that will not support catalytic activity unless the target is accurately paired.

The scheme for selecting sequence NNNN is an in vitro selection and amplification procedure (Breaker and Joyce, TIBTECH 12:268-275 (1994)) based on sequential RNA-catalyzed cleavage and ligation reactions (Berzal-Herranz et al., Genes Dev. 6:129-134 (1992)). Two RNA molecules are synthesized that, in the presence of an oligonucleotide target fold into the hairpin ribozyme-like structure shown in the right panel of FIG. 29. The four nucleotides NNNN on each strand are randomized during chemical synthesis so that all possible sequence combinations are represented. The remainder are hybridized with the target and subjected to reverse transcription and PCR using primers complementary to the primer binding sites. The right-hand primer will have a 5′ extension consisting of a promoter for T7 RNA polymerase, permitting the amplified DNAs to regenerate a subset of the RNA pool by transcription. These RNAs will be hybridized to the target, and catalytically active sequences will be partially self-cleaved. The cleaved molecules will be isolated by denaturing gel electrophoresis and subjected to another cycle of selection for ligation in the presence of the target. Since only 256 different sequences are possible (2⁸), at most a few cycles should be sufficient to identify the best candidates from the pool. Negative selection against target-independent ligation will be carried out by several cycles of incubation without the target followed by gel purification of unligated molecules. Finally, the final PCR products will be cloned and sequenced. DNA templates for the dependent catalytic activity (both cleavage and ligation). Winners will be synthesized and individual RNA transcripts will be tested for the desired target-dependent catalytic activity.

An alternative method is to synthesize a series of molecules having from 0 to 6 A-U base pairs and 0 to 4 G-C pairs in place of NNNN, testing them individually for target-dependent catalytic activity.

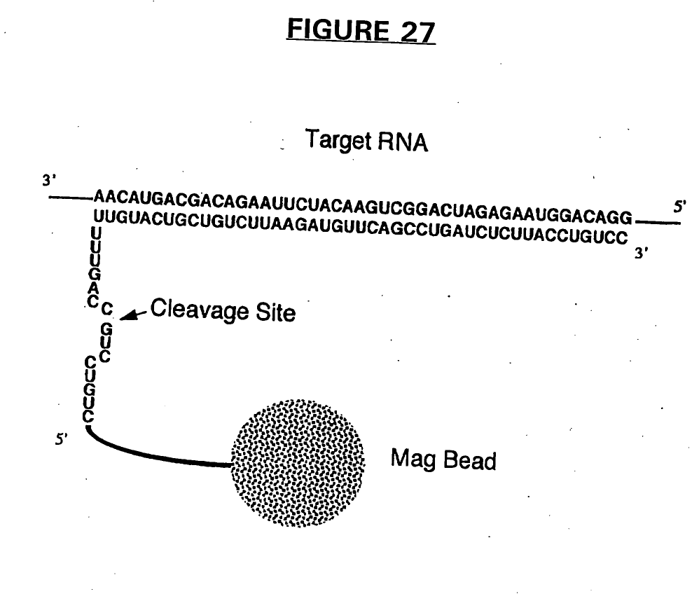

Having obtained good candidates for the target-dependent catalytic moiety, the next step will be to demonstrate its ability to cleave a separate RNA (in trans) if the two RNAs are hybridized to adjacent sequences on a target RNA. For this purpose, we will construct DNA templates for the transcription of target RNA spanning the region of HIV to which all the probes bind (nt 4500-4800). Using the biotinylated probe shown in FIG. 27, we will demonstrate capture of this (labeled) RNA on streptavidin-paramagnetic beads, following the procedures given above and in Tyagi et al. (1996), supra). Upon adding RNAs 2 and 3, we will test for release of the labeled RNA from the beads. If necessary to achieve release of a reasonable fraction of bound complexes (50% would be ample), we will adjust reaction conditions (temperature, magnesium ion concentration, etc.) and the length of the oligo(U) spacer on the capture probe (FIG. 27).

The next step is to test the ability of the target-tethered ribozyme to ligate the Qβ replication probes (constructed with appropriate ends as described above) and achieve amplification in the presence of the replicase. If necessary, we will test the ability of Qβ replicase to use our probes as substrates by synthesizing a short complementary RNA to hold the ends together and performing ligation with DNA ligase.

Finally, we will combine the above steps and test the ability of the ribozyme to switch from cleavage of the capture probe to ligation of the replicase probes in the presence of the target. If needed, we will adjust lengths and/or AT/GC composition of helical stems surrounding cleavage/ligation sites if needed.

To test for sensitivity and background, prepare simulated diagnostic samples by using a dilution series of T7-transcribed target RNA so that samples will contain as little as 1 molecule of target. α-[³²P]-CTP will be included in the Step 3 (see FIG. 25) and aliquots will be removed at 1-min intervals beginning 10 minutes into the reaction, until the reaction has proceeded for 35 min. Each aliquot will be precipitated by acid (addition of 400 μl of 360 mM phosphoric acid, 20 mM sodium pyrophosphate and 2 mM EDTA (Tyagi et al. (1996), supra). The precipitates will be collected a nylon membrane (Zeta-Probe, BioRad) through a vacuum manifold, washed, and quantitated by autoradiography or using a Phosphorimager (Storm 840, Molecular Dynamics).

For detection of HIV-1 pol gene RNA in mammalian cells, we will use a COS-like Monkey kidney cell line (CMT3) stably transfected with plasmids pCMVgagpol-rre-r (containing gag and pol genes) as a model (Anazodo et al., J. Clin. Microbiol. 33:58-63 (1995)). These cells contain a biologically active provirus that expresses HIV-1 pol mRNA. Simulated clinical samples will be prepared as a dilution series of cells expressing HIV-1 pol RNA in a constant population of nonexpressing cells. The number of expressing cells per 100,000 nonexpressing cells will range from zero and 1 to 10,000. Finally, actual clinical specimens will be tested. We also will test the effectiveness of the sample preparation procedure on blood and urine samples to see how well guanidine thiocyanate alone will protect against the effects of nucleases in those fluids. Additional nuclease inhibitors and/or the use of 2′-amino or other appropriate modifications to RNA probes will be employed if necessary.

As an alternative detection procedure, we will simply add a small amount of ethidium bromide or propidium iodide and follow the increase in fluorescence in a fluorometer as the Qβ reaction proceeds. Fluorescence detection will be the method of choice for commercial version of this method. The fluorophore can be either using a single intercalating dye as here, or a panel of oligonucleotides each conjugated to a different fluorophore distinguishable by their emission or excitation maxima. Each oligonucleotide would be complementary to a different replication probe and a different target, permitting multiplex amplification and detection of many different targets in the same sample. With appropriate reaction containers, the fluorescence could be measured without opening the container, reducing risk of contamination of the lab by amplified product and permitting destruction of the sample immediately after measurement.

Plunno et al., Anal. Chem. 67:2635-2643 (1995) have described a fiber-optic DNA sensor for fluorometric determination of nucleic acids, involving tethering of oligonucleotide probes to the surface of quartz fiber-optic filaments. Target nucleic acids hybridized to the probes cause enhanced fluorescence of intercalated ethidium bromide, which is detected through epifluorescence based on excition and emission through total internal reflection along the fiber. We plan to test the use of capillaries bearing tethered probes instead of streptavidin beads for hybrid capture. Unlike solid fibers, capillaries permit reaction and washing steps to be done without pipetting, much as DNA is synthesized on a solid-phase column. Detection could still be performed through fiberoptic means, since the capillary would provide for total internal reflection of exciting and emitted light at least as efficiently as with solid fibers.

Enhancement of ligation by freezing. The efficiency of ligation can be increased from about 50% to about 85% by freezing the solution to −5° for 15 min. We will try this procedure to see if the added efficiency warrants the additional step.

Relaxing stringency. The procedures described above provide the maximum level protection from false positive signals, by requiring independent target recognition by five separate RNA molecules in order for amplification to proceed. Such high “stringency” may not be necessary, especially for certain applications. Thus, it may not be necessary to have the replication probes bind to the target, as long as the catalytic activity of Domain is strictly dependent on target binding. The replication probes could be tethered to Domain E either covalently or via hybridization or metal coordination to provide efficient target-dependent ligation in this case.

Sensitivity. The sensitivity of this assay depends on the efficiency of all the steps leading to amplification of the target RNA. In the version of Tyagi et al. (1995), supra, the proportion of target molecules that resulted in amplifiable product was 2.5%. The main source of loss was the ligation step using T4 DNA ligase, which operates inefficiently on RNA and produced only 8% ligated product. In contrast, typical HPR derivatives such as HPR1 exhibit ligation efficiencies of 50% and as high as 85% upon freezing. Even with an overall efficiency of 2.5%, Tyagi et al. (1995), supra, could detect the presence of 100 but not 10 molecules of target. We anticipate that the higher yield of HPR ligation will increase the sensitivity to less than 10 molecules.

The other important characteristic of an effective diagnostic technique is the background of false positive events. False positives in this procedure come from ligation events that occur in the absence of hybridization to the target. Qβ-replicase can occasionally continue polymerization across a gap between replication probes that somehow are juxtaposed to each other, resulting in an amplifiable reporter RNA. Tyagi et al., (1995), supra, found that the only significant source of such events was the beads, which apparently sometimes bound the probes in such a juxtaposition. By including the capture step, concentration of free replication probes was so low that not even a single amplifiable RNA was generated from a sample containing 100 uninfected cells, although a single infected cell in 100,000 was detected. Our procedure retains this feature, and has the added security that to generate an amplifiable RNA molecule, four separate RNAs must be brought together by hybridization to a single target molecule. Domain C is catalytically inactive without some stabilizing interaction, such as with a binding protein (Sargueil et al., Biochemistry 21:7739-7748 (1995)) or our target. While there is some probability that a ligatable complex could occur between a target-bound domain C and RNAs 4 and 5 that were not bound to the target, the creation of such a complex would be a third-order event and the individual binding energies between partners are low. In any case, such unlikely events would not lead to false positives, because they still depend on domain C binding to the target.

All of our proposed nucleic acids are RNAs and hence are sensitive to cleavage by contaminating ribonucleases. The inclusion of 2 M guanidine thiocyanate in the cell lysate mixture was sufficient to prevent cleavage of target RNA to a degree that would significantly reduce the sensitivity of the assay. Since the lengths of RNA required in our procedure are comparable, this precaution is likely to be sufficient here also. If further reduction in nuclease cleavage is desirable, several options are available. Additional inhibitors of RNases, such as SDS, phosphate ions, and RNase inhibitors such as RNasin (Promega) could be included in the capture step. Also, RNAs 1-3 (and perhaps 4 and 5) can be synthesized with 2′ modifications such as amino groups that render them RNase resistant. These modified nucleotides can be present in all but a few positions and still permit efficient catalysis. Moreover, they can be synthesized by T7 RNA polymerase using appropriate nucleoside triphosphates.

We also may employ longer pairing stems in the substrate sequences for RNAs 4 and 5 and shorter ones for RNA 1, then adjust the temperature of incubation so that release of captured target is efficient while providing good yield of ligated replication probe. If necessary, we could employ a second Domain E, binding close to the binding sites of the replication probes, and also dependent on target binding for ligation activity.

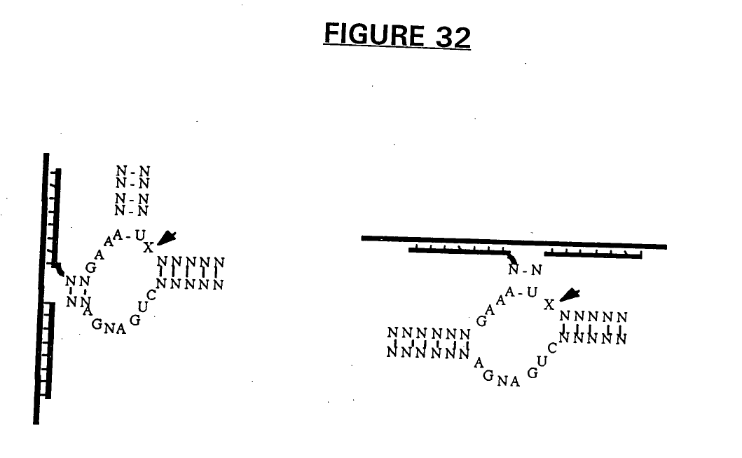

The above discussion illustrates one manner in which binding of a target can activate a latent ribozyme to produce a signal for detecting the target molecule. Another embodiment of this idea is shown in FIG. 32. Here binding of a nucleic acid target molecule stabilizes the structure of a hammerhead ribozyme, leading to cleavage of its substrate strand. Such cleavage can elicit a signal, as for example in the case of the left-hand construct, if the substrate strand is tethered to a solid support at one end and has a signal group at the other end, such as a fluorophore or biotin. Binding of a target molecule leads to separation of the signal group from the solid support, where it could be quantitated by standard methods upon removal from the solution of the solid support. Alternatively, one end may be attached to a fluorescent group and the other end to a quencher of fluorescence such that cleavage causes dequenching and fluorescence appears (Walter and Burke., RNA 3:392-404 (1997)). Because the target nucleic acid molecule does not have to be cleavable by the ribozyme, there are no limitations on its sequence.

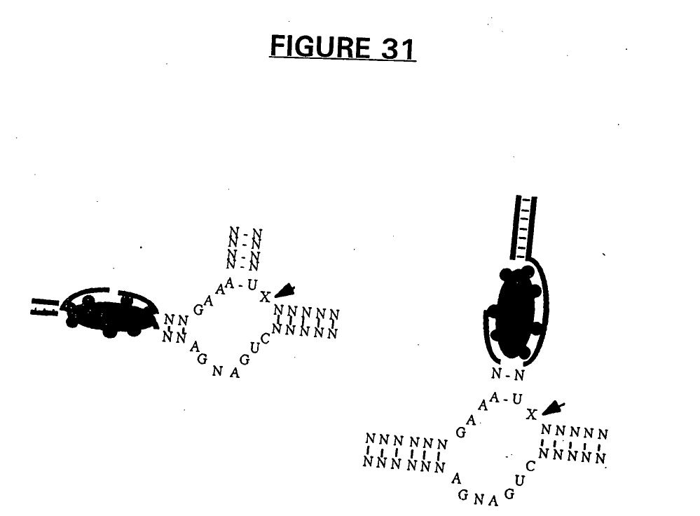

A more general embodiment of this idea is shown in FIG. 31. Here the target molecule can be potentially any molecule of interest, including proteins, small molecules, and metal ions. The binding site for the target molecule comprises the ends of two strands of the hammerhead ribozyme as shown; the sequences of those ends are selected from combinatorial libraries of DNA or RNA sequences to bind specifically and tightly to the target of interest (Tang and Breaker, RNA 3:914-925, 1997). Binding of the target molecule stabilizes the active conformation of the ribozyme and produces cleavage of the substrate strand.

### EXAMPLE VII

### Methods for Labeling Antisense and Triplex Forming Oligonucleotides with Platinum

All stock solutions were prepared in water deionized by a Milli-Q apparatus (Millipore), and then filtered through 0.22 Am filter units (Nalgene). The contents of common stock buffer and solutions used were as follows:
20% AUB: 19% acrylamide/1% N,N′-methylene-bis-acrylamide/8.8 M urea/1^{x}TBE.
FB: 0.5 M NH₄-acetate/0.1% SDS/1 mM EDTA
2×FLS: 92% formamide/10 mM EDTA/0.04% XC/0.04% BPB
10×PKB: 0.5 M Tris-HCl, pH 7.5/0.1 M MgCl₂/50 mM DTT/1 mM spermidine/1 mM EDTA
10×TBE: 445 mM Tris-borate, pH 8.3/12.5 mM EDTA
1×TE: 10 mM Tris-HCl, pH 8.0/1 mM EDTA
100×TAE: 100 mM Tris-acetate, pH 7.5/10 mM EDTA

Synthetic 16-mer oligodeoxyribonucleotide (TT) and its phosphorothioate derivatives (TST and STT) (1 micromole scale, GF grade) were obtained from Midland Certified Reagent.

| | |
|---|---|
| TT | d(TTCCTCTTTGGGGTGT) |
| TST: | d(TTCCTCTT_{S}TGGGGTGT) |
| STT: | d(T_{S}T_{S}T_{S}C_{S}C_{S}T_{S}C_{S}T_{S}T_{S}T_{S}G_{S}G_{S}G_{S}G_{S}T_{S}G_{S}T). |

Dried stocks of oligodeoxynucleotides were dissolved in 1×TE buffer to get a concentration about 10 μg/μl and then passed through the ‘Ultra free MC filter units’. 20 μl of these solutions were mixed with 20 μl of 2×FLS, loaded on the 20% denaturing polyacrylamide gel (1.6 mm), and then electrophoresed at 800 volts. The main oligonucleotide bands were located by UV shadowing of the gel, than cut out and extracted from the crushed gel slices by soaking into elution buffer (EU) at 37° C. for 2 h. The extract was passed through the microcentrifuge 0.22 μm filter, and the clear solution obtained was mixed with 4 vol. of absolute ethanol and kept overnight at −80° C. Precipitation of the purified oligonucleotides was completed by centrifugation 14,000 rpm at 4° C. for 10 min. Pellets were washed twice by 1 ml of absolute ethanol, dried, resuspended in 100 μl of 1×TE and passed through the microcentrifuge 0.22 μm filter. When not being in use, the oligonucleotide solutions were kept at −20° C. Aliquots (25 μl) of these solution were diluted to 1 ml and UV spectra were measured to determine their concentrations. The 5′-end labeling of the oligodeoxynucleotides using T4 polynucleotide kinase (Richardson, 1981) were done using labeling protocol described below:
1 μl 0.5 mM (2.5 μg/μl) gel-purified oligonucleotide
2 μl H₂O
1 μl 10×PNK buffer (Promega)
5 μl [γ-³²P]ATP (10 μCi/μl) (Amersham)
1 μl T4 Polynucleotide kinase (10 U/μl) (Promega)
Mix and incubate at 37° C. for 30 min.

³²P-labeled oligonucleotide species were purified (and analyzed) by electrophoresis through 20% denaturing polyacrylamide gels. Immediately before loading onto the gels, the solutions were mixed with equal volumes of 2×FLS, and heated for 2 min at 95° C. Individual oligonucleotide bands were located by autoradiography and isolated from the gels as described above.

Diethylenetriamine catalyzes platination of oligonucleotides. Various reaction mixtures (total volume 10 μl) were combined as following:
1 μl of ³²P-labeled oligonucleotide
14 μl of 150 μM of the same non-radioactive oligonucleotide
2 μl of 5-50×TAE buffer
0.3 μl of 0.01-1 mM K₂PtCl₄
0.3 μl of 0.1-15 mM dien in 10-100×TAE
0.3 μl of 0.5-500 mM NaCl, or 0.05-50 mM KI, or 0.075-7.5 mM DMSO, or 0.05-5 mM thiourea
0.7 μl of H₂O

The reaction conditions, including reagent concentrations, temperatures and times of incubation, are indicated in the figure legends. The platination reactions were stopped by the addition of 1 μl of 1 M NaCl (Brabec et al., 1994), mixed with an equal volume of 2×FLS and analyzed by electrophoresis on 20% denaturing polyacrylamide gel.

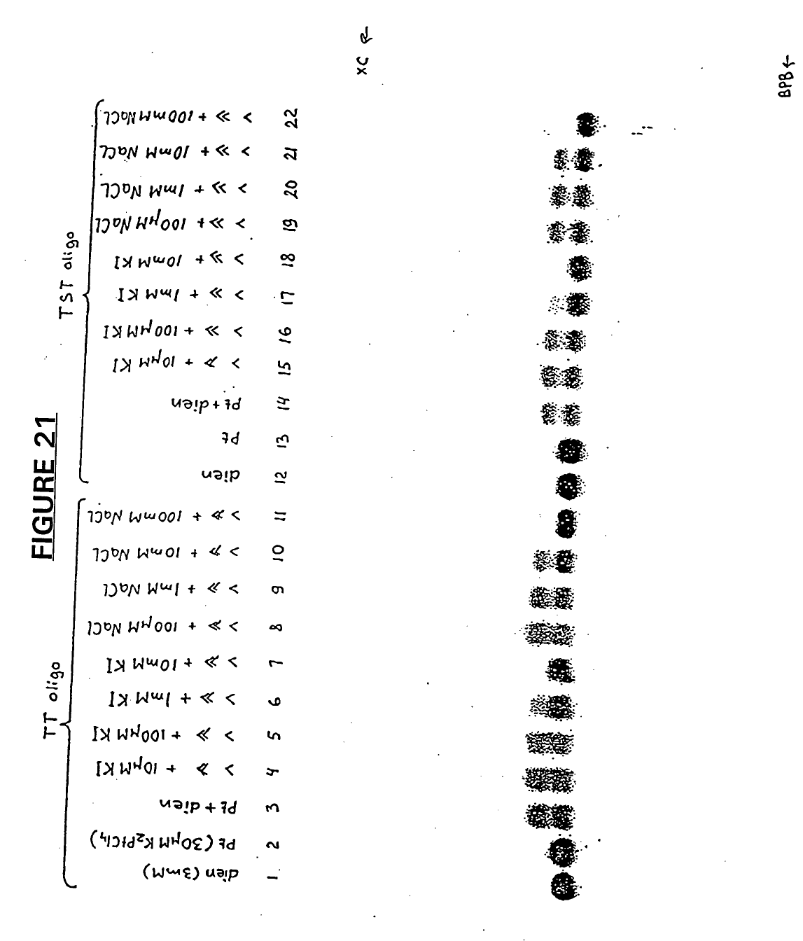

In agreement with literature data (Wherland et al., 1973; Kasianenko et al., 1995) regarding low reactivity of anionic [PtCl₄]²⁻ towards nucleic acids, we found that K₂[PtCl₄] at 30 μM concentration does not affect the electrophoretic mobility of the 10 μM oligonucleotide TT or its derivative TST having single phosphorothioate (POS) linkage (FIG. 21, lanes 2 and 13) after incubation for 1 h at 45 μC in 10×TAE [10 mM Tris-OAc (pH 7.5), 1 mm EDTA. Cationic diethylenetriamine (dien), occurring as cationic dienH₂²⁺ at pH 7.5 (Watt an Cude, 1968), forms only labile ionic bonds with nucleic acids and does not affect the electrophoretic mobility of the oligonucleotides (FIG. 21, lanes 1 and 12). However, when the reaction mixtures contained 3 mM dien, we observed a modification of the oligonucleotides, with about 50% yield of a species moving more slowly in the gel (FIG. 21, lanes 3 and 14). Such gel-mobility shift indicates a covalent attachment of the positively charged group(s) to the oligonucleotides. The dienh₂²⁺ apparently counteracts the electrostatic repulsion between [PtCl₄]²⁻ and oligonucleotide polyanion, bringing them together through ionic linkages and providing an efficient concentration of reactive platinum species in vicinity of oligonucleotide resulted in platination of the oligonucleotide (FIG. 16). The platination of TT [d(TTCCTCTTTGGGGTGT) and TST [d(TTCCTCTTₛTGGGGTGT) is in line with the known high reactivity of Gₙ clusters and the POS moiety towards platinum(II) reagents (Elmroth and Lippard, 1994; Gonnet et al., 1996).

Plausible structure of platinum groups tethered to oligonucleotides. There are two possible pathways by which a bimolecular reaction proceeds first in such preformed [chloroplatinate-dien-oligonucleotide] complexes with high local concentrations of the reactants. The first pathway is a reaction between [PtCl₄]²⁻ and dienH₂²⁺ yielding [(dien)PtCl]⁺, although this reaction proceeds very slow even in highly concentrated solutions (Watt an Cude, 1968; Mahal and Van Eldick, 1987). The second possible pathway is direct reaction between [PtCl₄]²⁻ (or products of its aquotation) and nucleophilic atoms available in the oligonucleotide, followed by binding between the tethered platinum group and dienH₂²⁺ associated with the negatively charged nucleic acid surface. It is known that the products of the initial binding of [PtCl₄]²⁻ to polynucleotides are heterogeneous, unstable and very reactive (Chu and Orgel, 1990a; Kasianenko et al., 1995). Moreover, both POS sulfur and guanine N7 can additionally activate the tethered platinum groups due to their strong trans-influence (Howe-Grant and Lippard, 1980). To distinguish these two pathways, we studied an effect of increasing concentrations of Cl⁻ on the platination of TT and TST oligonucleotides (FIG. 21, lanes 8-11 and 19-22). The reaction between [(dien)PtCl]⁺ and the POS sulfur is known to be nearly independent of the Cl⁻ concentration whereas the reaction with guanine N7 can be completely inhibited at high concentrations of NaCl (Reedijk, 1991; Slavin et al. 1994). Indeed, we observed inhibition of the platination of both TT and TST oligonucleotides at 100 mM concentration NaCl (FIG. 21, lanes 11 and 22). Moreover, a pre-incubation of the [chloroplatinate-dien] mixtures at different conditions before mixing with the oligonucleotides did not accelerate the platination of oligonucleotides (data not shown). These results suggest the second pathway.

Interestingly, the presence of 1 mM NaCl in the reaction mixture does not reduce the platination yield (FIG. 21, lanes 9 and 20) but, in the case of TT oligonucleotide, makes the product band sharper than without NaCl (FIG. 21, lanes 9 and 3). Since the mobilities of the products formed by platination of both TT and TST oligonucleotides were identical even under the high resolution 20% gel-electrophoresis, we assume that the structure of the tethered platinum groups is the same in both cases. Platination reactions carried out in the presence of ligands forming very strong bonds with platinum, such as anionic I⁻ (FIG. 21, lanes 4-7 and 15-18), and neutral thiourea, did not change the gel-mobility of the product. Therefore, these ligands cannot compete with dien (a very strong chelating agent) for the binding of the tethered platinum groups. However, they can inhibit the initial reaction of platinum binding to the oligonucleotides at high concentrations (FIG. 21, lanes 7 and 18). We suggest that the platinum group attached is [(dien)Pt]²⁺, which is known to form chemically inert and thermodynamically stable adducts through both sulfur of POS or N7-position of guanine.

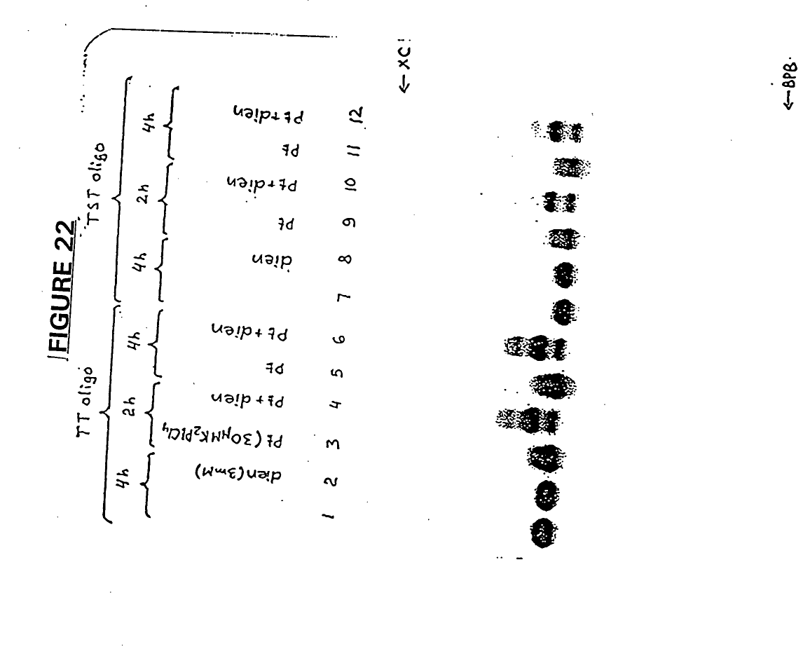

What metal binding center is more reactive: POS or Gₙ clusters? The longer reaction time at 45° C. provides a higher yield of platination of both TT and TST oligonucleotides after 2 h incubation (FIG. 22, lanes 4 and 10), than that after 1 h (used for the experiments presented on FIG. 21), but showed no further increase after 4 h incubation (FIG. 22, lanes 6 and 12). It also revealed that the platination of TST (FIG. 22, lanes 10 and 12) proceeded more specifically than of TT (FIG. 22, lanes 4 and 6), which showed formation of additional product bands. Interestingly, platination of a similar model system, S-guanosyl-L-homocysteine (GSH), showed a kinetic preference for the cysteine sulfur modification over the guanine N7 (Bloemink and Reedijk, 1996). Elmroth and Lippard (1994) showed that the rate of platination of the guanine N7 at the GG site in d(TTTTTTTTGGTTTTTTTT) by cis-[Pt(NH₃)(NH₂C₆H₁₁)Cl(H₂O)]⁺ is only 3-fold less than the POS sulfur in d(TTTTTTT_{S}TTTTTTT). In Gₙ clusters (n>3), the N7 of the central guanine residues are more nucleophilic than the flanking residues (Yohannes et al., 1993), and apparently more reactive than the N7 in GG. In contrast to homopyrimidine phosphorothioate oligonucleotides where selective platination of POS residues by K₂[PtCl₄] is known to occur (Chu and Orgel, 1989, 1990, 1992), no published data regarding platination of G-rich phosphorothioate oligonucleotides is currently available. We believe that the platination of the TST and STT [d(T_{S}T_{S}C_{S}C_{S}T_{S}C_{S}T_{S}T_{S}T_{S}G_{S}G_{S}G_{S}G_{S}T_{S}G_{S}T)] oligonucleotides containing both phosphorothioate group(s) and GGGG clusters may result in a mixture of Pt—S and Pt-N7(Gua) adducts.

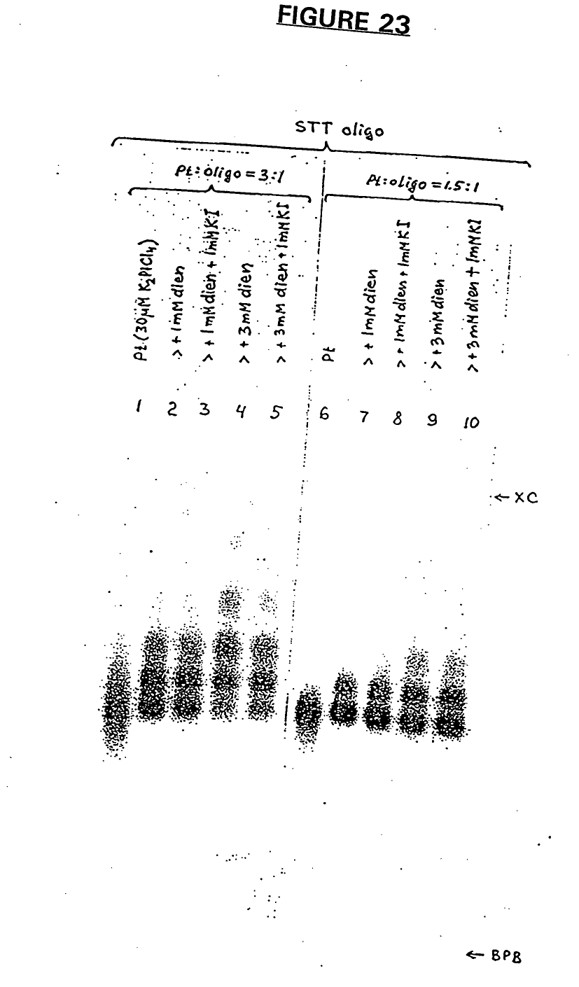

However, we obtained an evidence that the POS residues are more reactive than the GGGG cluster. We showed that the platination of all-phosphorothioate STT under the same conditions as for TT and TST oligonucleotides {30 μM K₂[PtCl₄], 10 μM oligonucleotide (molar ratio Pt:oligonucleotide=3:1) and 3 mM dien}forms a three-band ladder (FIG. 23, lanes 4 and 5) while TT and TST oligonucleotides form just one major band. Moreover, in the presence of 20 μM STT (molar ratio Pt:oligonucleotide=1.5:1), we found formation of only two distinct product bands (FIG. 23, lanes 4 and 5). We assume the number of the homogeneous bands corresponds to the number of tethered platinum groups.

The additional positive charge of the platinum groups also allows an easy separation of platinated oligonucleotides from reaction mixtures by preparative electrophoresis. As an alternative isolation method we might consider ion-exchange column chromatography. We also suggest using of metallo-affinity chromatography on mercurated columns as a one-step method of purification of platinated phosphorothioate oligonucleotides.

Optimum conditions for platination of phosphorothioate and G-rich oligonucleotides:
10 μM oligonucleotide
30 μM K₂PtCl₄*
3 mM dien
10 mM Tris-OAc, pH 7.5
1 mM Na₃EDTA
1 mM NaCl
Incubation for 2 h at 45° C.

The contents of the optimal platination mixtures (10 μl) is:
1 μl of ³²P-labeled oligonucleotide in 1×TE
1 μl of 100 μM non-radioactive oligonucleotide in 1×TE
2 μl of 150 μM K₂PtCl₄*
2 μl of 15 mM dien in 50×TAE
2 μl of 5 mM NaCl
2 μl of H₂O

(* fresh solution in H₂O pre-incubated for 1 h at 45° C. before adding to the reaction mixture).

In looking for the optimal conditions, we found prominent effects of a variety of components of reaction mixtures on the platination yield and number of products formed.

Dien. The higher the dien concentration, the better the yield of the platination. Compare, for example, the product yields in the presence of 1 mM dien and 3 mM dien (FIG. 23, lanes 2-5). However, at concentrations above 3 mM the reaction yield declines presumably because of the precipitation of oligonucleotides.

Pt and oligonucleotide concentrations. For preparative bimolecular (second-order) reactions the concentrations of both components are important. In our case the amount of radioactive platinum (and therefore its concentration) is a limiting factor. We can not resolve this limitation by using an excess of oligonucleotide components (making of pseudo-first order reaction) because of possible oligonucleotide precipitation and problems with isolation of platinated oligonucleotides. We determined the allowable concentration range for K₂PtCl₄ to be 3 to 100 μM in 10 μl reaction mixtures (0.03 to 1 nmoles platinum); below that level we did not detect any platination of oligonucleotides for reasonable reaction time, whereas above that level we observed fast non-specific modification and precipitation of oligonucleotides. The optimal [Pt:oligonucleotide] molar ratio in reaction mixtures was determined to be useful in the range Of 1.5 to 6 (FIG. 24, lanes 5-7).

K₂PtCl₄ pre-treatment. It was shown that the transition of [PtCl₄]²⁻ to its aquo-complexes [PtCl₃(H₂O)]⁻ and [PtCl₂(H₂O)₂]⁰ affected its binding with DNA (Kasianenko et al., 1995). Aquotation starts immediately in freshly prepared K₂PtCl₄ solutions, and we found that this process can affect on the reproducibility of our experiments. Therefore, we recommend pre-incubating the fresh stock solutions of K₂PtCl₄ (10 mM) for 1 h at 45° C. to complete this process before using for the oligonucleotide modification.

The foregoing description details specific methods which can be employed to practice the present invention. Having detailed such specific methods, those skilled in the art will well enough know how to devise alternative reliable methods at arriving at the same information in using the fruits of the present invention. Thus, however, detailed the foregoing may appear in text, it should not be construed as limiting the overall scope thereof; rather, the ambit of the present invention is to be determined only by the lawful construction of the appended claims. All documents cited herein are expressly incorporated by reference.

### BIBLIOGRAPHY

Alumni-Fabbroni, M., Manfioletti, G., Manzini, G., and Xodo, L. E. (1994) Inhibition of T7 RNA polymerase transcription by phosphate and phosphorothioate triplex-forming oligonucleotides targeted to a RAY site downstream from the promoter. Eur. J. Biochem. 226, 831-839.
Anand, D. and Wolf, W. (1992) A new, semi-automated system for the microscale synthesis of [^{195m}Pt]cis platin suitable for clinical studies. Appl. Radiat. Isot. (Int. J. Radiat. Appl. Instrum., Part A) 43, 809-814.
Azure, M. T., Sastry, K. S. R., Archer, R. D., Howell, R. W., and Rao, D. V. (1992) Microscale synthesis of carboplatinum labeled with the Auger emitter platinum-193m: radiotoxicity versus chemotoxicity of antitumor drug in mammalian cells. in Biophysical Aspects of Auger Processes (AAPM Symp. Ser. No. 8), R. W. Howell et al., eds., pp. 336-351.
Beal, P. A., and Dervan, P. B. (1992) Recognition of double helical DNA by alternate strand triple helix formation. J. Am. Chem. Soc. 114, 4976-4982.
Brabec, V., Boudny, V., and Barcalov‡, Z. (1994) Monofunctional adducts of platinum(II) produce in DNA a sequence-dependent local denaturation. Biochemistry 33, 1316-1322.
Brosalina, E. B., Pascolo, E., and Toulm, J.-J. (1993) The binding of an antisense oligonucleotide to a hairpin structure via triplex formation inhibits chemical and biological reactions. Nucleic Acids Res. 21, 5616-5622.
Bruhn, S. L., Toney, J. H., and Lippard, S. J. (1990) Biological processing of DNA by platinum compounds. in Progress in Inorganic Chemistry: Bioinorganic Chemistry, Vol. 38, S. J. Lippard, ed., Wiley, pp. 477-516. Cheng, Y.-K., and Pettitt, B. M. (1992) Stabilities of double- and triple-strand helical nucleic acids. Prog. Biophys. Molec. Biol. 58, 225-257.
Chu, B. C., and Orgel, L. E. (1994) Postsynthesis functionalization of oligonucleotides. Methods Mol. Biol. 26, 145-165.
Chu, B. C. F., and Orgel, L. E. (1989) Inhibition of DNA synthesis by cross-linking the template to platinum-thiol of complementary oligodeoxynucleotides. Nucleic Acids Res. 17, 47834798.
Chu, B. C. F., and Orgel, L. E. (1990a) Optimization of the efficiency of cross-linking Pt(II) complexes to complementary oligodeoxynucleotides. Nucleic Acids Res. 18, 5163-5171.
Chu, B. C. F., and Orgel, L. E. (1990b) A simple procedure for cross-linking complementary oligonucleotides. DNA and Cell Biology 9, 71-76.
Gruff, E. S., and Orgel, L. E. (1991) An efficient, sequence-specific method for crosslinking complementary oligonucleotides using binuclear platinum complexes. Nucleic Acids Res. 19, 6849-6854.
Chu, B. C. F., and Orgel, L. E. (1992) Crosslinking transcription factors to their recognition sequences with Pt(II) complexes. Nucleic Acids Res. 20, 2497-2502.
Conway, N. E., and McLaughlin, L. W. (1991) The covalent attachment of multiple fluorophores to DNA containing phosphorothioate diesters results in highly sensitive detection of single-stranded DNA. Bioconjug. Chem. 2, 452457.
Conway, N. E., Fidanza, J., and McLaughlin, L. W. (1989) The introduction of reporter groups at multiple and/or specific sites in DNA containing phosphorothioate diesters. Nucleic Acids Symp. Ser. 21, 43-44.
Eckstein, F. (1983) Phosphorothioate analogues of nucleotides D tools for the investigation of biochemical processes. Angew. Chem. Int. Ed. Engl. 22, 423-439.
Eckstein, F. (1985) Nucleoside phosphorothioates. Ann. Rev. Biochem. 54, 367-402.
Ellouze, C., Piot, F., and Takashi, M. (1997) Use of fluorescein-labeled oligonucleotide for analysis of formation and dissociation kinetics of T:A:T triple-stranded DNA: effect of divalent cations. J. Biochem. 121, 521-526.
Elmroth, S. K. C., and Lippard, S. J. (1994) Platinum binding to d(GpG) target sequence and phosphorothioate linkages in DNA occurs more rapidly with increasing oligonucleotide length. J. Am. Chem. Soc. 116, 3633-3634.
Francois, J. C., and Helene, C. (1995) Recognition and cleavage of single-stranded DNA containing hairpin structures by oligonucleotides forming both Watson-Crick and Hoogsteen hydrogen bonds. Biochemistry 34, 65-72.
Francois, J. C., Saison-Behmoaras, T., Barbier, C., Chassignol, M., Thuong, N. T., Hel n, C. (1989) Sequence-specific recognition and cleavage of duplex DNA via triple-helix formation by oligonucleotides covalently linked to a phenanthroline-copper chelate. Proc. Natl. Acad. Sci. USA 86, 9702-9706.
Frey, P. A., and Sammons, R. D. (1985) Bond order and charge localization in nucleoside phosphorothioates. Science 228, 541-545.
Fidanza, J. A., Ozaki, H., McLaughlin, L. W. (1994) Functionalization of oligonucleotides by the incorporation of thio-specific reporter groups. Methods Mol Biol. 26, 121-143.
Gonnet, F., Reeder, F., Kozelka, J., and Chottard, J.-C. (1996) Kinetic analysis of the reactions between GG-containing oligonucleotides and platinum complexes. 1. Reactions of single-stranded oligonucleotides with cis-[Pt(NH₃)₂(H₂O)₂]²⁺ and [Pt(NH₃)₃(H₂O)]²⁺. Inorg. Chem. 35, 1653-1658.
Gerstner, J. A., Pedroso, P., Morris, J., and Bergot, B. J. (1995) Gram-scale purification of phosphorothioate oligonucleotides using ion-exchange displacement chromatography. Nucleic Acids Res. 23, 2292-2299.
Gueron, M., and Weibuch, G. (1981) Polyelectrolyte theory of charged-ligand binding to nucleic acids. Biochimie 63, 821-825.
Haner, R., and Hall, J. (1997) The sequence-specific cleavage of RNA by artificial chemical ribonucleases. Antisense Nucleic Acid Drug Dev. 7, 423-430.
Hashem, G. M., Pham, L., Vaughan, M. R., and Gray D M (1998) Hybrid oligomer duplexes formed with phosphorothioate DNAs: CD spectra and melting temperatures of S-DNAáRNA hybrids are sequence-dependent but consistent with similar heteronomous conformations. Biochemistry 37, 61-72.
Helene C. (1993) Sequence-selective recognition and cleavage of double-helical DNA. Curr. Opin. Biotechnol. 4, 29-36.
Hodges, R. R., Conway, N. E., and McLaughlin, L. W. (1989) “Post-assay” covalent labeling of phosphorothioate-containing nucleic acids with multiple fluorescent markers. Biochemistry 28, 261-267.
Hoeschele, J. D., Butler, T. A., and Roberts, J. A. (1980) Correlation of physico-chemical and biological properties with in vivo biodistribution data for platinum-195m-labeled chloroaminoplatinum(II) complexes. in Inorganic Chemistry in Biology and Medicine. (ACS Symp. Ser., Vol. 140), A. E., Martell, ed., ACS, Washington, D.C., pp. 181-208.
Howe-Grant, M. E., and Lippard, S. J. (1980) Aqueous platinum(II) chemistry: binding to biological molecules. in Metal ions in Biological Systems, Vol. 11, H. Sigel, ed., Marcel Dekker, New York—Basel, pp. 63-125.
Ide, G. J. (1981) Nucleoside 5′-[gamma-S]triphosphates will initiate transcription in isolated yeast nuclei. Biochemistry 20, 2633-2638.
Igloi, G. L. (1988) Interaction of tRNAs and of phosphorothioate-substituted nucleic acids with an organomercurical. Probing the chemical environment of thiolated residues by affinity electrophoresis. Biochemistry 27, 3842-3849.
Jaroszewski, J. W., Syi, J. L., Maizel, I., and Cohen, J. S. (1992) Towards rational design of antisense DNA: molecular modeling of phosphorothioate DNA analogues. Anticancer Drug Des. 7, 253-262.
Jayasena, S. D., and Johnston, B. H. (1992) Oligonucleotide-directed triple helix formation at adjacent oligopurine and oligopyrimidine DNA tracts by alternate strand recognition. Nucleic Acids Res. 20, 5279-5288.
Jones, A. S., Walker, R. T., and Youngs, V. (1973) Preparation of a stable mercury derivative of tyrosine transfer RNA. Biochim. Biophys. Acta 299, 293-299.
Joseph, J., Kandala, J. C., Veerapanane, D., Weber, K. T., and Guntaka, R. V. (1997) Antiparallel polypurine phosphorothioate oligonucleotides form stable triplexes with the rat alpha1(I) collagen gene promoter and inhibit transcription in cultured rat fibroplasts. Nucleic Acids Res. 25, 2182-2188.
Kandimalla, E. R., Agrawal, S., Venkataraman, G., and Sasisekharan, V. (1995) Single strand targeted triplex formation: parallel-stranded DNA hairpin duplexes for targeting pyrimidine strands. J. Am. Chem. Soc. 117, 6416-6417.
Kanehara, H., Nishi, M., and Makino, K. (1995) Solution structure of the duplexes between phosphorothioate DNA and target DNA/RNA. Nucleic Acids Symp. Ser. 34, 53-54.
Kasianenko, N. A., Karymov, M. A., DOiachenko, S. A., Smorygo, N. A., and Frisman, E. V. (1995) Interaction of DNA molecules with divalent platinum coordination complexes. II. Effect of the nature and location of ligands in the first platinum coordination sphere. Mol. Biol. (Mosk.) 29, 585-596.
Kazakov, S. A. (1996) Nucleic Acid Binding and Catalysis by Metal Ions. in Bioorganic Chemistry. Part I: Nucleic Acids, S. M. Hecht, ed., Oxford University Press, NY, pp. 244-287 & 467-476.
Kessler, D. J., Pettitt, B. M., Cheng, Y. K., Smith, S. R., Jayaraman, K., Vu, H. M., Hogan, M. E. (1993) Triple helix formation at distant sites: hybrid oligonucleotides containing a polymeric linker. Nucleic Acids Res. 21, 4810-4815.
Kibler-Herzog, L., Zon, G., Uznanski, B., Whittier, G., and Wilson, W. D. (1991) Duplex stability of phosphorothioate, methylphosphonate, and RNA analogs of two DNA 14-mers. Nucleic Acids Res. 19, 2979-2986
Kim, S. G., Tsukahara, S., Yokoyama, S., and Takaku, H. (1992) The influence of oligodeoxyribonucleotide phosphorothioate pyrimidine strands on triplex formation. FEBS Lett. 314, 29-32.
Latimer, L. J. P., Hampel, K., and Lee, S. (1989) Synthetic repeating sequence DNAs containing phosphorothioates: nuclease sensitivity and triplex formation. Nucleic Acids Res. 17, 1549-1560.
Lacoste, J., Fran ois, J.-C., and Hel n, C. (1997) Triplex helix formation with purine-rich phosphorothioate-containing oligonucleotides covalently linked to an acridine derivatives. Nucleic Acids Res. 25, 1991-1998.
Lepre, C. A., and Lippard, S. J. (1990) in Nucleic Acids and Molecular Biology, Vol. 4, F. Eckstein and D. M. J. Lilley, Springer-Verlag, Berlin—Heidelberg, pp. 9-37.
Liang, C., and Allen, L. C. (1987) Sulfur does not form double bonds in phosphorothioate anions. J. Am. Chem. Soc. 109, 6449-6453.
Lippard, S. J. (1978) Platinum complexes; probes of polynucleotide structure and antitumor drugs. Acc. Chem. Res. 11, 211-217.
Mahal, G., and Van Eldik, R. (1987) Preparation, characterization and reactivity of a series of diethylenetriamine (dien) and substituted complexes of platinum(II). Inorg. Chim. Acta 127, 203-208.
Malkov, V. A., Voloshin, O. N., Soyfer, V. N., and Frank-Kamenetskii, M. D. (1993) cation and sequence effects on stability of intermolecular pyrimidine-purine-purine triplex. Nucleic Acids Res. 21, 585-591.
Melvin, W. T., and Keir, H. M. (1979) Interaction of 6-thiopurines and thiol-containing RNA with a cellulose mercurial. Anal. Biochem. 92, 324-330.
Moser, H. E., Dervan, P. B. (1987) Sequence-specific cleavage of double helical DNA by triple helix formation. Science 238, 645-650.
Moses, A. C., and Schepartz, A. (1996) Triplex tethered oligonucleotide probes. J. Am. Chem. Soc. 118, 10896-10897.
Musso, M., and Van Dyke, M. W. (1995) Polyamine effects on purine-purine-pyrimidine triple helix formation by phosphodiester and phosphorothioate oligodeoxyribonucleotides. Nucleic Acids Res. 23, 2320-2327.
O'Donnel, M. J., and McLaughlin, L. W. (1996) Reporter groups for the analysis of nucleic acid structure. in Bioorganic Chemistry. Part I: Nucleic Acids, S. M. Hecht, ed., Oxford University Press, NY, pp. 216-243.
Ozaki, H., and McLaughlin, L. W. (1992) The estimation of distances between specific backbone-labeled sites in DNA using fluorescence resonance energy transfer. Nucleic Acids Res. 20, 5205-5214.
Olivas, W. M., and Maher, III, L. J. (1995a) Competitive triplex/quadruplex equilibria involving guanine-rich oligonucleotides. Biochemistry 34, 278-284.
Olivas, W. M., and Maher, III, L. J. (1995b) Overcoming potassium-mediated triplex inhibition. Nucleic Acids Res. 23, 1936-1941.
Padmapriya, A. A., Tang, J., and Agrawal, S. (1994) Large-scale synthesis, purification and analysis of oligodeoxynucleotide phosphorothioates. Antisense Res. Dev. 4, 185-199.
Pal, B. C., Schugart, L. R., Isham, K. R., and Stulberg, M. P. (1972) Modification of 4-thiouridine and phenylalanine transfer RNA with parachloromercuribenzoate. Archives Biochem. Biophys. 150, 86-90.
Pallan, P. S., and Ganesh, K. N. (1996) DNA triple helix stabilization by bisguanidinyl analogues of biogenic polyamines. Biochem. Biophys. Res. Commun. 222, 416-420.
Plum, G. E., and Pilch, D. S. (1995) Nucleic acid hybridization: triplexes stability and energetic. Annu. Rev. Biophys. Biomol. Struct. 24, 319-350.
Reeder, F., Kozelka, J., and Chottard, J.-C. (1996) Triamminoplatinum(II) coordinated to a guanine does not prevent platination of an ajacent guanine in single-stranded oligonucleotides. Inorg. Chem. 35, 1413-1415.
Reedijk, J. (1991) New insights about the interaction of cis-platinum with intracellular components. in Platinum and Other Metal Coordination Compounds in Cancer Chemotherapy, S. B. Howell, ed., Plenum Press, New York, pp. 13-23.
Richardson, C. C. (1981) Bacteriophage T4 polynucleotide kinase. in The Enzymes 14, 299-314.
Scheit, K.-H., and Faerber, P. (1973) The interaction of 2-thiopyrimidine bases with hydroxymercurybenzene sulfonate. Eur. J. Biochem. 33, 545-550.
Sherman, S. E., and Lippard, S. J. (1987) Structural aspects of platinum anticancer drug interactions with DNA. Chem. Rev. 87, 1153-1181.
Shimizu, M., Inoue, H., Ohtsuka, E. (1994) Detailed study of sequence-specific DNA cleavage of triplex-forming oligonucleotides linked to 1,10-phenanthroline. Biochemistry 33, 606-613.
Slavin, L. L., Cox, E. H., and Bose, R. N. (1994) Platinum(II)-adenosine phosphorothioate complexes: kinetics of formation and phosphorus-31 NMR characterization studies. Bioconjug. Chem. 5, 316-320.
Smith, M. M., and Huang, R. C. (1976) Transcription in vitro of immunoglobulin kappa light chain genes in isolated mouse myeloma nuclei and chromatin. Proc. Natl. Acad. Sci. USA 73, 775-779.
Smith, M. M., Reeve, A. E., and Huang, R. C. (1978a) Analysis of RNA initiated in isolated mouse myeloma nuclei using purine nucleoside 5′[gamma-S]triphosphates as affinity probes. Cell 15, 615-626.
Smith, M. M., Reeve, A. E., and Huang, R. C. (1978b) Transcription of bacteriophage lambda DNA in vitro using purine nucleoside 5′-[gamma-S]triphosphates as affinity probes for RNA chain initiation. Biochemistry 17, 493-500.
Stepanek, J., Larrson, B., and Weinreich, R. (1996) Auger-electron spectra of radionuclides for therapy and diagnostics. Acta Oncologica 35, 863-868.
Strothkamp, K. G., and Lippard, S. J. (1976) Platinum binds selectively to phosphorothioate groups in mono- and polynucleotides: a general method for heavy metal staining of specific nucleotides. Proc. Natl. Acad. Sci. USA 73, 2536-2540.
Strothkamp, K. G., Lehmann, J., and Lippard, S. J. (1978) Tetrakis(acetooxymercuri)methane: a polymetallic reagent for labeling sulfur in nucleic acids. Proc. Natl. Acad. Sci. USA 75, 1181-1184.
Sun, I. Y., and Allfrey, V. G. (1982) Labeling and selective recovery of newly synthesized viral DNA from simian virus 40-infected cells incubated with inorganic thiophosphate. Proc. Natl. Acad. Sci. USA 79, 4589-4593.
Sun, I. Y., Johnson, E. M., and Allfrey, V. G. (1982) Initiation of transcription of ribosomal deoxyribonucleic acid sequences in isolated nuclei of Physarum polycephalum: studies using nucleoside 5′-[gamma-S]triphosphates and labeled precursors. Biochemistry 18, 4572-4580.
Svinarchuk, F., Cherny, D., Debin, A., Delain, E., and Malvy, C. (1996) A new approach to overcome potassium-mediated inhibition of triplex formation. Nucleic Acids Res. 24, 3858-3865.
Szalda, D. J., Eckstein, F., Sternbach, H., and Lippard, S. J. (1979) Specific heavy metal labeling of the 3′-terminus of phosphorothioate modified yeast tRNA^{Plc}. J. Inorg. Biochem. 11, 279-282.
Tavitian, B., Terrazzino, S., Kuhnast, B., Marzabal, S., Stettler, O., Dolle, F., Deverre, J. R., Jobert, A., Hinnen, F., Bendriem, B., Crouzel, C., Di Giamberardino, L. (1998) In vivo imaging of oligonucleotides with positron emission tomography. Nat. Med. 4, 467-471.
Thomas, T. and Thomas, T. J. (1993) Selectivity of polyamines in triplex DNA stabilization. Biochemistry 32, 14068-14074.
Thuong, N. T., and Hel n, C. (1993) Sequence-specific recognition and modification of double-helical DNA by oligonucleotides. Angew. Chem. Int. Ed. Engl. 32, 666-690.
Tsukahara, S., Kim, S. G., and Takaku, H. (1993) Inhibition of restriction endonuclease cleavage site via triple helix formation by homopyrimidine phosphorothioate oligonucleotides. Biochem. Biophys. Res. Commun. 196, 990-996.
Tsukahara, S., Suzuki, J., Ushijima, K., Takai, K., and Takaku, H. (1996) Nonenzymatic sequence-specific cleavage of duplex DNA via triple-helix formation by homopyrimidine phosphorothioate oligonucleotides. Bioorg. Med. Chem. 4, 2219-2224.
Tsukahara, S., Suzuki, J., Hiratou, T., Takai, K., Koyanagi, Y., Yamomoto, N., and Takaku, H. (1997) Inhibition of HIV-1 replication by triple-helix-forming phosphorothioate oligonucleotides targeted to the polypurine tract. Biochem. Biophys. Res. Commun. 233, 742-747.
Tung, C.-H., Breslauer, K. J., and Stein, S. (1993) Polyamine-linked oligonucleotides for DNA triple helix formation. Nucleic Acids Res. 21, 5489-5494.
Tung, C.-H., Breslauer, K. J., and Stein, S. (1996) Stabilization of DNA triple-helix formation by appended cationic peptides. Bioconjug. Chem. 7, 529-531.
Washington, L. D., and Stallcup, M. R. (1982) A comparison of nucleoside (beta-S)triphosphates and nucleoside (gamma-S)triphosphates as suitable substrates for measuring transcription initiation in preparations of cell nuclei. Nucleic Acids Res. 10, 8311-8322.
Watt, G. W., and Cude, W. A. (1968) Diethylenetriamine complexes of platinum(II) halides. Inorg. Chem. 7, 335-338.
Wherland, S., Deutsch, E., Eliason, J., and Sigler, P. B. (1973) Interactions between polynucleotides and platinum(II) complexes. Biochem. Biophys. Res. Commun. 54, 662-668.
Wilson, W. D., Tanious, F. A., Mizan, S., Yao, S, Kiselyov, A. S., Zon, G., and Strekowski, L. (1993) DNA triple-helix specific intercalators as antigene enhancers: unfused aromatic cations. Biochemistry 32, 10614-10621.
Xodo, L. E., Alumni-Fabbroni, M., Manzini, G., and Quadrifoglio, F. (1994) Pyrimidine phosphorothioate oligonucleotides form triple-stranded helices and promote transcription inhibition. Nucleic Acids Res. 22, 3322-3330.
Yohannes, P. G., Zon, G., Doetsch, P. W, and Marzilli, L. G. (1993) DNA hairpin formation in adducts with platinum anticancer drugs: gel-electrophoresis provides new information and a caveat. J. Am. Chem. Soc. 115, 5105-5110.
Zhang, Z. Y., Thompson, E. A., and Stallcup, M. R. (1984) Hormonal regulation of transcription of rDNA: use of nucleoside thiotriphosphates to measure initiation in isolated nuclei. Nucleic Acids Res. 12, 8115-8128.
Zon, G., and Geiser, T. G. (1991) Phosphorothioate oligonucleotides: chemistry, purification, analysis, scale-up and future directions. Anticancer Drug Des. 6, 539-568.
Zon, G., and Stec, W. J. (1991) Phosphorothioate oligonucleotides. in Oligonucleotides and Analogues. A Practical Approach, ed. Eckstein, F., IRL Press at Oxford University Press, Oxford—NY—Tokyo, pp. 87-108.

1. A nucleic acid molecule or analog thereof which specifically binds to a target nucleic acid through a sequence which is substantially complementary to a sequence of the target nucleic acid, said nucleic acid molecule or analog thereof being capable of topologically linking to at least a portion of said target nucleic acid through the interaction between the 5′ and 3′ ends of said nucleic acid molecule or analog thereof, said topological linkage effectively reducing the efficiency of transcription or translation of said target nucleic acid.

2. The nucleic acid molecule or analog thereof of claim 1 which comprises catalytic RNA.

3. The nucleic acid molecule or analog thereof of claim 2, wherein said catalytic RNA is a catalytic region derived from the hairpin ribozyme.

4. The nucleic acid molecule or analog thereof of claim 2, wherein said catalytic RNA is connected to said sequence which is substantially complementary to a sequence of the target nucleic acid through a bond or a linker.

5. The nucleic acid molecule or analog thereof of claim 2 which further comprises a triplex-forming region that interacts with the duplex formed between said nucleic acid molecule or analog thereof and said target molecule through the formation of Hoogsteen or reverse Hoogsteen bonds.

6. The nucleic acid molecule or analog thereof of claim 5, wherein the triplex-forming region forms one fewer helical turn than said duplex so as to ensure topological linkage of said nucleic acid molecule or analog thereof to said target nucleic acid.

7. The nucleic acid molecule or analog thereof of claim 1, wherein said interaction between the 5′ and 3′ ends of said nucleic acid molecule or analog thereof is by covalent bonding, Watson-Crick pairing, Hoogsteen bonding, reverse Hoogsteen bonding or other noncovalent interaction.

8. The nucleic acid molecule or analog thereof of claim 1, wherein said 5′ and said 3′ ends of said nucleic acid molecule or analog thereof do not interact until after said nucleic acid molecule or analog thereof binds to said target nucleic acid.

9. A nucleic acid molecule or analog thereof which specifically binds to a target nucleic acid through a sequence which is substantially complementary to a sequence of the target nucleic acid, wherein the 5′ and 3′ ends of said nucleic acid molecule or analog thereof interact to form a binding site for a locking molecule and wherein the binding of said locking molecule to said binding site causes said nucleic acid molecule or analog thereof to become topologically linked to said target nucleic acid, thereby effectively reducing the efficiency of transcription or translation of said target nucleic acid.

10. The nucleic acid molecule or analog thereof of claim 9 which is RNA or DNA.

11. The nucleic acid molecule or analog thereof of claim 9 which interacts with said target nucleic acid by Watson-Crick base pairing, Hoogsteen base pairing or reverse Hoogsteen base pairing.

12. The nucleic acid molecule or analog thereof of claim 9, wherein said 5′ and 3′ sequences which interact to form said binding site are identified through combinatorial library screening.

13. The nucleic acid or analog thereof of claim 9, wherein said locking molecule is selected from the group consisting of a protein, a metal ion, a metal complex, a drug, an organic molecule and an inorganic molecule.

14. The nucleic acid molecule or analog thereof of claim 13, wherein said protein is c-myc, n-myc or 1-myc.

15. The nucleic acid molecule or analog thereof of claim 13 wherein said locking molecule is zinc-, copper-, cobalt- or a platinum-containing molecule.

16. The nucleic acid molecule or analog thereof of claim 9 which comprises at least one phosphorothioate internucleotide linkage.

17. The nucleic acid molecule or analog thereof of claim 9 which comprises a competing internal structure which is competed out by binding of said locking molecule.

18. A method for inhibiting the transcription or translation of a target nucleic acid molecule, said method comprising:
contacting said target nucleic acid molecule with a nucleic acid molecule or analog thereof according to claim 1, wherein said nucleic acid molecule or analog thereof becomes topologically linked to said target nucleic acid molecule, thereby inhibiting transcription or translation therefrom.

19. A method of covalently bonding a platinum molecule to an oligonucleotide, said method comprising:
combining said oligonucleotide with (1) a platinum donor molecule and (2) a positively charged polyamine molecule under conditions that allow covalent bonding of said platinum molecule to said oligonucleotide,
wherein said platinum molecule becomes covalently bound to said oligonucleotide.

20. The method according to claim 19, wherein said platinum donor molecule and said positively charged polyamine molecule are complexed prior to combining with said oligonucleotide.

21. The method according to claim 19, wherein said platinum molecule becomes covalently bound to a sulfur molecule or an N7 of a guanine residue of said oligonucleotide.

22. An oligonucleotide obtained by the method according to claim 19.

23. (canceled)
